(19) United States
(12) Patent Application Publication
(10) Pub. No.: US 2015/0147274 A1
(43) Pub. Date: May 28, 2015

(54) ANTIBODIES AGAINST HGF - RECEPTOR AND USES

(71) Applicant: CANCER RESEARCH TECHNOLOGY LIMITED, London (GB)

(72) Inventors: Danielle Marie Di Cara, Cambridge (GB); John McCafferty, Cambridgeshire (GB); Ermanno Gherardi, Pavia (IT); Anthony Richard Pope, Cambridge (GB)

(21) Appl. No.: 14/362,221

(22) PCT Filed: Nov. 30, 2012
(86) PCT No.: PCT/GB2012/052980

§ 371 Date: Jun. 2, 2014

Related U.S. Application Data
(60) Provisional application No. 61/566,268, filed on Dec. 2, 2011.

(51) Int. Cl. C07K 16/28; C07K 16/40; A61K 39/395; A61K 47/10; A61K 49/00

(52) U.S. Cl. CPC C07K 16/2863; A61K 47/10; A61K 49/0004; A61K 39/3955; C07K 16/40; C07K 2317/565; C07K 2317/51; C07K 2317/515; C07K 2317/92; C07K 2317/56; C07K 2317/55; C07K 2317/24; C07K 2317/569; C07K 2317/76
USPC 424/9.1; 530/389.1; 530/387.9; 530/388.1; 530/387.3; 530/391.1; 536/23.53; 530/391.3; 530/391.7; 424/130.1; 435/320.1; 435/252.33; 435/334; 435/254.2; 435/419; 435/183

(57) ABSTRACT

Antibodies which are antagonists of the human HGF receptor (MET), wherein the antibodies specifically bind to amino acid residues 568-741 of human MET (SEQ ID No: 1) with high affinity.

The present invention generally relates to antibodies that bind to the hepatocyte growth factor receptor c-Met, and to their use, for example, in treating conditions such as cancer.

The receptor c-Met (also referred to herein as “MET”), a member of the tyrosine kinase superfamily, is the receptor for Hepatocyte Growth Factor/Scatter Factor (HGF/SF; also referred to herein as “HGF”). Binding of HGF to MET leads to receptor dimerisation or multimerisation, phosphorylation of multiple tyrosine residues in the intracellular region, catalytic activation, and downstream signalling. MET is also activated via ligand-independent mechanisms, including receptor over-expression, amplification, and mutation. MET activation enhances cellular proliferation, migration, morphogenesis, survival (including protection from apoptosis), and protease synthesis, characteristics that are associated with invasive cell phenotype and poor clinical outcomes and drug resistance in cancer patients. The MET signalling pathway is one of the most frequently dysregulated pathways in human cancers, and occurs in virtually all types of solid tumours.

Metastasis is a complex process that consists of multiple stages: dissociation of tumour cells from the primary tumour and their migration into adjacent tissues (invasion), their entrance into lymphatic and/or blood vessels (intravasation), their exit from the bloodstream (extravasation) and the final growth of secondary tumours (colonisation). The tyrosine kinase MET, and its ligand HOF, are critically involved in the early stages of migration of tumour cells, the process that initiates metastasis. HGF is secreted by cells in the tumour stroma and causes cancer cells expressing MET to dissociate from neighbours, acquire a highly motile phenotype and initiate invasion.

There is compelling evidence that this sequence of events is caused by HGF and MET (Birchmeier et al, 2003). First, HGF causes epithelial cells to lose contact with their neighbours and become highly motile inducing concurrent expression of an array of proteinases (plasminogen, u-PA, etc) required for tumour invasion (Jeffers et al, 1996). Second, overexpression of HGF or mutant and active forms of MET in transgenic mice causes multiple metastatic tumours of epithelial and mesenchymal origin (Takayama et al, 1997; Graveel et al, 2004). Third, studies in cancer patients have shown that metastasis selects for cells over-expressing MET or expressing a mutated and active form (Di Renzo et al, 2000). Fourth, MET mutations in cancer patients cause invasive carcinomas of the kidney (Schmidt et al, 1997), liver (Lorenzato et al, 2002) and lung (Kong-Beltran et al, 2006). Finally, MET amplification causes signalling via other receptor kinases, such as ERBB3 leading to drug resistance (Burgess et al, 2006). In summary, the evidence for the role of HGF and MET in cancer invasion is extensive (cell biological, genetic, clinical and pathological) and consistent. Thus, the key outcome of MET signalling is the migration of cancer cells at a distance from the primary tumour reaching the local lymphatic or blood capillaries where they initiate the later stages of metastasis.

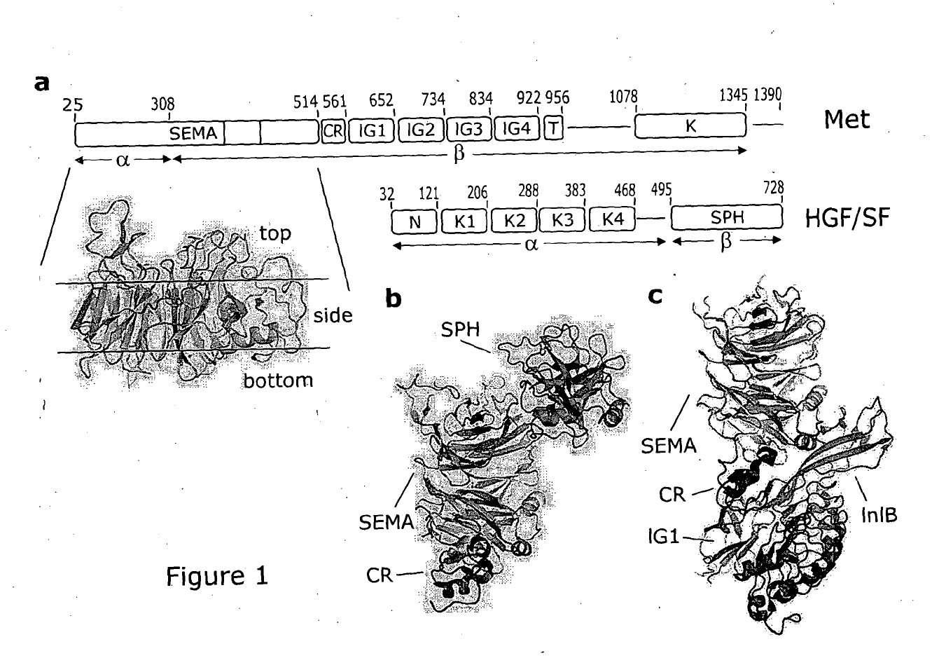

As shown in Box 1 of Trusolino et al (2010), and in FIG. 1 herein, the MET receptor has an ectodomain consisting of two chains produced by furin cleavage of a single chain precursor: an N-terminal a chain consisting of amino acid residues 25-307 and a C-terminal β chain consisting of amino acid residues 308-932. The β chain also encompasses the single transmembrane domain and the cytoplasmic, kinase domain. The N-terminal, 7-bladed β-propeller domain (also known as the Sema domain) was previously shown to be sufficient for binding HGF (Gherardi et al, 2003). The C-terminal region of the MET ectodomain consists of 4 immunoglobulin-like (IPT) domains, spanning about residues 562-922, in a flexible stalk structure. A small cysteine-rich domain separates the β-propeller domain and the stalk structure (Gherardi et al, 2003). This domain structure has been confirmed by small angle X-ray scattering, cryo-EM (Gherardi et al, 2006) and two crystal structures (Niemann et al, 2007; Stamos et al, 2004). The entire disclosure of Trusolino et al relating to MET and HGF structure, function and signalling is incorporated herein by reference.

MET can be targeted by a number of different approaches including the use of small molecule kinase inhibitors (e.g., ARQ197; Schiller ASCO 2010. J Clin Oncol 28: 18s (suppl; abstr LBA7502)).

Antagonistic antibodies which block MET signalling also have great potential to be effective agents in the treatment of cancer and much research has been devoted to obtaining antagonistic Met antibodies. Since the β-propeller/Sema domain is known as an important site of ligand binding, antibodies binding at this site could potentially interfere with ligand binding. In contrast it would be unexpected to find inhibition of signalling from antibodies targeted to the stalk region.

Several anti-MET antibodies with antagonistic activity are now available. For example, METMab (also known as onartuzumab and 5D5) is a single-arm antibody that binds the Sema domain of MET (Kong-Beltran et al, 2004) and displays antagonistic activity in a monovalent format (Jin et al, 2008). METMab/5D5 was reported to have an affinity for MET of 4 nM (Schwall et al, 2004, Proc. Amer. Assoc. Cancer Res. vol. 45, Abstr #1424), and appears to act by competing for the binding of HGF to MET. METMab/5D5, in combination with the EGFR inhibitor erlotinib increased progression-free survival in patients with non small-cell lung cancer with high levels of MET expression (Spigel et al, 2011, J. Clin. Oncology 29, Suppl, Abstr #7505). Antibody 5D5 is also described in U.S. Pat. Nos. 6,468,529 and 6,207,152 and in US Patent Application No. 2010/0016241 (Genentech, Inc).

US Patent Application No. 2010/0129369 (Eli Lilly & Co) also describes anti-MET antibodies that bind within the MET Sema domain.

European Patent Application EP2014681 (Pierre Fabre Medicament) describes three anti-MET antibodies with antagonistic activity in a bivalent format. One of these antibodies binds within the Sema domain of MET, whereas the other two antibodies bind the extracellular region of MET outside the Sema domain.

The anti-MET antibody DN-30 causes MET activation and shedding through ADAM10 (Petrelli et al, 2006; Schelter et al, 2010). Conversion of the intact IgG into a monovalent format abolished agonistic activity and yielded a bona fide antagonist (Pacchiana et al, 2010). According to US patent application No. 2009/0285807, antibody DN-30 binds to the IPT4 region of MET.

Nevertheless, there exists a need for further antagonist antibodies to human MET.

The inventors have generated scFv antibodies with antagonistic activity against human MET. Antibody 7A2 was selected for further study as it had a high affinity for MET, bound a distinct epitope from 5D5 and DN-30 on the MET extracellular domain, and was a strong inhibitor of MET activity. This antibody was mutated to identify further useful antibodies with improved activities, and the sequences of antibody 7A2, and the improved variants, have been obtained.

Accordingly, a first aspect of the invention provides an antibody that specifically binds to the extracellular domain of human MET (SEQ ID No: 1), wherein the antibody comprises:
a heavy chain CDR1 comprising the amino acid sequence DYYMH (SEQ ID No: 2), or a variant thereof comprising 1, 2 or 3 amino acid substitutions,
a heavy chain CDR2 comprising the amino acid sequence LVDPEDGETIYAEKFQ (SEQ ID No: 3), or a variant thereof comprising 1, 2 or 3 amino acid substitutions, and
a heavy chain CDR3 comprising the amino acid sequence DATTPYYGMDV (SEQ ID No: 4), or a variant thereof wherein
   the Y at position 7 is replaced with W,
   the G at position 8 is replaced with F,
   the M at position 9 is replaced with P,
   the D at position 10 is replaced with M, W, V, Q, R or Y, and/or
   the V at position 11 is replaced with E, W, Q, S, T, L, E or L, or
   a combination thereof.

For the avoidance of any doubt, the amino acid positions within this heavy chain CDR3 sequence are numbered consecutively from left to right, i.e. D is at position 1, A is at position 2 . . . and V is at position 11.

Thus, in this aspect of the invention, the variant may have any combination of Y or W at position 7; G or F at position 8; M or P at position 9; D, M, W, V, Q, R or Y at position 10; and V, E, W, Q, S, T, L, E or L at position 11.

In an embodiment, one of the D, A, T, T, P and Y at position 1-6 respectively may also have been replaced with another amino acid.

An alternative embodiment of this aspect of the invention provides an antibody that specifically binds to the extracellular domain of human MET, wherein the antibody comprises:
a heavy chain CDR1 comprising the amino acid sequence DYYMH (SEQ ID No: 2), or a variant thereof comprising 1, 2 or 3 amino acid substitutions,
a heavy chain CDR2 comprising the amino acid sequence LVDPEDGETIYAEKFQ (SEQ ID No: 3), or a variant thereof comprising 1, 2 or 3 amino acid substitutions, and
a heavy chain CDR3 comprising the amino acid sequence DATTPYYGMDV (SEQ ID No: 4), DATTPYWGMVE (SEQ ID No: 5), DATTPYWGMMW (SEQ ID No: 6), DATTPYWGMWQ (SEQ ID No: 7), DATTPYWGMVS (SEQ ID No: 8), DATTPYWGMQT (SEQ ID No: 9), DATTPYWGMQL (SEQ ID No: 10), DATTPYWFPRE (SEQ ID No: 11), or DATTPYWFPYL (SEQ ID No: 12), or a variant of any of these sequences comprising 1, 2 or 3 amino acid substitutions.

A further alternative embodiment of this aspect of the invention provides an antibody that specifically binds to the extracellular domain of human MET, wherein the antibody comprises a heavy chain CDR3 comprising the amino acid sequence DATTPYYGMDV (SEQ ID No: 4), DATTPYWGMVE (SEQ ID No: 5), DATTPYWGMMW (SEQ ID No: 6), DATTPYWGMWQ (SEQ ID No: 7), DATTPYWGMVS (SEQ ID No: 8), DATTPYWGMQT (SEQ ID No: 9), DATTPYWGMQL (SEQ ID No: 10), DATTPYWFPRE (SEQ ID No: 11) or DATTPYWFPYL (SEQ ID No: 12). The antibody may also comprise a heavy chain CDR1 comprising SEQ ID No: 2, or a variant thereof, and/or a heavy chain CDR2 comprising SEQ ID No: 3, or a variant thereof, as described above.

It is appreciated that molecules containing three or fewer CDR regions (in some cases, even just a single CDR or a part thereof) are capable of retaining the antigen-binding activity of the antibody from which the CDR(s) are derived. For example, Gao et al (1994, J. Biol. Chem., 269: 32389-93) describe a whole V_{L} chain (including all three CDRs) having high affinity for its substrate.

Molecules containing two CDR regions are described, for example, by Vaughan & Sollazzo (2001, Combinatorial Chemistry & High Throughput Screening, 4: 417-430). On page 418 (right column—3 Our Strategy for Design) a minibody including only the H1 and H2CDR hypervariable regions interspersed within framework regions is described. The minibody is described as being capable of binding to a target. Pessi et al (1993, Nature, 362: 367-9) and Bianchi et al (1994, J. Mol. Biol., 236: 649-59) are referenced by Vaughan & Sollazzo and describe the H1 and H2 minibody and its properties in more detail. Qiu et al (2007, Nature Biotechnology, 25:921-9) demonstrate that a molecule consisting of two linked CDRs are capable of binding antigen (abstract and page 926, right-hand column). Quiocho (1993, Nature, 362: 293-4) provides a summary of the Pessi et al. “minibody” technology. Ladner (2007, Nature Biotechnology, 25:875-7) reviews the Qiu et al. article and comments that molecules containing two CDRs are capable of retaining antigen-binding activity (page 875, right-hand column).

Molecules containing a single CDR region are described, for example, by Laune et al (1997, JBC, 272: 30937-44) who demonstrate that a range of hexapeptides derived from a CDR display antigen-binding activity (abstract) and note that synthetic peptides of a complete, single, CDR display strong binding activity (page 30942, right-hand column). Monnet et al (1999, JBC, 274: 3789-96) show that a range of 12-mer peptides and associated framework regions have antigen-binding activity (abstract) and comment that a CDR3-like peptide alone is capable of binding antigen (page 3785, left-hand column). Heap et al (2005, J. Gen. Virol., 86: 1791-1800) report that a “micro-antibody” (a molecule containing a single CDR) is capable of binding antigen (abstract and page 1791, left-hand column) and shows that a cyclic peptide from an anti-HIV antibody has antigen-binding activity and function. Nicaise et al (2004, Protein Science, 13:1882-91) show that a single CDR can confer antigen-binding activity and affinity for its lysozyme antigen.

This aspect of the invention also provides an antibody that specifically binds to the extracellular domain of human MET, wherein the antibody comprises:
a light chain CDR1 comprising the amino acid sequence QASQDISNYLN (SEQ ID No: 13),
a light chain CDR2 comprising the amino acid sequence DASNLET (SEQ ID No: 14), and
a light chain CDR3 comprising the amino acid sequence QQGDSFPLT (SEQ ID No: 15),
   or a variant of any of these sequences comprising 1, 2 or 3 amino acid substitutions.

In an alternative embodiment, this aspect of the invention further provides an antibody that specifically binds to the extracellular domain of human MET, wherein the antibody comprises a light chain CDR3 comprising the amino acid sequence QQGDSFPLT (SEQ ID No: 15). The antibody may also comprise a light chain CDR1 comprising SEQ ID No: 13, or a variant thereof, and/or a light chain CDR2 comprising SEQ ID No: 14, or a variant thereof, as described above.

In an embodiment, the antibody may comprise both light and heavy chain CDRs as described above.

In an embodiment, the antibody that specifically binds to the extracellular domain of human MET may comprise a heavy chain variable region comprising an amino acid sequence selected from:

(SEQ ID No: 16)
QMQLVQSGAEVKKPGAPVKVSCKVSGYTFTDYYMHWVQQAPGKGL
EWMGLVDPEDGETIYAEKFQGRVTITADTSTDTAYMELSSLRSED
TAVYYCATDATTPYYGMDVWGQGTLVTVSS;
(SEQ ID No: 17)
QMQLVQSGAEVKKPGAPVKVSCKVSGYTFTDYYMHWVQQAPGKGL
EWMGLVDPEDGETIYAEKFQGRVTITADTSTDTAYMELSSLRSED
TAVYYCATDATTPYWGMVEWGQGTLVTVSS;
(SEQ ID No: 18)
QMQLVQSGAEVKKPGAPVKVSCKVSGYTFTDYYMHWVQQAPGKGL
EWMGLVDPEDGETIYAEKFQGRVTITADTSTDTAYMELSSLRSED
TAVYYCATDATTPYWGMMWWGQGTLVTVSS;
(SEQ ID No: 19)
QMQLVQSGAEVKKPGAPVKVSCKVSGYTFTDYYMHWVQQAPGKGL
EWMGLVDPEDGETIYAEKFQGRVTITADTSTDTAYMELSSLRSED
TAVYYCATDATTPYWGMWQWGQGTLVTVSS;
(SEQ ID No: 20)
QMQLVQSGAEVKKPGAPVKVSCKVSGYTFTDYYMHWVQQAPGKGL
EWMGLVDPEDGETIYAEKFQGRVTITADTSTDTAYMELSSLRSED
TAVYYCATDATTPYWGMVSWGQGTLVTVSS;
(SEQ ID No: 21)
QMQLVQSGAEVKKPGAPVKVSCKVSGYTFTDYYMHWVQQAPGKGL
EWMGLVDPEDGETIYAEKFQGRVTITADTSTDTAYMELSSLRSED
TAVYYCATDATTPYWGMQTWGQGTLVTVSS;
(SEQ ID No: 22)
QMQLVQSGAEVKKPGAPVKVSCKVSGYTFTDYYMHWVQQAPGKGL
EWMGLVDPEDGETIYAEKFQGRVTITADTSTDTAYMELSSLRSED
TAVYYCATDATTPYWGMQLWGQGTLVTVSS;
(SEQ ID No: 23)
QMQLVQSGAEVKKPGAPVKVSCKVSGYTFTDYYMHWVQQAPGKGL
EWMGLVDPEDGETIYAEKFQGRVTITADTSTDTAYMELSSLRSED
TAVYYCATDATTPYWFPREWGQGTLVTVSS;
and
(SEQ ID No: 24)
QMQLVQSGAEVKKPGAPVKVSCKVSGYTFTDYYMHWVQQAPGKGL
EWMGLVDPEDGETIYAEKFQGRVTITADTSTDTAYMELSSLRSED
TAVYYCATDATTPYWFPYLWGQGTLVTVSS,

or a variant of any of these sequences comprising 1, 2, 3, 4 or 5 amino acid substitutions.

Additionally or alternatively (i.e., optionally in combination with one of the heavy chain sequences of SEQ ID Nos: 16-24, or variants thereof, as described above), the antibody may comprise the light chain variable region sequence: DIQMTQSPSSLSASVGDRVTITCQASQDISNYLNWYQQKPGRAPKVLIYDASNLETGVP SRFSGSGSGTEFTLTISNLRPDDFATYYCQQGDSFPLTFGGGTKVEIK (SEQ ID No: 25), or a variant thereof comprising 1, 2, 3, 4 or 5 amino acid substitutions.

Typically, it is preferred that the amino acid substitutions are conservative amino acid substitutions. Conservative amino acid substitutions are well known in the art and include (original residue [symbol] Substitution) Ala (A) [symbol] Val, Gly or Pro; Arg (R) [symbol] Lys or His; Asn (N) [symbol] Gln; Asp (D) [symbol] Glu; Cys (C) [symbol] Ser; Gln (Q) [symbol] Asn; Glu (G) [symbol] Asp; Gly (G) [symbol] Ala; His (H) [symbol] Arg; Ile (I) [symbol] Leu; Leu (L) [symbol] Ile, Val or Met; Lys (K) [symbol] Arg; Met (M) [symbol] Leu; Phe (F) [symbol] Tyr; Pro (P) [symbol] Ala; Ser (S) [symbol] Thr or Cys; Thr (T) [symbol] Ser; Trp (W) [symbol] Tyr; Tyr (Y) [symbol] Phe or Trp; and Val (V) [symbol] Leu or Ala.

In a specific embodiment, the antibody that specifically binds to the extracellular domain of human MET may comprise the amino acid sequence of antibody 7A2 scFv:

(SEQ ID No: 26)
QMQLVQSGAEVKKPGAPVKVSCKVSGYTFTDYYMHWVQQAPGKGL
EWMGLVDPEDGETIYAEKFQGRVTITADTSTDTAYMELSSLRSED
TAVYYCATDATTPYYGMDVWGQGTLVTVSSLEGGGGSGGGGSGGG
ASDIQMTQSPSSLSASVGDRVTITCQASQDISNYLNWYQQKPGRA
PKVLIYDASNLETGVPSRFSGSGSGTEFTLTISNLRPDDFATYYC
QQGDSFPLTFGGGTKVEIK.

This aspect of the invention also includes an antibody that competes with antibody 7A2 scFv (SEQ ID No: 26) specific binding to the extracellular domain of human MET.

This aspect of the invention also includes an antibody that competes with an antibody comprising a heavy chain variable region sequence selected from SEQ ID Nos: 16-24, and a light chain variable region sequence of SEQ ID No: 25, for specific binding to the extracellular domain of human MET.

In a further embodiment, the antibody that specifically binds to the extracellular domain of human MET may be a (7A2) Fab comprising the heavy chain amino acid sequence of SEQ ID No: 28 and/or the light chain amino acid sequence of SEQ ID No: 29, and in a more preferred embodiment, both of these sequences.

The invention also includes an antibody that competes with antibody 7A2 Fab comprising the heavy chain amino acid sequence of SEQ ID No: 28 and the light chain amino acid sequence of SEQ ID No: 29 for specific binding to the extracellular domain of human MET.

In a still further embodiment, the antibody that specifically binds to the extracellular domain of human MET may be a (107_A07) Fab comprising the heavy chain amino acid sequence of SEQ ID No: 30 and/or the light chain amino acid sequence of SEQ ID No: 31, and in a more preferred embodiment, both of these sequences.

The invention also includes an antibody that competes with antibody 107_A07 Fab comprising the heavy chain amino acid sequence of SEQ ID No: 30 and the light chain amino acid sequence of SEQ ID No: 31 for specific binding to the extracellular domain of human MET.

In a still further embodiment, the antibody that specifically binds to the extracellular domain of human MET may be a bivalent IgG2 formatted (107_A07) antibody comprising the heavy chain amino acid sequence of SEQ ID No: 32 and/or the light chain amino acid sequence of SEQ ID No: 31, and in a more preferred embodiment, both of these sequences.

The invention also includes an antibody that competes with a bivalent IgG2 formatted (107_A07) antibody comprising the heavy chain amino acid sequence of SEQ ID No: 32 and the light chain amino acid sequence of SEQ ID No: 31 for specific binding to the extracellular domain of human MET.

It is preferred that the antibodies of this aspect of the invention specifically bind to human MET (SEQ ID No: 1) within amino acid residues 568-741.

It is also preferred that an antibody of this aspect of the invention is an antagonist of human MET, as described below.

The sequence of human MET, taken from Swiss-Prot Accession No. P08581.4, version dated 22 Oct. 2011, is as follows:

(SEQ ID No: 1)

| | |
|---|---|
| 1 | MKAPAVLAPG ILVLLFTLVQ RSNGECKEAL AKSEMNVNMK |
| | YQLPNFTAET PIQNVILHEH |
| 61 | HIFLGATNYI YVLNEEDLQK VAEYKTGPVL EHPDCFPCQD |
| | CSSKANLSGG VWKDNINMAL |
| 121 | VVDTYYDDQL ISCGSVNRGT CQRHVFPHNH TADIQSEVHC |
| | IFSPQIEEPS QCPDCVVSAL |
| 181 | GAKVLSSVKD RFINFFVGNT INSSYFPDHP LHSISVRRLK |
| | ETKDGFMFLT DQSYIDVLPE |
| 241 | FRDSYPIKYV HAFESNNFIY FLTVQRETLD AQTFHTRIIR |
| | FCSINSGLHS YMEMPLECIL |
| 301 | TEKRKKRSTK KEVFNILQAA YVSKPGAQLA RQIGASLNDD |
| | ILFGVFAQSK PDSAEPMDRS |
| 361 | AMCAFPIKYV NDFFNKIVNK NNVRCLQHFY GPNHEHCFNR |
| | TLLRNSSGCE ARRDEYRTEF |
| 421 | TTALQRVDLF MGQFSEVLLT SISTFIKGDL TIANLGTSEG |
| | RFMQVVVSRS GPSTPHVNFL |
| 481 | LDSHPVSPEV IVEHTLNQNG YTLVITGKKI TKIPLNGLGC |
| | RHFQSCSQCL SAPPFVQCGW |
| 541 | CHDKCVRSEE CLSGTWTQQI CLPAIYKVFP NSAPLEGGTR |
| | LTICGWDFGF RRNNKFDLKK |
| 601 | TRVLLGNESC TLTLSESTMN TLKCTVGPAM NKHFNMSIII |
| | SNGHGTTQYS TFSYVDPVIT |
| 661 | SISPKYGPMA GGTLLTLTGN YLNSGNSRHI SIGGKTCTLK |
| | SVSNSILECY TPAQTISTEF |
| 721 | AVKLKIDLAN RETSIFSYRE DPIVYEIHPT KSFISGGSTI |
| | TGVGKNLNSV SVPRMVINVH |
| 781 | EAGRNFTVAC QHRSNSEIIC CTTPSLQQLN LQLPLKTKAF |
| | FMLDGILSKY FDLIYVHNPV |
| 841 | FKPFEKPVMI SMGNENVLEI KGNDIDPEAV KGEVLKVGNK |
| | SCENIHLHSE AVLCTVPNDL |
| 901 | LKLNSELNIE WKQAISSTVL GKVIVQPDQN FTGLIAGVVS |
| | ISTALLLLLG FFLWLKKRKQ |
| 961 | IKDLGSELVR YDARVHTPHL DRLVSARSVS PTTEMVSNES |
| | VDYRATFPED QFPNSSQNGS |
| 1021 | CRQVQYPLTD MSPILTSGDS DISSPLLQNT VHIDLSALNP |
| | ELVQAVQHVV IGPSSLIVHF |
| 1081 | NEVIGRGHFG CVYHGTLLDN DGKKIHCAVK SLNRITDIGE |
| | VSQFLTEGII MKDFSHPNVL |
| 1141 | SLLGICLRSE GSPLVVLPYM KHGDLRNFIR NETHNPTVKD |
| | LIGFGLQVAK GMKYLASKKF |
| 1201 | VHRDLAARNC MLDEKFTVKV ADFGLARDMY DKEYYSVHNK |
| | TGAKLPVKWM ALESLQTQKF |
| 1261 | TTKSDVWSFG VLLWELMTRG APPYPDVNTF DITVYLLQGR |
| | RLLQPEYCPD PLYEVMLKCW |
| 1321 | HPKAEMRPSF SELVSRISAI FSTFIGEHYV HVNATYVNVK |
| | CVAPYPSLLS SEDNADDEVD |
| 1381 | TRPASFWETS. |

Typically and preferably, by human MET we refer to the human polypeptide whose sequence is listed above. However, by human MET we also include isoforms and variants of the human MET protein, including those disclosed in Swiss-Prot Accession No. P08581.4, version dated 22 Oct. 2011, which is incorporated herein in its entirety by reference, unless the context demands otherwise.

By “antibody” we include substantially intact antibody molecules, as well as antigen-binding fragments of antibodies, chimaeric antibodies, humanised antibodies, human antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments and derivatives thereof.

The term “antibody” includes all classes of antibodies, including IgG, IgA, IgM, IgD and IgE. Thus, the antibody may be an IgG molecule, such as an IgG1, IgG2, IgG3, or IgG4 molecule.

In one embodiment, the antibody is an IgG antibody, for example, an IgG2 or IgG4 antibody. In one embodiment, the antibody is an IgG4 antibody in which the Serine amino acid at position 241 has been substituted with a Proline residue (i.e. S241P)—such a substitution is known to stabilise the disulphide bridges in IgG4 molecule, resulting in a more stable antibody (Angal et al., 1993, Mol. Immunol., 30:105-8).

It will be appreciated by persons skilled in the art that the binding specificity of an antibody or antigen binding fragment thereof is conferred by the presence of Complementarity Determining Regions (CDRs) within the variable regions of the constituent heavy and light chains. The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent-parented antibody (Morrison et al (1984) Proc. Natl. Acad. Sci. USA 81, 6851-6855).

Antigenic specificity is conferred by variable domains and is independent of the constant domains, as known from experiments involving the expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544), all of which are included in the present invention. A general review of the techniques involved in the synthesis of antibody fragments which retain their specific binding sites is to be found in Winter & Milstein (1991) Nature 349, 293-299.

The term “antibody” includes antigen-binding fragments of a complete antibody, for example a functional fragment of an antibody that is capable of binding to the specified region of human MET. Accordingly, exemplary antibodies of the invention may be selected from the group consisting of Fv fragments (e.g. single chain Fv and disulphide-bonded Fv), and Fab-like fragments (e.g. Fab fragments, Fab′ fragments and F(ab)₂ fragments), and single domain antibodies (dAbs).

In a preferred embodiment, the antibody may be a scFv, particularly a monovalent scFV. In an alternative preferred embodiment, the antibody may be a Fab.

In another preferred embodiment, the antibody may possess any of the antibody-like scaffolds described by Carter (2006) “Potent antibody therapeutics by design”, Nat Rev Immunol. 6(5):343-57, and Carter (2011) “Introduction to current and future protein therapeutics: a protein engineering perspective”, Exp Cell Res. 317(9): 1261-9. incorporated herein by reference, together with the specificity determining regions described herein. Thus, the term “antibody” also includes affibodies and non-immunoglobulin based frameworks.

The advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli or yeast, thus allowing convenient production in the laboratory and economical production on a commercial scale.

Methods of generating antibodies and antibody fragments are well known in the art. For example, antibodies may be generated via any one of several methods which employ induction of in vivo production of antibody molecules, screening of immunoglobulin libraries (Orlandi et al, 1989, Proc. Natl. Acad. Sci. U.S.A. 86: 3833-3837; Winter et al., 1991, Nature 349: 293-299) or generation of monoclonal antibody molecules by cell lines in culture. These include, but are not limited to, the hybridoma technique, the human B-cell hybridoma technique, and the Epstein-Barr virus (EBV)-hybridoma technique (Kohler et al., 1975. Nature 256:4950497; Kozbor et al., 1985. J. Immunol. Methods 81:31-42; Cote et al., 1983, Proc. Natl. Acad. Sci. USA 80:2026-2030; Cole et al., 1984. Mol. Cell. Biol. 62:109-120).

Methods of producing and identifying anti-MET antibodies are also disclosed in U.S. Pat. No. 6,468,529, U.S. Pat. No. 6,207,152, US 2010/0016241, US 2010/0129369, and EP 2014681, incorporated herein by reference.

The antibody may be produced by recombinant means.

It has previously been shown that bivalent antibody formats may lead to agonism by causing association/dimerisation of receptor molecules. As such monovalent antibody formats may in some cases be advantageous. For example the Genentech 5D5 antibody is known to be a MET agonist when in a bivalent format and this led them to develop a single arm monovalent form of the antibody. Thus, in certain embodiments, it is preferred that the antibody according to the invention is a monovalent antibody. Many suitable monovalent antibody formats, and methods for producing them, are known in the art and include, for example, the disclosures in WO 2007/048037 (Amgen) and WO/2007/059782 (Genmab), which are incorporated herein by reference.

Preferably, the antibody is a monoclonal antibody. Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982), which are incorporated herein by reference.

Antibody fragments can also be obtained using methods well known in the art (see, for example, Harlow & Lane, 1988, “Antibodies: A Laboratory Manual”, Cold Spring Harbor Laboratory, New York, which is incorporated herein by reference). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods. Alternatively, antibody to fragments can be obtained by cell-free in vitro expression, as is known in the art.

In other embodiments, the antibody may be a single-domain antibody, such as a Nanobody. Such antibodies are known to exist in camelids (Curr. Opin. Pharmacol., 8, (2008), 600-608) and sharks (e.g. IgNAR; Curr. Opin. Pharmacol., 8, (2008), 600-608). Nanobodies® are antibody-derived therapeutic proteins that contain the structural and functional properties of naturally-occurring heavy-chain antibodies. The Nanobody® technology was developed following the discovery that camelidae (camels and llamas) possess fully functional antibodies that lack light chains. These heavy-chain antibodies contain a single variable domain (VHH) and two constant domains (C_{H}2 and C_{H}3). The cloned and isolated VHH domain is a perfectly stable polypeptide harbouring the full antigen-binding capacity of the original heavy-chain antibody. These VHH domains with their unique structural and functional properties form the basis of Nanobodies®. They combine the advantages of conventional antibodies (high target specificity, high target affinity and low inherent toxicity) with important features of small molecule drugs (the ability to inhibit enzymes and access receptor clefts). Furthermore, they are stable, have the potential to be administered by means other than injection, are easier to manufacture, and can be humanised. (See, for example U.S. Pat. No. 5,840,526; U.S. Pat. No. 5,874,541; U.S. Pat. No. 6,005,079, U.S. Pat. No. 6,765,087; EP 1589107; WO 97/34103; WO 97/49805; U.S. Pat. No. 5,800,988; U.S. Pat. No. 5,874,541 and U.S. Pat. No. 6,015,695).

Other preferred antibodies include isolated heavy-chain variable (V_{H}) regions or isolated light-chain (V_{L}) regions, for example from human antibodies (Curr. Opin. Pharmacol., 8, (2008), 600-608), and iMabs (WO 03/050283).

Also included within the scope of the invention are modified versions of antibodies and antigen-binding fragments thereof, e.g. modified by the covalent attachment of polyethylene glycol (PEG) or other suitable polymers.

The antibody or antigen-binding fragment of the invention may comprise one or more amino acids which have been modified or derivatised. For example, chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be appreciated that the antibody (i.e., including the antigen-binding fragment) may conveniently be blocked at its N- or C-terminus so as to help reduce susceptibility to exo-proteolytic digestion.

A variety of un-coded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, which are incorporated herein by reference.

A common theme among many of the synthetic strategies has been the introduction of some cyclic moiety into a peptide-based framework. The cyclic moiety restricts the conformational space of the peptide structure and this frequently results in an increased specificity of the peptide for a particular biological receptor. An added advantage of this strategy is that the introduction of a cyclic moiety into a peptide may also result in the peptide having a diminished sensitivity to cellular peptidases. Thus, exemplary antibody, or antigen-binding fragment thereof may comprise terminal modifications as is known in the art, to reduce susceptibility by proteinase digestion and therefore to prolong their half-life in biological fluids where proteases may be present.

Advantageously, the antibody or antigen-binding fragment thereof may be human or humanised.

It will be appreciated by persons skilled in the art that, for human therapy or diagnostics, humanised antibodies may be used. Humanised forms of non-human (e.g. murine) antibodies are genetically engineered chimaeric antibodies or antibody fragments having minimal-portions derived from non-human antibodies. Humanised antibodies include antibodies in which complementary determining regions of a human antibody (recipient antibody) are replaced by residues from a complementary determining region of a non human species (donor antibody) such as mouse, rat of rabbit having the desired functionality. In some instances, Fv framework residues of the human antibody are replaced by corresponding non-human residues. Humanised antibodies may also comprise residues which are found neither in the recipient antibody nor in the imported Complementarity Determining Region (CDR) or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986. Nature 321:522-525; Riechmann et al., 1988, Nature 332:323-329; Presta, 1992, Curr. Op. Struct. Biol. 2:593-596, which are incorporated herein by reference).

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Nature 321:522-525; Reichmann et al., 1988. Nature 332:323-327; Verhoeyen et al., 1988, Science 239:1534-1536I; and U.S. Pat. No. 4,816,567, which are incorporated herein by reference) by substituting human complementarity determining regions with corresponding rodent complementarity determining regions. Accordingly, such humanised antibodies are chimaeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies.

Completely human antibodies may be produced using recombinant technologies. Typically large libraries comprising billions of different antibodies are used. In contrast to the previous technologies employing chimaerisation or humanisation of e.g. murine antibodies this technology does not rely on immunisation of animals to generate the specific antibody. Instead the recombinant libraries comprise a huge number of pre-made antibody variants wherein it is likely that the library will have at least one antibody specific for any antigen. Thus, using such libraries, an existing antibody having the desired binding characteristics can be identified. In order to find the good binder in a library in an efficient manner, various systems where phenotype i.e. the antibody or antibody fragment is linked to its genotype i.e. the encoding gene have been devised. The most commonly used such system is the so called phage display system where antibody fragments are expressed, displayed, as fusions with phage coat proteins on the surface of filamentous phage particles, while simultaneously carrying the genetic information encoding the displayed molecule (McCafferty et al, 1990, Nature 348: 552-554). Phage displaying antibody fragments specific for a particular antigen may be selected through binding to the antigen in question. Isolated phage may then be amplified and the gene encoding the selected antibody variable domains may optionally be transferred to other antibody formats, such as e.g. full-length immunoglobulin, and expressed in high amounts using appropriate vectors and host cells well known in the art. Alternatively, the “human” antibodies can be made by immunising transgenic mice which contain, in essence, human immunoglobulin genes (Vaughan et al (1998) Nature Biotechnol. 16, 535-539).

The format of displayed antibody specificities on phage particles may differ. The most commonly used formats are Fab (Griffiths et al, 1994. EMBO J. 13: 3245-3260) and single chain (scFv) (Hoogenboom et al, 1992, J Mol Biol. 227: 381-388) both comprising the variable antigen binding domains of antibodies. The single chain format is composed of a variable heavy domain (V_{H}) linked to a variable light domain (V_{L}) via a flexible linker (U.S. Pat. No. 4,946,778). Before use as a therapeutic agent, the antibody may be transferred to a soluble format e.g. Fab or scFv and analysed as such. In later steps the antibody fragment identified to have desirable characteristics may be transferred into yet other formats such as full-length antibodies.

WO 98/32845 and Soderlind et al (2000) Nature BioTechnol. 18: 852-856 describe technology for the generation of variability in antibody libraries. Antibody fragments derived from this library all have the same framework regions and only differ in their CDRs. Since the framework regions are of germline sequence the immunogenicity of antibodies derived from the library, or similar libraries produced using the same technology, are expected to be particularly low (Soderlind et al, 2000). This property is of great value for therapeutic antibodies, reducing the risk that the patient forms antibodies to the administered antibody, thereby reducing risks for allergic reactions, the occurrence of blocking antibodies, and allowing a long plasma half-life of the antibody. Thus, when developing therapeutic antibodies to be used in humans, modern recombinant library technology (Soderlind et al, 2001, Comb. Chem. & High Throughput Screen. 4: 409-416) is often used in preference to the earlier hybridoma technology.

Further useful methods for obtaining the desired antibodies are described by Schofield et al, 2007, Genome Biol. 8(11); Pershad et al, 2010, Protein Engineering, Design and Selection 23(4): 279-288; and Dyson et al, 2011, 417: 25-35, which are incorporated herein by reference.

Once suitable antibodies are obtained, they may be tested for activity, such as binding specificity or a biological activity of the antibody, for example by ELISA, immunohistochemistry, flow cytometry, immunoprecipitation, Western blots, etc. The biological activity may be tested in different assays with readouts for that particular feature.

In a preferred embodiment, the antibody, or antigen-binding fragment, is in an isolated and/or purified form.

In preferred embodiments, the antibody is a non-naturally occurring antibody. Of course, where the antibody is a naturally occurring antibody, it is provided in an isolated form (i.e. distinct from that in which it is found in nature).

Prediction of the extracellular domain structure of MET indicates that MET shares homology with semaphorins and plexins. The N-terminus of MET contains a Sema domain of approximately 500 amino acids that is conserved in all semaphorins and plexins. The semaphorins and plexins belong to a large family of secreted and membrane-bound proteins first described for their role in neural development. However, more recently, semaphorin overexpression has been correlated with tumour invasion and metastasis. A cysteine-rich PSI domain (also referred to as a Met Related Sequence domain) found in plexins, semaphorins, and integrins lies adjacent to the Sema domain, followed by four IPT repeats that are immunoglobulin-like regions found in plexins and transcription factors. As shown in FIG. 1, the IPT1 region is located at about residues 562-652), the IPT2 region of MET is located at about residues 653-734, the IPT3 region of MET is located at about residues 735-834, and the IPT4 region of MET is located at about residues 835-922. The MET Sema domain is sufficient for HGF and heparin binding (Gherardi et al, 2003), and the IPT3/4 domains have been reported to contain a ligand binding site (Basilico et al, 2008, J. Biol. Chem. 283: 21267-77), whereas the IPT1 and IPT2 domains are not known to contain HGF binding sites.

We have shown that antibody 7A2 does not bind to the Sema domain of MET, but binds to residues 568-741 of human MET, corresponding to the IPT1 (IG1) and IPT2 (IG2) regions. Since the IPT1/2 regions were not known to contain an HGF binding site, it is a surprising location for an antagonistic antibody to bind.

Accordingly, a second aspect of the invention provides an antibody which is an antagonist of the human HGF receptor (MET), wherein the antibody specifically binds within amino acid residues 568-741 of human MET (SEQ ID No: 1).

The antibody binds to residues 568-741 of human MET (SEQ ID No: 1). In other words, the antibody binds within the first two of the IPT domains in the stalk region of MET. Thus, it is appreciated that the antibody may bind to the IPT1 region, or to the IPT2 region of MET or to both the IPT1 and IPT2 regions of MET.

In an embodiment, the antibody of this aspect of the invention may comprise heavy and/or light chain CDRs, or heavy and/or light chain sequences, as described above in the first aspect of the invention.

We have also shown that antibody 7A2 competes with the major binding fragment of HGF, the “NK1” fragment, for binding to MET. This is unexpected as there is no reported binding site for NK1 within the IPT1/2 region of human MET. Indeed, to our knowledge this is the first report of an antibody that can compete with the NK1 fragment of HGF for binding to MET.

The entire HGF/SF binding site has previously been reported to locate to the SEMA domain (Gherardi et al, 2003), and a binding site in the IPT3/4 region has also been reported (Basilico et al, 2008). Binding of the NK1 fragment to the SEMA/PSI region of MET has also been demonstrated (Holmes at al, 2007). Without wishing to be bound by theory, this binding of NK1 to the SEMA/PSI region could explain why antibodies 7A2 and 107_A07 (which binds MET567-741) may interfere with NK1 fragment binding. The inhibition of NK1 binding in the presence of the antibody could be caused by steric or allosteric interactions which could reduce the ability of the NK1 fragment to bind to MET.

Accordingly, a third aspect of the invention provides an antibody which is an antagonist of the human HGF receptor (MET), wherein the antibody competes with the NK1 fragment of human HGF/SF for binding to MET.

The amino acid of the NK1 fragment of HGF/SF as expressed in P. pastoris and used in the Examples is:

(SEQ ID No: 27)
EAEAYVE GQRKRRNTIH EFKKSAKTTL IKIDPALKIK
TKKVNTADQC ADRCTRNKGL PFTCKAFVFD KARKQCLWFP
FNSMSSGVKK EFGHEFDLYE NKDYIRNCII GKGRSYKGTV
SITKSGIKCQ PWSSMIPHEH SFLPSSYRGK DLQENYCRNP
RGEEGGPWCF TSNPEVRYEV CDIPQCSEVE.

However, it is appreciated that the first 2, or the first 4, amino acids of this NK1 sequence can be removed.

By an antibody is an antagonist of MET we mean that the antibody inhibits (i.e., reduces or prevents, or abolishes) one or more activities or functions of MET. Methods for determining whether or not a given antibody is an antagonist of MET are very well known to the person of skill in the art, and include measuring inhibition of MET signalling, inhibition of MET phosphorylation, inhibition of MET kinase activity, inhibition of HGF-dependent cellular proliferation and inhibition of HGF-dependent cellular migration.

Cell signalling can be assessed by a variety of methods and based on a variety of criteria, which are known in the art. For example, occurrence of cell signalling in the HGF/MET pathway can manifest biologically in the form of change in phosphorylation of target molecules in the signalling pathway. Thus, e.g., the amount of protein phosphorylation associated with one or more known phosphorylation targets in the HGF/MET pathway could be measured. Examples of such phosphorylation targets include MET itself and mitogen activated protein kinase (MAPK).

An example of a method of measuring migration is described in Example 7 below. A further example is described by Corps et al (1997) “Hepatocyte growth factor stimulates motility, chemotaxis and mitogenesis in ovarian carcinoma cells expressing high levels of c-met.” Int. J. Cancer 73(1): 151-5, incorporated herein by reference.

In an embodiment, the antibody of this third aspect of the invention may comprise heavy and/or light chain CDRs, or heavy and/or light chain sequences, as described above in the first aspect of the invention.

By crystallising antibody 107_A07 in complex with c-MET (see Example 9), we have been able to show that the antibody binds to a region of human MET defined by amino acids 595-615, which is within the IPT1 (IG1) region.

Accordingly, a fourth aspect of the invention provides an antibody that specifically binds to an epitope located within amino acid residues 592-615 of human MET (SEQ ID No: 1). In other words, the antibody may specifically bind to an epitope comprising one or more amino acids on human MET between residues 592 and 615.

It is appreciated that the antibody may bind to amino acid residues outside of the region of human MET that is defined by residues 592-615. However, the interaction between the antibody and such amino acids is expected to be weaker than that between the antibody and amino acids within the region of MET defined by residues 592-615.

In an embodiment, the antibody is one that does not interact with, or makes only weak interactions with, amino acids outside the region defined by amino acid residues 592-615 of human MET. For example, mutating amino acid residues other than residues 592-615 would not be expected to significantly affect antibody binding (eg reduce it to less than 90%, 80%, 70%, 60% or 50% of the original level of binding).

In an embodiment, the antibody is one that does not compete with an antibody known to bind elsewhere within human MET for specific binding to an epitope located within amino acid residues 592-615 of human MET.

The crystal structure has revealed that the molecular interactions between MET and the antibody are at amino acid positions 592, 593, 595, 599, 600, 602, 611, 613, 614 and 615 of human MET, and so in an embodiment, the antibody is one that binds to an epitope that comprises one or more amino acids at positions 592, 593, 595, 599, 600, 602, 611, 613, 614, 615 of human MET, such as 2 or 3 or 4 or 5 or 6 or 7 or 8 or 9 or 10 of these amino acids.

It will be appreciated that some of the residues identified in the crystal structure may be more important than others. For example, the interactions between the antibody and amino acids T611 and T613 of human MET are relatively long hydrogen bonds (4.88 Å and 3.39 Å, respectively), such that they may be less determinative for antibody binding. Thus, the antibody may be one that binds to an epitope that comprises amino acids at positions 592, 593, 595, 599, 600, 602, 614, and 615 of human MET. Optionally, the epitope further comprises amino acids at positions 611 and/or 613 of human MET.

In human MET, the amino acids at these positions are typically R592, N593, K595, K599, K600, R602, T611, T613, L614 and S615, and so in a preferred embodiment, the antibody is one that binds to an epitope that comprises one or more of amino acids R592, N593, K595, K599, K600, R602, T611, T613, L614 and S615 of human MET, such as 2 or 3 or 4 or 5 or 6 or 7 or 8 or 9 or 10 of these amino acids. However, it is understood that MET is often mutated in cancer cells, and so the antibody may be one that binds to a corresponding epitope on MET as present on the cancer cells (i.e., the mutated versions of MET). By corresponding epitope we include the meaning of an epitope in a mutated version of MET that comprises one or more amino acids that align with a respective one or more of R592, N593, K595, K599, K600, R602, T611, T613, L614 and S615 of human MET, when the mutated MET and human MET are compared using protein alignment programs such as MacVector and CLUSTALW.

In an embodiment, the antibody is one that binds to an epitope comprising amino acids R592, N593, K595, K599, K600, R602, T611 and S615 of human MET.

In an embodiment, the antibody is one that binds to an epitope comprising amino acids R592, N593, K595, K599, K600, R602, T611, T613, L614 and S615 of human MET.

To determine whether the antibody binds to an epitope that comprises one or more amino acids at the defined positions, typically, those amino acids are mutated in human MET, and binding of the antibody tested before and after mutation, for example using routine binding assays such as ELISA or BIACORE. If binding is absent or significantly reduced (eg less than 80%, 70%, 60%, 50%, 40%, 30% or 20% of the original level of binding) following mutation of a particular amino acid, that amino acid is considered to contribute to antibody binding. In this case, mutation of one or more amino acids at positions 592, 593, 595, 599, 600, 602, 611, 613, 614, 615 of human MET, such as 2 or 3 or 4 or 5 or 6 or 7 or 8 or 9 or all 10 of these amino acids, is expected to abolish or significantly reduce binding of the antibody of this aspect of the invention to human MET. Other experimental and computational epitope mapping methods, as well known in the art, may also be used to assess binding of the antibody of this aspect of the invention to the particular epitope, including X-ray crystallography, Nuclear Magnetic Resonance (NMR) spectroscopy, Hydroged deuterium eXchange Mass Spectrometry (HX-MS) and various competition binding methods.

In an embodiment, the antibody of this aspect of the invention is an antagonist of human MET. Since the IPT1 (IG1) region was not known to contain an HGF binding site, it is a surprising location for an antagonistic antibody to bind.

In an embodiment, the antibody of this aspect of the invention may comprise heavy and/or light chain CDRs, or heavy and/or light chain sequences, as described above in the first aspect of the invention.

In an embodiment, the antibody of this aspect of the invention competes with the NK1 fragment of human HGF/SF for binding to MET.

In addition to the features described above, it may be also preferred that the antibodies of the first, second, third and fourth aspects of the invention have an affinity for MET of at least 10⁻⁷ M and more preferably 10⁻⁸ M, although antibodies with higher affinities may be even more preferred. It is preferred that the antibody binds to the specified region of MET with an affinity at least the same as, or greater than, that of the monoclonal antibody produced by the hybridoma cell line deposited under American Type Culture Collection Accession Number ATCC HB-11895 (hybridoma 5D5.11.6) (“monoclonal antibody 5D5”). It is further preferred that the antibody binds to MET with an affinity at least the same as, or greater than, that of the Fab fragment of monoclonal antibody 5D5. The Fab fragment of monoclonal antibody 5D5 can be produced as described in Example 7 of U.S. Pat. No. 6,027,152 and it was used in the inhibition experiments described in US 2010/0016241, both of which are incorporated herein by reference.

In preferred embodiments, the antibody binds to the specified region of MET with an affinity of at least 7 nM, more preferably at least 5 nM, or at least 2 nM, more preferably at least 1 nM. Typically, such affinities are achieved in a Fab, or related, format. Still preferably, the antibody binds to the specified region of MET with an affinity of at least 0.5 nM, more preferably at least 0.2 nM, or at least 0.1 nM, and most preferably at least 0.07 nM. Antibodies with these higher affinities are typically in an scFv, or related, format.

Additionally or alternatively, it is preferred that the antibodies of the first, second, third and fourth aspects of the invention inhibit human HGF-dependent migration of SKOV-3 cells. Preferably, the antibodies inhibit human HGF-dependent migration of SKOV-3 cells at least to the same extent as the Fab fragment of monoclonal antibody 5D5. A suitable method for measuring HGF-dependent migration of SKOV-3 cells is given below in the Examples. It is still further preferred in some embodiments that the antibodies inhibit human HGF-dependent migration of SKOV-3 cells at least 2×, or more preferably at least 3×, or in further preferred embodiments at least 4×, at least 5×, or at least 10× more than the Fab fragment of monoclonal antibody 5D5. In many instances a direct side-by-side comparison is preferred since slight variations in assay conditions may affect the quantitative results from a migration assay. Nevertheless, since a direct comparison may not always be available, it is preferred that the antibodies inhibit human HGF-dependent migration of SKOV-3 cells with an IC₅₀ of at least 386 nM, more preferably at least 13 nM, still more preferably at least 10 nM and most preferably with an IC₅₀ of at least 7 nM (or, more accurately, at least 7.4 nM), under the assay conditions described in Example 7, below.

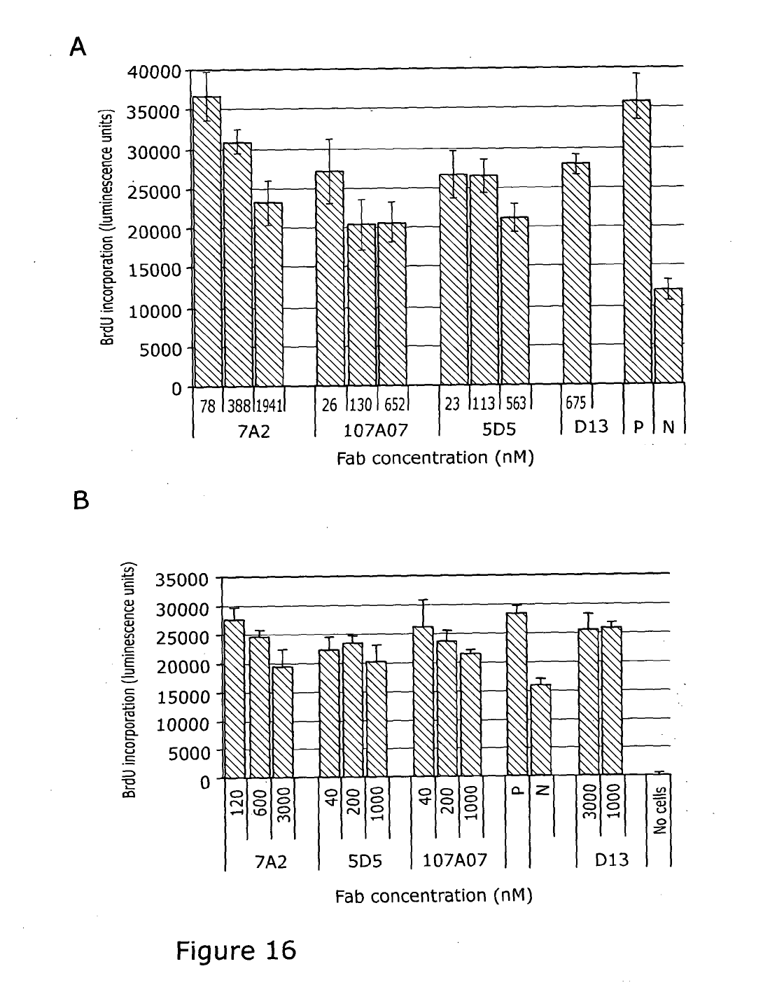

In an embodiment, it is preferred that the antibodies of the first, second, third and fourth aspects of the invention inhibit human HGF-dependent proliferation of BxPC3 cells. A suitable method for measuring HGF-dependent proliferation of BxPC3 cells is given below in the Examples. Preferably, the antibodies inhibit human HGF-dependent proliferation of BxPC3 cells generally to at least to the same extent as the Fab fragment of monoclonal antibody 5D5 as shown in FIG. 16).

In a still further embodiment, it is preferred that the antibodies of the first, second, third and fourth aspects of the invention inhibit human HGF-induced DNA synthesis, and suitable methods for measuring HGF-induced DNA synthesis are well known in the art.

It is appreciated that the anti-MET antibodies of the first three aspects of the invention will bind MET with a greater affinity than for an irrelevant polypeptide, such as human serum albumin (HSA). Preferably, the antibody binds MET with at least 5, or at least 10 or at least 50 times greater affinity than for the irrelevant polypeptide. More preferably, the antibody molecule binds the MET with at least 100, or at least 1,000, or at least 10,000 times greater affinity than for the irrelevant polypeptide. Such binding may be determined by methods well known in the art, such as one of the Biacore® systems.

Preferably, the anti-MET antibodies of the first three aspects of the invention bind human MET with a greater affinity than for the related human receptors RON and PlexinA2. By human RON we mean the polypeptide whose sequence is disclosed in Swiss-Prot Accession No. Q04912.2, version dated 24 Sep. 2011, and by human plexinA2 we mean the polypeptide whose sequence is disclosed in Swiss-Prot Accession No. 075051.4, version dated 22 Oct. 2011. Preferably, the antibody binds MET with at least 5, or at least 10 or at least 50 times greater affinity than for human RON or PlexinA2. More preferably, the antibody molecule binds the MET with at least 100, or at least 1,000, or at least 10,000 times greater affinity than for human RON or PlexinA2. Such binding may be determined by methods well known in the art, such as one of the Biacore® systems.

In an embodiment, when the antibody is administered to an individual, the antibody binds to human MET or to the specified portion thereof, with a greater affinity than for any other molecule in the individual. Preferably, the antibody binds to the specified portion of MET with at least 2, or at least 5, or at least 10 or at least 50 times greater affinity than for any other molecule in the individual. More preferably, the agent binds the MET at the specified domain with at least 100, or at least 1,000, or at least 10,000 times greater affinity than any other molecule in the individual. Preferably, the antibody molecule selectively binds the MET without significantly binding other polypeptides in the body, such as for example other RTKs, although in practice there may be some cross reactivity that does not affect the intended use of the antibody.

Although the antibodies of the invention do not bind within the Sema domain of MET, it may be preferred that binding of the antibody to MET results in disruption of the ability of the Sema domain to interact with its binding partner (such as another MET molecule). In an embodiment, the antibody binds to MET (e.g., at the specified region) such that MET dimerisation is disrupted. Suitably, the ability of the Sema domain to effect MET dimerisation is disrupted. For example, in one embodiment, the invention provides an antibody which upon binding to MET inhibits dimerisation. In an embodiment, an antibody disrupts MET homodimerisation. In an alternative embodiment, the antibody may disrupt MET heterodimerisation (i.e., MET dimerisation with a non-MET molecule). Inhibition of MET dimerisation can be assayed in a variety of ways known in the art, and based on any of a variety of criteria known in the art, e.g., as described in US 2010/0016241.

In some instances, it may be advantageous to have a MET antibody that disrupts both MET dimerisation and ligand binding.

In an embodiment, it may be preferred that the antibodies are able to block ligand independent activation and signalling from MET e.g., as described in US 2010/0016241.

The invention also includes fusions of the antibodies described above. For example, fusion may comprise, consist or consist essentially of an intact antibody. By “consist essentially of” we mean that the fusion thereof consists of a portion of an intact antibody sufficient to retain binding specificity for the specified region of human MET.

By a ‘fusion’ of an antibody we include an antibody or antigen-binding fragment (as defined above) fused to any other moiety, typically a polypeptide. For example, the antibody or antigen-binding fragment may be fused to a polypeptide such as glutathione-S-transferase (GST) or protein A in order to facilitate its purification. Examples of such fusions are well known to those skilled in the art. Similarly, the said antibody or antigen-binding fragment may be fused to an oligo-histidine tag (e.g. His6), a FLAG tag, or to an epitope recognised by a further antibody (such as the well-known Myc tag epitope).

Typically, the fusion comprises a further portion which confers a desirable feature on the antibody or antigen-binding fragment of the invention; for example, the portion may be useful in detecting or isolating the antibody or antigen-binding fragment, or promoting cellular uptake of the antibody or antigen-binding fragment. The portion may be, for example, a biotin moiety, a radioactive moiety, a fluorescent moiety, for example a small fluorophore or a green fluorescent protein (GFP) fluorophore, as well known to those skilled in the art. The moiety may be a lipophilic molecule or polypeptide domain that is capable of promoting cellular uptake, as known to those skilled in the art.

Methods for conjugating additional moieties to an antibody are well known in the art. Exemplary methods are described in Bioconjugate Techniques, 2nd Edition (2008); Hermanson (Academic Press, Inc.) and in Veronese et al., (1999; Farmaco 54(8): 497-516); Stayton et al., (2005; Orthod Craniofac Res 8(3): 219-225); Schrama et al., (2006; Nat Rev Drug Discov 5(2): 147-159); Doronina et al (2003; Nat Biotechnol 21(7): 778-784); Carter et al., (2008; Cancer J 14(3): 154-169); Torchilin (2006; Annu Rev Biomed Eng 8: 343-375); Rihova (1998; Adv Drug Deliv Rev 29(3): 273-289); Goyal et al (2005; Acta Pharm 55(1): 1-25); Chari (1998; Adv Drug Deliv Rev 31(1-2): 89-104); Garnett (2001; Adv Drug Deliv Rev 53(2): 171-216); Allen (2002; Nat Rev Cancer 2(10): 750-763).

In certain embodiments, it is preferred that the antibody has been PEGylated, as is well known in the art. Thus, the invention includes PEGylated antibody fragments such as Fv-PEG, scFv-PEG, Fab-PEG, F(ab′)₂-PEG or Fab′-PEG.

A fifth aspect of the invention provides a nucleic acid molecule encoding the antibody of any of the first three aspects of the invention. The nucleic acid molecule may encode a fusion of the antibody as described above wherein the further non-antibody portion is a polypeptide. The invention includes a vector comprising the nucleic acid molecule, and includes a host cell comprising the nucleic acid molecule or the vector.

The nucleic acid molecule may be DNA or RNA, and is preferably DNA. It may comprise deoxyribonucleotides, ribonucleotides, modified nucleotides or bases, and/or their analogues, or any substrate that can be incorporated into a polymer by DNA or RNA polymerase, or by a synthetic reaction. A polynucleotide may comprise modified nucleotides, such as methylated nucleotides and their analogues. If present, modification to the nucleotide structure may be imparted before or after assembly of the polymer. The sequence of nucleotides may be interrupted by non-nucleotide components. Some specific examples of nucleic acid molecules encoding antibodies of the invention are described below in the Examples. Other suitable sequences can readily be determined based upon the knowledge of antibody structure and the genetic code.

The vector can be of any type, for example a recombinant vector such as an expression vector. The expression vectors contain elements (e.g., promoter, signals of initiation and termination of translation, as well as appropriate regions of regulation of transcription) which allow the expression and/or the secretion of the antibodies in a host cell. Any of a variety of host cells can be used, such as a prokaryotic cell, for example, E. coli, or a eukaryotic cell, for example a mammalian cell such as Chinese Hamster Ovary (CHO) cell, or a yeast, insect or plant cell. Many suitable vectors and host cells are very well known in the art.

It is appreciated that in certain embodiments the nucleic acid molecule and the expression vector may be used in the treatment aspects of the invention via a gene therapy approach using formulations and methods described below and known in the art. However, this treatment approach is not currently preferred.

The invention also includes methods for making an antibody of the invention. For example, the invention comprises expressing in a suitable host cell a recombinant vector encoding the antibody (e.g. an antibody fragment), and recovering the antibody. Methods for expressing and purifying antibodies are very well known in the art.

A sixth aspect of the invention provides a compound comprising an antibody according to any of the first three aspects of the invention and a detectable moiety. The compound may be a fusion as described above.

The detectable moiety may comprise a detectable enzyme such as peroxidase, alkaline phosphatase, beta-D-galactosidase, glucose oxidase, glucose amylase, carbonic anhydrase, acetylcholinesterase, lysozyme, malate dehydrogenase or glucose 6-phosphate dehydrogenase.

The detectable moiety may comprise a molecule such as biotin, digoxygenin or 5-bromodeoxyuridine.

The detectable moiety may comprise a fluorescent moiety (i.e., label) such as fluorescein and its derivatives, fluorochrome, rhodamine and its derivatives, Green Fluorescent Protein (GFP), dansyl, umbelliferone etc. In such conjugates, the antibodies of the invention or their functional fragments can be prepared by methods known to the person skilled in the art. They can be coupled to the enzymes or to the fluorescent labels directly or by the intermediary of a spacer group or of a linking group such as a polyaldehyde, like glutaraldehyde, ethylenediaminetetraacetic acid (EDTA), diethylene-triaminepentaacetic acid (DPTA), or in the presence of coupling agents such as those mentioned above for the therapeutic conjugates. The conjugates containing labels of fluorescein type can be prepared by reaction with an isothiocyanate.

The detectable moiety may comprise a chemiluminescent label such as luminol and the dioxetanes, or a bioluminescent label such as luciferase and luciferin.

The detectable moiety may comprise a radioactive label such as selected from the group consisting of: technetium-99; technetium-99m; iodine-123; iodine-124; iodine-125; iodine-126; iodine-131; iodine-133; indium-111; indium-113m, fluorine-18; fluorine-19; carbon-11; carbon-13; copper-64; nitrogen-13; nitrogen-15; oxygen-15; oxygen-17; arsenic-72; gadolinium; manganese; iron; deuterium; tritium; yttrium-86; zirconium-89; bromine-77, gallium-67; gallium-68, ruthenium-95, ruthenium-97, ruthenium-103, ruthenium-105, mercury-107, rhenium-99m, rhenium-101, rhenium-105, scandium-47. Suitable methods for coupling such radioisotopes to the antibodies—either directly or via a chelating agent such as EDTA or DTPA—can be employed, as is known in the art.

A seventh aspect of the invention provides a compound comprising an antibody according to any of the first three aspects of the invention and a cytotoxic moiety. The compound may be a fusion as described above.

Preferably, the cytotoxic moiety is capable of inhibiting at least one activity of cells expressing MET; more preferably it is capable of preventing the growth or proliferation or migration of the cell; and still more preferably it is capable of totally inactivating or killing the cell.

In an embodiment, the cytotoxic moiety may comprise a radioactive atom. The radioactive atom is typically selected from the group consisting of: iodine-123; iodine-125; iodine-131; indium-111; bromine-77; copper-67; arsenic-77; astatine-211; actinium-225; bismuth-212; bismuth-213; bismuth-217; lutetium-177; holmium-166; phosphorous-33; platinum-193; platinum-195; rhenium-186; rhenium-188; strontium-89; yttrium-90. gold-199, palladium-100; and antimony-211.

The cell toxin may be an enterobacterial toxin, especially Pseudomonas exotoxin A or calicheamicin.

In another embodiment, the cytotoxic moiety may comprise a drug selected from the group consisting of: an alkylating agent (such as cisplatin, carboplatin); an antimetabolite (such as azathioprine, methotrexate); an antimitotic drug (such as vincristine); and a topoisomerase inhibitor (such as doxorubicine, etoposide).

Antibody-drug conjugates for cancer therapy are reviewed by Carter & Senter (2008), Cancer J. 14(3): 154-69, incorporated herein by reference.

In order to facilitate the coupling between the cytotoxic moiety and the antibody, it is possible to directly conjugate the two agents, or to introduce a spacer molecule between them. Suitable spacers include poly(alkylene) glycols such as polyethylene glycol, and peptide linkers. Many suitable coupling techniques are well known in the art. Suitable agents allowing covalent, electrostatic or noncovalent binding of the moiety to the antibody include benzoquinone, carbodiimide and more particularly EDC (1-ethyl-3-[3-dimethyl-aminopropyl]-carbodiimide hydrochloride), dimaleimide, dithiobis-nitrobenzoic acid (DTNB), N-succinimidyl S-acetyl thio-acetate (SATA), the bridging agents having one or more phenylazide groups reacting with the ultraviolets (U.V.) and preferably N-[-4-(azidosalicylamino)butyl]-3′-(2′-pyridyldithio)-propionamide (APDP), N-succinimid-yl 3-(2-pyridyldithio)propionate (SPDP), 6-hydrazino-nicotinamide (HYNIC). Another form of coupling, especially for the radioelements, includes the use of a bifunctional ion chelator. For example, chelates derived from EDTA or DTPA which have been developed for binding metals, especially radioactive metals, and immunoglobulins. Thus, DTPA and its derivatives can be substituted by different groups on the carbon chain in order to increase the stability and the rigidity of the ligand-metal complex, as is well known in the art.

An eighth aspect of the invention provides a pharmaceutical composition comprising an antibody according to any of the first three aspects of the invention, or a nucleic acid molecule or expression vector encoding the antibody, or a compound according to the sixth or seventh aspects of the invention, and a pharmaceutically acceptable diluent, carrier or excipient.

As used herein, ‘pharmaceutical composition’ means a therapeutically effective formulation according to the invention.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce or prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent, carrier or excipient.

In the methods and use for manufacture of compositions of the invention, a therapeutically effective amount of the active component is provided. A therapeutically effective amount can be determined by the ordinary skilled medical worker based on patient characteristics, such as age, weight, sex, condition, complications, other diseases, etc., as is well known in the art.

In the case of cancer, the therapeutically effective amount of the drug has a therapeutic effect and as such may reduce the number of cancer cells; decrease tumorigenicity, tumorigenic frequency or tumorigenic capacity; reduce the number or frequency of cancer stem cells; reduce the tumour size; inhibit or stop cancer cell infiltration into peripheral organs including, for example, the spread of cancer into soft tissue and bone; inhibit and stop tumour metastasis; inhibit and stop tumour growth; relieve to some extent one or more of the symptoms associated with the cancer; reduce morbidity and mortality; or improve quality of life; or a combination of such effects.

By “pharmaceutically acceptable” is included that the formulation is sterile and pyrogen free. Suitable pharmaceutical carriers, diluents and excipients are well known in the art of pharmacy. The carrier(s) must be “acceptable” in the sense of being compatible with the compound and not deleterious to the recipients thereof. Typically, the carriers will be water or saline which will be sterile and pyrogen free; however, other acceptable carriers may be used.

In an embodiment, the pharmaceutical compositions or formulations of the invention are formulated for parenteral administration, more particularly for intravenous administration. In a preferred embodiment, the pharmaceutical composition is suitable for intravenous administration to a patient, for example by injection.

Thus, typically and preferably, the composition is administered intravenously or by intraperitoneal administration to the patient.

Suitably the compound is administered as an infusion or as a bolus injection.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

In an alternative preferred embodiment, the pharmaceutical composition is suitable for topical administration to a patient.

The antibodies, agents, medicaments and pharmaceutical compositions may be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

The antibodies, compounds, medicaments and pharmaceutical compositions of the invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period. Preferably, delivery is performed intra-muscularly and/or sub-cutaneously and/or intravenously.

The antibodies, compounds, medicaments and pharmaceutical compositions of the invention can be administered by a surgically implanted device that releases the drug directly to the required site. For example, Vitrasert releases gancyclovir directly into the eye to treat CMV retinitis. The direct application of this toxic agent to the site of disease achieves effective therapy without the drug's significant systemic side-effects.

Electroporation therapy (EPT) systems can also be employed for the administration of the agents, medicaments and pharmaceutical compositions of the invention. A device which delivers a pulsed electric field to cells increases the permeability of the cell membranes to the drug, resulting in a significant enhancement of intracellular drug delivery.

The antibodies, compounds, medicaments and pharmaceutical compositions of the invention can also be delivered by electro-incorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of delivery of the antibodies, compounds, medicaments and pharmaceutical compositions of the invention is the ReGel injectable system that is thermo-sensitive. Below body temperature, ReGel is an injectable liquid while at body temperature it immediately forms a gel reservoir that slowly erodes and dissolves into known, safe, biodegradable polymers. The active substance is delivered over time as the biopolymers dissolve.

The antibodies, compounds, medicaments and pharmaceutical compositions of the invention can also be delivered orally. The process employs a natural process for oral uptake of vitamin B₁₂ and/or vitamin D in the body to co-deliver proteins and peptides. By riding the vitamin B₁₂ and/or vitamin D uptake system, the antibodies, compounds, medicaments and pharmaceutical compositions of the invention can move through the intestinal wall. Complexes are synthesised between vitamin B₁₂ analogues and/or vitamin D analogues and the drug that retain both significant affinity for intrinsic factor (IF) in the vitamin B₁₂ portion/vitamin D portion of the complex and significant bioactivity of the active substance of the complex.

Preferably, the medicaments and/or pharmaceutical compositions of the present invention are in a unit dosage containing a daily dose or unit, daily sub-dose or an appropriate fraction thereof, of the active ingredient.

The antibodies, compounds, medicaments and pharmaceutical compositions of the invention will normally be administered orally or by any parenteral route, in the form of a pharmaceutical composition comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the antibodies, compounds, medicaments and pharmaceutical compositions of the invention can be administered alone but will generally be administered in admixture with a suitable pharmaceutical excipient, diluent or carrier selected with regard to the intended route of administration and standard pharmaceutical practice.

For example, the antibodies, compounds, medicaments and pharmaceutical compositions of the invention can be administered orally, buccally or sublingually in the form of tablets, capsules, ovules, elixirs, solutions or suspensions, which may contain flavouring or colouring agents, for immediate-, delayed- or controlled-release applications. The antibodies, compounds, medicaments and pharmaceutical compositions of the invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the antibodies, compounds, medicaments and pharmaceutical compositions of the invention may be combined with various sweetening or flavouring agents, colouring matter or dyes, with emulsifying and/or suspending agents and with diluents such as water, ethanol, propylene glycol and glycerin, and combinations thereof.

The antibodies, compounds, medicaments and pharmaceutical compositions of the invention can be administered parenterally, for example, intravenously, intra-arterially, intraperitoneally, intra-thecally, intraventricularly, intrastemally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Medicaments and pharmaceutical compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The medicaments and pharmaceutical compositions may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the antibodies, compounds, medicaments and pharmaceutical compositions of the invention will usually be from 0.002 to 0.4 mg/kg and/or 0.1 mg/kg to 20 mg/kg administered in single or divided doses, and typically at a frequency ranging from two or three times per week to once per month.

Thus, for example, the tablets or capsules of the medicaments and pharmaceutical compositions of the invention may contain from 5 mg to 1400 mg (for example, from 7 mg to 1400 mg, or 5 mg to 1000 mg) and may preferably contain 5 mg to 200 mg of active agent for administration singly or two or more at a time, as appropriate.

The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The antibodies, compounds, medicaments and pharmaceutical compositions of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (FIFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active agent, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of an agent of the invention and a suitable powder base such as lactose or starch. Such formulations may be particularly useful for treating cancers/tumours of the lung, such as, for example, small cell lung carcinoma, non-small cell lung carcinoma, pleuropulmonary blastoma or carcinoid tumour.

Aerosol or dry powder formulations are preferably arranged so that each metered dose or “puff” contains 5 mg to 1400 mg (for example, from 7 mg to 1400 mg, or 5 mg to 1000 mg) and preferably contain 5 mg to 200 mg of an agent of the invention for delivery to the patient. It will be appreciated that the overall daily dose with an aerosol will vary from patient to patient, and may be administered in a single dose or, more usually, in divided doses throughout the day.

Alternatively, the antibodies, compounds, medicaments and pharmaceutical compositions of the invention can be administered in the form of a suppository or pessary, particularly for treating or targeting colon, rectal or prostate tumours.

They may also be applied topically in the form of a lotion, solution, cream, gel, ointment or dusting powder. The antibodies, compounds, medicaments and pharmaceutical compositions of the invention may also be transdermally administered, for example, by the use of a skin patch. They may also be administered by the ocular route. Such formulations may be particularly useful for treating cancers/tumours of the eye, such as retinoblastoma, medulloepithelioma, uveal melanoma, rhabdomyosarcoma, intraocular lymphoma, or orbital lymphoma.

For ophthalmic use, the antibodies, compounds, medicaments and pharmaceutical compositions of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the antibodies, compounds, medicaments and pharmaceutical compositions of the invention can be formulated as a suitable ointment containing the active agent suspended or dissolved in, for example, a mixture with one or more of the following: mineral oil, liquid petrolatum, white petrolatum, propylene glycol, polyoxyethylene polyoxypropylene agent, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water. Such formulations may be particularly useful for treating cancers/tumours of the skin, such as, for example, basal cell cancer, squamous cell cancer or melanoma.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier. Such formulations may be particularly useful for treating cancers/tumours of the mouth and throat.

Generally, in humans, oral or parenteral administration of the medicaments and pharmaceutical compositions comprising the antibody or compound of the invention is the preferred route, being the most convenient.

The antibody or compound of the invention may be formulated at various concentrations, depending on the efficacy/toxicity of the agent being used. For in vitro applications, formulations may comprise a lower concentration than for therapeutic use.

Thus, the present invention provides a pharmaceutical formulation comprising an amount of an antibody or compound of the invention effective to treat various conditions (as described herein).

Preferably, the pharmaceutical composition is adapted for delivery by a route selected from the group comprising: intravenous; intramuscular; subcutaneous; intra-articular; pulmonary; intranasal; intraocular; intrathecal.

The present invention also includes pharmaceutical compositions comprising pharmaceutically acceptable acid or base addition salts of the polypeptide binding moieties of the present invention. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the agents according to the present invention.

The chemical bases that may be used as reagents to prepare pharmaceutically acceptable base salts of the present agents that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

The antibodies and compounds of the invention may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilised (freeze dried) antibody loses no more than about 20%, or no more than about 25%, or no more than about 30%, or no more than about 35%, or no more than about 40%, or no more than about 45%, or no more than about 50% of its activity (prior to lyophilisation) when re-hydrated.

In a further aspect, the invention provides antibodies, nucleic acid molecules or expression vectors encoding the antibody, or compounds of the invention for use in medicine. Methods of manufacturing a medicament using an active agent, such as the antibody, nucleic acid molecule/expression vector or compound of the invention, are well known to persons skilled in the art of medicine and pharmacy.

The invention also includes antibodies, nucleic acid molecules/expression vectors or compounds of the invention for use in medicine.

Another aspect of the invention provides a method of inhibiting activity of human MET on a cell, or of inhibiting human MET-mediated cellular proliferation and/or migration, the method comprising contacting the cell with an effective amount of an antibody according to any of the first three aspects of the invention, or a nucleic acid molecule or expression vector encoding the antibody, or a compound as defined in the seventh aspect of the invention.

Typically, the cell is a cancer or tumour cell, which may be a primary tumour cell, or a cell from an established cell line, for example from one of the cancers/tumours described below.

In an embodiment, the method may be performed on cells in vitro. Alternatively, the method may be performed on cells in viva

The invention further provides a method of inhibiting growth and/or metastasis of a tumour in a human patient, the method comprising administering to the patient a therapeutically effective amount of an antibody according to any of the first three aspects of the invention, or a nucleic acid molecule or expression vector encoding the antibody, or a compound as defined in the seventh aspect of the invention. Optionally, the method may further comprise administering at least one additional anti-cancer agent to the patient, as described below.

Terms such as “treating” or “treatment” or “to treat” or “alleviating” or “to alleviate” refer to both therapeutic measures that cure, slow down, lessen symptoms of, and/or halt progression of a diagnosed pathologic condition or disorder and prophylactic or preventative measures that prevent and/or slow the development of a targeted pathologic condition or disorder. Thus those in need of treatment include those already with the disorder; those prone to have the disorder; and those in whom the disorder is to be prevented. In certain embodiments, a subject is successfully “treated” according to the methods of the present invention if the patient shows one or more of the following: a reduction in the number of or complete absence of cancer cells; a reduction in the tumour size; inhibition of or an absence of cancer cell infiltration into peripheral organs including, for example, the spread of cancer into soft tissue and bone; inhibition of or an absence of tumour metastasis; inhibition or an absence of tumour growth; relief of one or more symptoms associated with the specific cancer; reduced morbidity and mortality; improvement in quality of life; reduction in tumorigenicity; reduction in the number or frequency of cancer stem cells; or some combination of effects.

Deregulated MET pathways can be induced by transcriptional up regulation, MET gene amplification, specific genetic alterations, or ligand-dependent autocrine or paracrine mechanisms. The most frequent cause of constitutive MET activation in human tumours is increased protein expression as a consequence of transcriptional upregulation, in the absence of gene amplification. In addition, amplification of the MET gene, with consequent protein overexpression and constitutive kinase activation, has been reported in a number of human primary tumours, including gastric and oesophageal carcinomas, non-small-cell lung (NSCL) carcinomas, and medulloblastomas. Tumours of mesenchymal origin, such as osteosarcomas and rhabdomyosarcomas, often utilise autocrine mechanisms by producing HGF. Elevated HGF levels and overexpression of MET are often associated with poor clinical outcomes that include more aggressive disease, increased tumour metastasis, and shortened patient survival. Further, high levels of HGF and/or MET proteins in tumours confer resistance to chemotherapy and radiotherapy. In addition to abnormal HGF and MET expression, the MET pathway can be activated through genetic alternations such as MET mutations, gene amplification, and gene rearrangement. Missense MET mutations are found in all individuals with well-characterised hereditary papillary renal cell carcinomas (PRCC) and in a small subset (13%) of sporadic PRCC samples. Some of the mutations possess oncogenic potential due to increased kinase activity. Trisomy of chromosome 7, where both the HGF and MET genes reside, occurs frequently in PRCC, and results in non-random duplication of the mutant MET allele. In addition, somatic MET mutations have been identified in other human cancers, including gastric, head and neck, liver, ovarian, non-small cell lung and thyroid cancers, as well as in metastases of some of these cancers. Unlike PRCC, where mutations are typically confined to the kinase domain, these mutations are often located in other regions of the receptor, for example, the juxtamembrane domain. In addition to mutation, the MET gene is often amplified in breast, liver, brain, colorectal, gastric, lung and stomach cancers, which is correlated to disease progression in some patients.

Aberrant HGF/MET signalling has been documented in a wide range of human malignancies, including bladder, breast, cervical, colorectal, endometrial, oesophageal, gastric, head and neck, kidney, liver, lung, nasopharyngeal, ovarian, pancreatic, prostate and thyroid cancers, as well as cholangiocarcinoma, osteosarcoma, rhabdomyosarcoma, synovial sarcoma, Kaposi's sarcoma, leiomyosarcomas, and MFH/fibrosarcoma. In addition, abnormal HGF and/or MET expression has also been reported in haematological malignancies such as acute myelogenous leukaemia, adult T-cell leukaemia, chronic myeloid leukaemia, lymphomas and multiple myeloma, as well as other tumours such as melanoma, mesothelioma, Wilms tumour, glioblastomata, and astrocytomas (summarised in Liu et al (2008) Expert Opin. Investig. Drugs 17(7): 997-1011). Preferably, the MET antibodies of the present invention can inhibit both HGF-dependent and HGF-independent tumours.

Accordingly, a further aspect of the invention provides a method of treating cancer in a human patient, the method comprising administering to the patient a therapeutically effective amount of an antibody according to any of the first three aspects of the invention, or a nucleic acid molecule or expression vector encoding the antibody, or a compound according to the seventh aspect of the invention. Optionally, the method may further comprise administering at least one additional anti-cancer agent to the patient, as described below.

The invention thus includes an antibody according to any of the first three aspects of the invention, or a nucleic acid molecule or expression vector encoding the antibody, or a compound according to the seventh aspect of the invention, and, optionally, at least one additional anti-cancer agent, for use in inhibiting activity of human MET on a cell, for use in inhibiting human MET-mediated cellular proliferation and/or migration, for use inhibiting growth of a tumour in a human patient, for use in inhibiting metastasis of a tumour in a human patient, or for use in treating cancer in a human patient, as discussed above.

The invention also includes the use of an antibody according to any of the first three aspects of the invention, or a nucleic acid molecule or expression vector encoding the antibody, or a compound according to the seventh aspect of the invention, and, optionally, at least one additional anti-cancer agent, in the preparation of a medicament for use in inhibiting activity of human MET on a cell, for use in inhibiting human MET-mediated cellular proliferation and/or migration, for use inhibiting growth of a tumour in a human patient, for use in inhibiting metastasis of a tumour in a human patient, or for use in treating cancer in a human patient, as discussed above.

It is appreciated that the antibody or compound of the invention will usually be administered separately from the at least one additional anti-cancer agent. In such an embodiment, the antibody or compound and the additional anti-cancer agent may be administered sequentially, or they may be administered substantially simultaneously, typically through distinct routes of administration.

However, it is appreciated that in some cases, a combined formulation could be useful, for example, if two antibodies were to be administered to the patient and a single infusion would be quicker and easier to administer. Thus, in certain embodiments, the pharmaceutical composition may further comprise the at least one additional anti-cancer agent.

The additional anticancer agent may be selected from alkylating agents including nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulphan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); natural products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes; miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; cell cycle inhibitors; proteosome inhibitors such as Bortezomib (Velcade); signal transductase (e.g. tyrosine kinase) inhibitors such as Imatinib (Glivec®), COX-2 inhibitors, and hormone agonists/antagonists such as flutamide and tamoxifen.

The clinically used anticancer agents are typically grouped by mechanism of action: Alkylating agents, Topoisomerase I inhibitors, Topoisomerase II inhibitors, RNA/DNA antimetabolites, DNA antimetabolites and Antimitotic agents. The US NIH/National Cancer Institute website lists 122 compounds (http://dtp.nci.nih.gov/docs/cancer/searches/standard_mechanism.html), all of which may be used in conjunction with the compound. They include Alkylating agents including Asaley, AZQ, BCNU, Busulfan, carboxyphthalatoplatinum, CBDCA, CCNU, CHIP, chlorambucil, chlorozotocin, cis-platinum, clomesone, cyanomorpholino-doxorubicin, cyclodisone, dianhydrogalactitol, fluorodopan, hepsulfam, hycanthone, melphalan, methyl CCNU, mitomycin C, mitozolamide, nitrogen mustard, PCNU, piperazine, piperazinedione, pipobroman, porfiromycin, spirohydantoin mustard, teroxirone, tetraplatin, thio-tepa, triethylenemelamine, uracil nitrogen mustard, Yoshi-864; anitmitotic agents including allocolchicine, Halichondrin B, colchicine, a colchicine derivative, dolastatin 10, maytansine, rhizoxin, taxol, taxol derivative, thiocolchicine, trityl cysteine, vinblastine sulphate, vincristine sulphate; Topoisomerase I Inhibitors including camptothecin, camptothecin, Na salt, aminocamptothecin, 20 camptothecin derivatives, morpholinodoxorubicin; Topoisomerase II Inhibitors including doxorubicin, amonafide, m-AMSA, anthrapyrazole derivative, pyrazoloacridine, bisantrene HCL, daunorubicin, deoxydoxorubicin, mitoxantrone, menogaril, N,N-dibenzyl daunomycin, oxanthrazole, rubidazone, VM-26, VP-16; RNA/DNA antimetabolites including L-alanosine, 5-azacytidine, 5-fluorouracil, acivicin, 3 aminopterin derivatives, an antifol, Baker's soluble antifol, dichlorallyl lawsone, brequinar, ftorafur (pro-drug), 5,6-dihydro-5-azacytidine, methotrexate, methotrexate derivative, N-(phosphonoacetyl)-L-aspartate (PALA), pyrazofurin, trimetrexate; DNA antimetabolites including, 3-HP, 2′-deoxy-5-fluorouridine, 5-HP, alpha-TGDR, aphidicolin glycinate, ara-C, 5-aza-2′-deoxycytidine, beta-TGDR, cyclocytidine, guanazole, hydroxyurea, inosine glycodialdehyde, macbecin II, pyrazoloimidazole, thioguanine and thiopurine.

It may be preferred that the at least one additional anticancer agent is selected from cisplatin, carboplatin, 5-fluorouracil, paclitaxel, mitomycin C, doxorubicin, gemcitabine, tomudex, pemetrexed, methotrexate, irinotecan, oxaliplatin, or combinations thereof.

It may also be preferred that the further anticancer agent is a platinum-based chemotherapeutic agent. Clinically approved platinum-based chemotherapeutic agents include carboplatin (cis-diammine(cyclobutane-1,1-dicarboxylate-O,O′)platinum(II); CAS Registry Number 41575-94-4), cisplatin ((SP-4-2)-diamminedichloridoplatinum; CAS Registry number 15663-271), and oxaliplatin (R1R,2R)-cyclohexane-1,2-diamineliethanedioato-O,O′)platinum(II); CAS Registry Number 63121-00-6). Oxaliplatin may also be typically administered with fluorouracil and leucovorin in a combination known as FOLFOX. These platinum-based chemotherapeutic agents are typically administered intravenously as a short-term infusion in physiological saline, as is well known in the art.

It is appreciated that since MET upregulation is a known mechanism of resistance to EGFR inhibitors (e.g., Engelman et al., 2007, “MET amplification leads to gefitinib resistance in lung cancer by activating ERBB3 signaling”. Science 316:1039-43), in a preferred embodiment, the additional anti-cancer agent may be an EGFR inhibitor, such as erlotinib or gefitinib, or an anti-EGFR antibody such as Erbitux.

In certain specific embodiments, the treatment may comprise administration of the anti-MET antibody, an anti-EGFR agent and a chemotherapy drug.

Clinical trials with anti-HGF antibodies, and MET inhibitors including anti-MET antibodies, have been conducted using additional anticancer agents including bevacizumab, capecitabine, epirubicin and cisplatin, panitumumab, mitoxantrone and prednisone, cisplatin and pemetrexed, cisplatin and etoposide, carboplatin and etoposide, gefitinib, bevacizumab and paclitaxel, erlotinib, cetuximab and irinotecan, gemcitabine, sorafenib, lapatinib, docetaxel, topotecan, paclitaxel and carboplatin, ketonazole, the pan-HER inhibitor PF-00299804, rifampin, pemetrexed, rosiglitazone, and temozolomide with radiation therapy. Accordingly, in other embodiments, it may be preferred that the additional anti-cancer agent is one of these agents or combination of agents.

When the additional anticancer agent or combination of agents has been shown to be particularly effective for a specific tumour type, it may be preferred that the antibody or compound of the invention is used in combination with that further anticancer agent(s) to treat that specific tumour type.

The invention may be useful in treating any of those tumours listed above in which the MET gene amplification, MET gene mutation, aberrant HGF/MET signalling, and abnormal HGF and/or MET expression have been reported. Thus, the methods, uses antibodies and compounds of the invention can be used to treat bladder cancer, brain cancer, breast cancer, cervical cancer, colon cancer, colorectal cancer, endometrial cancer, gastric cancer, head and neck cancer, kidney cancer, liver cancer, lung cancer including small cell and non-small-cell lung cancer, medulloblastoma, nasopharyngeal cancer, oesophageal cancer, osteosarcoma, ovarian cancer, pancreatic cancer, papillary renal cell carcinoma, prostate cancer, rhabdomyosarcoma, stomach cancer, thyroid cancer, cholangiocarcinoma, osteosarcoma, rhabdomyosarcoma, synovial sarcoma, Kaposi's sarcoma, leiomyosarcomas, fibrosarcoma, leukaemia including acute myelogenous leukaemia, adult T-cell leukaemia and chronic myeloid leukaemia, lymphoma, multiple myeloma, melanoma, mesothelioma, Wilms tumour, glioma, glioblastomata and astrocytoma.

In a more specific embodiment, the tumour or cancer to be treated may be breast cancer, pancreatic cancer, colon cancer, gastric cancer or lung cancer.

It is appreciated that MET is often mutated in cancer cells, and it is preferred that the antibody for treating cancer should also bind to MET as present on the cancer cells (i.e., the mutated versions of MET). A MET inhibitor antibody could be tested against cell lines engineered to express MET proteins containing activating mutations (e.g., Bellon et al, 2008). In addition, the antibody can be tested against ligand-independent cell lines which have constitutive MET pathway activation as a result of high level amplification of MET such as MKN45 cells (Smolen et al, 2006).

Many suitable formulations and routes of administration for antibodies and compounds of the invention are described above. It is appreciated, however, that in certain embodiments the therapeutic agent may be a nucleic acid molecule, e.g., an expression vector, encoding the antibody or compound (when the further moiety on the compound is a polypeptide). Polynucleotides may be administered by any effective method, for example, parenterally (e.g. intravenously, subcutaneously, intramuscularly) or by oral, nasal or other means which permit the polynucleotides to access and circulate in the patient's bloodstream. Polynucleotides administered systemically preferably are given in addition to locally administered polynucleotides, but also have utility in the absence of local administration. A dosage in the range of from about 0.1 to about 10 grams per administration to an adult human generally will be effective for this purpose.

The polynucleotide may be administered as a suitable genetic construct as is described below and delivered to the patient where it is expressed. Typically, the polynucleotide in the genetic construct is operatively linked to a promoter which can express the compound in the cell. The genetic constructs of the invention can be prepared using methods well known in the art, for example in Sambrook et al (2001).

Although genetic constructs for delivery of polynucleotides can be DNA or RNA, it is preferred if they are DNA.

Preferably, the genetic construct is adapted for delivery to a human cell. Means and methods of introducing a genetic construct into a human cell are known in the art. For example, the constructs of the invention may be introduced into cells by any convenient method, for example methods involving retroviruses, so that the construct is inserted into the genome of the cell. For example, in Kuriyama et al (1991, Cell Struc. and Func. 16, 503-510) purified retroviruses are administered. Retroviral DNA constructs comprising a polynucleotide as described above may be made using methods well known in the art. To produce active retrovirus from such a construct it is usual to use an ecotropic psi2 packaging cell line grown in Dulbecco's modified Eagle's medium (DMEM) containing 10% foetal calf serum (FCS). Transfection of the cell line is conveniently by calcium phosphate co-precipitation, and stable transformants are selected by addition of G418 to a final concentration of 1 mg/ml (assuming the retroviral construct contains a neo^{R} gene). Independent colonies are isolated and expanded and the culture supernatant removed, filtered through a 0.45 μm pore-size filter and stored at −70° C. For the introduction of the retrovirus into tumour cells, for example, it is convenient to inject directly retroviral supernatant to which 10 μg/ml Polybrene has been added. For tumours exceeding 10 mm in diameter it is appropriate to inject between 0.1 ml and 1 ml of retroviral supernatant; preferably 0.5 ml.

Alternatively, cells which produce retroviruses may be injected (Culver et al (1992, Science 256, 1550-1552). The retrovirus-producing cells so introduced are engineered to actively produce retroviral vector particles so that continuous productions of the vector occurred within the tumour mass in situ. Targeted retroviruses are also available for use in the invention; for example, sequences conferring specific binding affinities may be engineered into pre-existing viral env genes (see Miller & Vile (1995) Faseb J. 9, 190-199, for a review of this and other targeted vectors for gene therapy).

Other methods involve simple delivery of the construct into the cell for expression therein either for a limited time or, following integration into the genome, for a longer time. An example of the latter approach includes liposomes (Nässander et al (1992) Cancer Res. 52, 646-653).

Other methods of delivery include adenoviruses carrying external DNA via an antibody-polylysine bridge (see Curiel (1993) Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone. The adenovirus, because it contains unaltered fibre and penton proteins, is internalised into the cell and carries into the cell with it the DNA construct of the invention. It is preferred if the polycation is polylysine.

In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulphide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the tumour cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used.

It will be appreciated that “naked DNA” and DNA complexed with cationic and neutral lipids may also be useful in introducing the DNA of the invention into cells of the individual to be treated. Non-viral approaches to gene therapy are described in Ledley (1995, Human Gene Therapy 6, 1129-1144).

Although for cancer/tumours of specific tissues it may be useful to use tissue-specific promoters in the vectors encoding a polynucleotide inhibitor, this is not essential, as the risk of expression of the antibody in the body at locations other than the cancer/tumour would be expected to be tolerable in compared to the therapeutic benefit to a patient suffering from a cancer/tumour.

Targeted delivery systems are also known, such as the modified adenovirus system described in WO 94/10323, wherein, typically, the DNA is carried within the adenovirus, or adenovirus-like, particle. Michael et al (1995) Gene Therapy 2: 660-668, describes modification of adenovirus to add a cell-selective moiety into a fibre protein. Mutant adenoviruses which replicate selectively in p53-deficient human tumour cells, such as those described in Bischoff et al (1996) Science 274: 373-376 are also useful for delivering genetic constructs to a cell. Other suitable viruses, viral vectors or virus-like particles include lentivirus and lentiviral vectors, HSV, adeno-assisted virus (AAV) and MV-based vectors, vaccinia and parvovirus.

Methods of delivering polynucleotides to a patient are well known to a person of skill in the art and include the use of immunoliposomes, viral vectors (including vaccinia, modified vaccinia, adenovirus and adeno-associated viral (AAV) vectors), and by direct delivery of DNA, e.g. using a gene-gun and electroporation. Furthermore, methods of delivering polynucleotides to a target tissue of a patient for treatment are also well known in the art.

Methods of targeting and delivering therapeutic agents directly to specific regions of the body are well known to a person of skill in the art.

For example, U.S. Pat. No. 6,503,242 describes an implanted catheter apparatus for delivering therapeutic agents directly to the hippocampus. Methods of targeting and delivering agents to the brain can be used for the treatment of cancer/tumours of the brain. In one embodiment, therapeutic agents including vectors can be distributed throughout a wide region of the CNS by injection into the cerebrospinal fluid, e.g., by lumbar puncture (See e.g., Kapadia et al (1996) Neurosurg 10: 585-587). Alternatively, precise delivery of the therapeutic agent into specific sites of the brain can be conducted using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and a number of images are used to determine a target site and trajectory for microinjection of the therapeutic agent. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. The therapeutic agent can be delivered to regions of the CNS such as the hippocampus, cells of the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), or combinations, thereof. In another embodiment, the therapeutic agent is delivered using other delivery methods suitable for localised delivery, such as localised permeation of the blood-brain barrier. US patent application no. 2005/0025746 describes delivery systems for localised delivery of an adeno-associated virus vector (AAV) vector encoding a therapeutic agent to a specific region of the brain.

When a therapeutic agent for the treatment of a cancer/tumour of, for example, the brain, is encoded by a polynucleotide, it may be preferable for its expression to be under the control of a suitable tissue-specific promoter. Central nervous system (CNS) specific promoters such as, neuron-specific promoters (e.g., the neurofilament promoter (Byrne and Ruddle (1989) Proc. Natl. Acad. Sci. USA 86: 5473-5477) and glial specific promoters (Morii et al (1991) Biochem. Biophys Res. Commun. 175: 185-191) are preferably used for directing expression of a polynucleotide preferentially in cells of the CNS. Preferably, the promoter is tissue specific and is essentially not active outside the central nervous system, or the activity of the promoter is higher in the central nervous system than in other cells or tissues. For example, the promoter may be specific for the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), or combinations, thereof. The promoter may be specific for particular cell types, such as neurons or glial cells in the CNS. If it is active in glial cells, it may be specific for astrocytes, oligodendrocytes, ependymal cells, Schwann cells, or microglia. If it is active in neurons, it may be specific for particular types of neurons, e.g., motor neurons, sensory neurons, or interneurons. The promoter may be specific for cells in particular regions of the brain, for example, the cortex, stratium, nigra and hippocampus.

Suitable neuronal specific promoters include, but are not limited to, neuron specific enolase (NSE; Olivia et al (1991) Genomics 10: 157-165); GenBank Accession No: X51956), and human neurofilament light chain promoter (NEFL; Rogaev et al (1992) Hum. Mol. Genet. 1: 781); GenBank Accession No: L04147). Glial specific promoters include, but are not limited to, glial fibrillary acidic protein (GFAP) promoter (Morii et al (1991); GenBank Accession No: M65210), S100 promoter (Morii et al (1991); GenBank Accession No: M65210) and glutamine synthase promoter (Van den et al (1991) Biochem. Biophys. Acta. 2: 249-251); GenBank Accession No: X59834). In a preferred embodiment, the gene is flanked upstream (i.e., 5′) by the neuron specific enolase (NSE) promoter. In another preferred embodiment, the gene of interest is flanked upstream (i.e., 5′) by the elongation factor 1 alpha (EF) promoter. A hippocampus specific promoter that might be used is the hippocampus specific glucocorticoid receptor (GR) gene promoter.

Alternatively, for treatment of cancer/tumours of the heart, Svensson et al (1999) describes the delivery of recombinant genes to cardiomyocytes by intramyocardial injection or intracoronary infusion of cardiotropic vectors, such as recombinant adeno-associated virus vectors, resulting in transgene expression in murine cardiomyocytes in vivo (Svensson et al (1999) “Efficient and stable transduction of cardiomyocytes after intramyocardial injection or intracoronary perfusion with recombinant adeno-associated virus vectors.” Circulation. 99: 201-5). Melo et al review gene and cell-based therapies for heart disease Melo et al (2004) “Gene and cell-based therapies for heart disease.”FASEB J. 18(6): 648-63). An alternative preferred route of administration is via a catheter or stent. Stents represent an attractive alternative for localized gene delivery, as they provide a platform for prolonged gene elution and efficient transduction of opposed arterial walls. This gene delivery strategy has the potential to decrease the systemic spread of the viral vectors and hence a reduced host immune response. Both synthetic and naturally occurring stent coatings have shown potential to allow prolonged gene elution with no significant adverse reaction (Sharif et al (2004) “Current status of catheter- and stent-based gene therapy.” Cardiovasc Res. 64(2): 208-16).

It may be desirable to be able to temporally regulate expression of the polynucleotide inhibitor in the cell, although this is not essential for the reasons given above. Thus, it may be desirable that expression of the polynucleotide is directly or indirectly (see below) under the control of a promoter that may be regulated, for example by the concentration of a small molecule that may be administered to the patient when it is desired to activate or, more likely, repress (depending upon whether the small molecule effects activation or repression of the said promoter) expression of the antibody from the polynucleotide. This may be of particular benefit if the expression construct is stable, i.e., capable of expressing the inhibitor (in the presence of any necessary regulatory molecules), in the cell for a period of at least one week, one, two, three, four, five, six, eight months or one or more years. Thus the polynucleotide may be operatively linked to a regulatable promoter. Examples of regulatable promoters include those referred to in the following papers: Rivera et al (1999) Proc Natl Acad Sci USA 96(15), 8657-62 (control by rapamycin, an orally bioavailable drug, using two separate adenovirus or adeno-associated virus (AAV) vectors, one encoding an inducible human growth hormone (hGH) target gene, and the other a bipartite rapamycin-regulated transcription factor); Magari et al (1997) J Clin Invest 100(11), 2865-72 (control by rapamycin); Bueler (1999) Biol Chem 380(6), 613-22 (review of adeno-associated viral vectors); Bohl et al (1998) Blood 92(5), 1512-7 (control by doxycycline in adeno-associated vector); Abruzzese et al (1996) J Mol Med 74(7), 379-92 (review of induction factors, e.g. hormones, growth factors, cytokines, cytostatics, irradiation, heat shock and associated responsive elements).

A further aspect of the invention provides a method of detecting the presence of human MET on a cell, or a method of imaging a cell or tissue expressing human MET, the method comprising contacting the cell or tissue with a compound according to the sixth aspect of the invention, and detecting or imaging the detectable label.

In this aspect, it may be preferred that the antibody is in an scFv format. scFv antibodies are often highly suitable for use as imaging agents as their smaller size and shorter in vivo half-life are often advantageous with respect to obtaining clear images with low background signal.

In a preferred embodiment, the cell is a cancer or tumour cell, for example from one of the cancers/tumours described above.

It is appreciated that the method may be performed on cells or tissue in vitro. Alternatively, the method may be performed on cells or tissue in vivo.

This aspect of the invention includes a method of detecting or imaging a tumour in a human patient, the method comprising administering to the patient an effective amount of a compound according to the sixth aspect of the invention, and detecting or imaging the detectable label, thereby to detect or image the tumour in the human patient.

This aspect of the invention includes a compound as defined in the sixth aspect of the invention for use in detecting or imaging a tumour in a human patient.

Suitable methods for using a compound comprising an anti-MET antibody and a detectable label detecting the presence of human MET on a cell, for imaging a cell or tissue expressing human MET, and for detecting or imaging a tumour in a patient are very well known in the art.

MET inhibitors have also been proposed for use in treating endometriosis (WO 2011/067189). Accordingly, another aspect of the invention provides a method of treating endometriosis in a human patient, the method comprising administering to the patient a therapeutically effective amount of an antibody according to any of the first three aspects of the invention, or a nucleic acid molecule or expression vector encoding the antibody, optionally, in combination with at least one additional treatment for endometriosis.

The invention includes an antibody according to any of the first three aspects of the invention, or a nucleic acid molecule or expression vector encoding the antibody, for use in treating endometriosis in a human patient.

The invention also includes the use of an antibody according to any of the first three aspects of the invention, or a nucleic acid molecule or expression vector encoding the antibody, in the preparation of a medicament for treating endometriosis in a human patient.

MET and HGF have also been shown to be involved in the regulation of glucose metabolism (Fafalios et al, 2011, “A hepatocyte growth factor receptor (Met)-insulin receptor hybrid governs hepatic glucose metabolism”. Nature Medicine 612: 1-9). Accordingly, another aspect of the invention provides a method of treating hypoglycaemia in a human patient, the method comprising administering to the patient a therapeutically effective amount of an antibody according to any of the first three aspects of the invention, optionally, in combination with at least one additional treatment for hypoglycaemia.

The invention includes an antibody according to any of the first three aspects of the invention for use in treating hypoglycaemia in a human patient. The invention also includes the use of an antibody according to any of the first three aspects of the invention, in the preparation of a medicament for treating hypoglycaemia in a human patient.

MET and HGF have also been shown to be involved in the vasculoproliferative phase of inflammatory arthritides, such as rheumatoid arthritis, by inducing HGF-mediated synovial neovascularisation (Koch et al, 1996, “Hepatocyte growth factor. A cytokine mediating endothelial migration in inflammatory arthritis”, Arthritis & Rheumatism 39: 1566-1575). Accordingly, another aspect of the invention provides a method of treating rheumatoid arthritis in a human patient, the method comprising administering to the patient a therapeutically effective amount of an antibody according to any of the first three aspects of the invention, optionally, in combination with at least one additional treatment for rheumatoid arthritis.

The invention includes an antibody according to any of the first three aspects of the invention for use in treating rheumatoid arthritis in a human patient. The invention also includes the use of an antibody according to any of the first three aspects of the invention, in the preparation of a medicament for treating rheumatoid arthritis in a human patient.

It is further appreciated that Met agonists may also be therapeutically useful, e.g. for use in fibrosis, regeneration and hyperglycaemia. Nevertheless, side-effects to these treatments with Met-agonists can occur. Accordingly, another aspect of the invention provides a method of treating, reversing or minimising side-effects associated with the use of a Met-agonist in a human patient, the method comprising administering to a patient a therapeutically effective amount of an antibody according to any of the first three aspects of the invention.

The invention includes an antibody according to any of the first three aspects of the invention for use in treating, reversing or minimising side-effects associated with the use of a Met-agonist in a human patient.

The invention also includes the use of an antibody according to any of the first three aspects of the invention, in the preparation of a medicament for treating, reversing or minimising side-effects associated with the use of a Met-agonist in a human patient.

In a further aspect, the invention provides a kit comprising an antibody or compound or nucleic acid molecule or expression vector or pharmaceutical composition as defined herein. Thus, there may be provided a kit for use in the therapeutic treatment of the conditions defined herein or for detection of MET as described herein.

In an embodiment, the kit may comprise an antibody according to any of the first three aspects of the invention, or a nucleic acid molecule or expression vector encoding the antibody, or a compound according to the seventh aspect of the invention, and at least one additional anti-cancer agent. Preferences for the additional anti-cancer agent are as described above.

The kit may comprise a first container, a label on the container, and a composition contained within the first container, wherein the composition comprises an antibody according to any of the first two of the invention, or a nucleic acid molecule or expression vector encoding the antibody, or a compound according to the seventh aspect of the invention, which is effective for treating cancer, and wherein the label on the first container indicates that the composition can be used for treating cancer; a second container comprising a pharmaceutically-acceptable buffer; and instructions for using the antibody or compound to treat cancer. Optionally, the kit may also comprise at least one additional anti-cancer agent, typically in a separate sterile container, although it may be included within the first or second container.

Accordingly, the invention includes article of manufacture, comprising a sterile container; a pharmaceutical composition contained within the container, wherein the pharmaceutical composition comprises an antibody according to any of the first three aspects of the invention, or a nucleic acid molecule or expression vector encoding the antibody, or a compound according to the seventh aspect of the invention, which is effective for treating cancer; and a label on the container, wherein the label on the container indicates that the composition can be used for treating cancer; and optionally, further comprising instructions for administering the antibody or nucleic acid molecule or expression vector or compound to a human patient. Further optionally, the article of manufacture may also comprise at least one additional anti-cancer agent, typically, although not necessarily, in a separate sterile container.

Alternatively, the kit may comprise a detectable antibody, for example an antigen-binding fragment of a complete antibody as described above, or a compound comprising the antibody and a detectable moiety, suitable for use in diagnosis. Such a diagnostic kit may comprise, in an amount sufficient for at least one assay, the antibody or compound—as a diagnostic agent—in the form of as a separately packaged reagent. Instructions for use of the packaged reagent are also typically included. Such instructions typically include a tangible expression describing reagent concentrations and/or at least one assay method parameter such as the relative amounts of reagent and sample to be mixed, maintenance time periods for reagent/sample admixtures, temperature, buffer conditions and the like. The kit may further comprise suitable reagents for detecting the antibody, or compound, as is known in the art.

The kit may comprise a first container, a label on the container, and a composition contained within the first container, wherein the composition comprises an antibody an antibody according to any of the first two of the invention, or a compound according to the sixth aspect of the invention, which is effective for detecting or purifying human MET, and wherein the label on the first container indicates that the composition can be used for detecting or purifying human MET; a second container comprising a pharmaceutically-acceptable buffer; and instructions for using the antibody or compound to detect or purify human MET. The kit may further comprise a third (or more) container(s) comprising suitable reagents for detecting the antibody, or compound.

Accordingly, the invention includes article of manufacture, comprising a sterile container; a composition contained within the container, wherein the composition comprises an antibody according to any of the first three aspects of the invention, or a compound according to the sixth aspect of the invention, which is effective for detecting or purifying human MET; and a label on the container, wherein the label on the container indicates that the composition can be used for detecting or purifying human MET; and optionally, further comprising instructions for using the antibody or compound to detect or purify human MET.

A further aspect of the invention provides a method of identifying an antibody that may be useful for the detection or treatment of human cancer, the method comprising:
assaying a test antibody for binding to residues 568-741 of human MET (SEQ ID No: 1), wherein the test antibody is as defined in the first aspect of the invention;
wherein a test antibody that binds to residues 568-741 of MET (SEQ ID No: 1) may be an antibody that is useful for the detection or treatment of human cancer, or a lead compound for identifying an antibody that is useful for the detection or treatment of human cancer.

In an embodiment, the assaying step may comprise predicting whether the test antibody binds to residues 568-741 of MET (SEQ ID No: 1) by molecular modelling in silico.

The capability of the test antibody to bind to the specified region of MET may be measured by any method of detecting/measuring a protein/protein interaction or other compound/protein interaction, as discussed further below. Suitable methods include methods such as, for example, yeast two-hybrid interactions, co-purification, ELISA, co-immunoprecipitation and surface plasmon resonance methods. Thus, the candidate compound may be considered capable of binding to the polypeptide or fragment thereof if an interaction may be detected between the candidate compound and the polypeptide or fragment thereof by ELISA, co-immunoprecipitation or surface plasmon resonance methods or by a yeast two-hybrid interaction or copurification method. It is preferred that the interaction can be detected using a surface plasmon resonance method. Surface plasmon resonance methods are well known to those skilled in the art. Techniques are described in, for example, O'Shannessy DJ (1994) “Determination of kinetic rate and equilibrium binding constants for macromolecular interactions: a critique of the surface plasmon resonance literature” Curr Opin Biotechnol. 5(1):65-71; Fivash et al (1998) “BIAcore for macromolecular interaction.” Curr Opin Biotechnol. 9(1):97-101; Malmqvist (1999) “BIACORE: an affinity biosensor system for characterisation of biomolecular interactions.” Biochem Soc Trans. 27(2):335-40.

The capability of a test antibody to bind to the specified region of MET may also be determined via competitive binding assays with antibody 7A2, i.e. an scFv antibody consisting of the sequence of SEQ ID No: 26.

A still further aspect of the invention provides a method of identifying an antibody that may be useful for the detection or treatment of human cancer, the method comprising:
assaying whether a test antibody competes with the NK1 fragment of HGF/SF for binding to human MET (SEQ ID No: 1);
wherein a test antibody that competes with NK1 for binding to MET may be an antibody that is useful for the detection or treatment of human cancer, or a lead compound for identifying an antibody that is useful for the detection or treatment of human cancer.

In a preferred embodiment of this screening method, the test antibody may be as defined in the first aspect of the invention.

It is appreciated that screening assays which are capable of high throughput operation are particularly preferred. Examples may include cell based assays and protein-protein binding assays. An SPA-based (Scintillation Proximity Assay; Amersham International) system may be used. For example, an assay for identifying an antibody capable of modulating the activity of a protein kinase may be performed as follows. Beads comprising scintillant and a substrate polypeptide that may be phosphorylated may be prepared. The beads may be mixed with a sample comprising the protein kinase and ³²P-ATP or ³³P-ATP and with the test antibody. Conveniently this is done in a multi-well (e.g., 96 or 384) format. The plate is then counted using a suitable scintillation counter, using known parameters for ³²P or ³³P SPA assays. Only ³²P or ³³P that is in proximity to the scintillant, i.e., only that bound to the polypeptide, is detected. Variants of such an assay, for example in which the polypeptide is immobilised on the scintillant, may also be used.

Other methods of detecting polypeptide/polypeptide interactions include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescent labelled entities may be measured by measuring the interaction of the fluorescent labels when in close proximity to each other.

In various embodiments, it is preferred that the test antibody is an antigen-binding fragment of an antibody, such as an Fv fragment (e.g. single chain Fv and disulphide-bonded Fv); a Fab-like fragment (e.g. Fab fragments, Fab′ fragments and F(ab)₂ fragments); or a domain antibody, as described above.

It is appreciated that the identification of a candidate antibody that binds to the specified region of MET may only be an initial step in the drug screening pathway, and the identified antibody may be further selected e.g. for the ability to inhibit MET activity. Thus, in an embodiment, the method further comprises the step of determining whether the test antibody reduces MET mediated cell signalling, for example as indicated by protein phosphorylation, or the step of determining whether the test agent reduces human HGF-dependent cellular proliferation or migration. Suitable methods are very well known in the art, and are described herein and employed in the Examples.

These screening methods may also include the step of determining whether or not the antibody binds to MET as present on cancer cells (e.g., a mutated versions of MET). For example, a MET-antagonist antibody could be tested against cell lines engineered to express MET proteins containing activating mutations (e.g., Bellon et al, 2008). In addition, or alternatively, the antibody can be tested against ligand-independent cancer cell lines which have constitutive MET pathway activation as a result of high level amplification of MET, such as MKN45 cells (Smolen et al, 2006).

It is appreciated that these methods may be a drug screening methods, a term well known to those skilled in the art, and the candidate antibody may be or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of molecular biology or biochemistry, and is preferably a small molecule, and which may be water-soluble. A drug-like compound may additionally exhibit features of selective interaction with a particular protein (i.e., MET) and be bioavailable and/or able to penetrate target cellular membranes or the blood:brain barrier if required, but it will be appreciated that these features are not essential.

The term “lead compound” is similarly well known to those skilled in the art, and may include the meaning that the antibody, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics. Thus, the antibodies may also be subjected to other tests, for example toxicology or metabolism tests, as is well known to those skilled in the art.

In an embodiment, the identified antibody is modified, and the modified antibody is retested. In a related embodiment, an agent having or expected to have similar properties to an antibody identified as a result of the method is retested. Many methods for varying and testing antibody sequences are known in the art, some of which are described herein and have been employed in the Examples.

It is appreciated that the screening methods can be used to identify agents that may be useful in detecting or treating tumours/cancers. Thus an agent identified as a result of the method may be tested for efficacy in a cell model and/or an animal model of cancer. The cellular model of cancer may be an in vitro anti-cancer assay such as a cellular proliferation assay of primary cancer cells or cancer cell lines in vitro, as is well known in the art. The non-human animal models of cancer may be, for example, a model of ovarian, lung, breast, colorectal, pancreatic, gastric, oesophageal, renal, biliary, hepatic, cervical, uterine or prostate cancer. The animal model of cancer may be a xenograft model of cancer. Typically, the animal model of cancer is a mouse model of cancer, which may be nude mouse, SCID mouse or oncomouse model of cancer. Suitable animal models of cancer are known in the art and include Lewis lung carcinoma subcutaneous implants in mice (homograft in Black 57 mice) or HT29 xenografts subcutaneous implants in nude mice. Preferably, the U87MG xenograft in nude mice may be employed as a model.

In a further embodiment, an agent identified as a result of the method is further tested for efficacy and safety in a clinical trial for treatment of cancer, optionally in combination with a further anti-cancer agent (for example as discussed above). Typically, in the context of a clinical trial, the individuals in the trial are human patients with cancer or matched controls.

The invention may comprise the further step of synthesising and/or purifying the identified antibody or the modified antibody. The invention may further comprise the step of formulating the antibody into a pharmaceutically acceptable composition, such as described above. The antibody identified, synthesised and/or purified as a result of the method may be packaged and presented for use in treating cancer.

Thus the invention includes a method for preparing an antibody that may be useful in the treatment of a cancer/tumour, the method comprising identifying an antibody using the screening methods described above and synthesising, purifying and/or formulating the identified antibody. The invention also includes a method of making a pharmaceutical composition comprising the step of mixing the antibody identified using the methods described above with a pharmaceutically acceptable carrier, diluent or excipient.

As used herein, the singular forms “a”, “and”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

The listing or discussion in this specification of an apparently prior-published document should not necessarily be taken as an acknowledgement that the document is part of the state of the art or is common general knowledge. Any document referred to herein is hereby incorporated by reference.

The invention will now be described with the aid of the following Figures and Examples.

FIG. 1: The multidomain structure of MET and HGF. (a) MET is synthesised as a single chain precursor and cleaved by furin during transit through the endoplasmic reticulum yielding a small, N-terminal α-chain and a larger β-chain. The MET ectodomain consists of a large N-terminal Sema domain, which adopts a 7-bladed β-propeller fold and has an irregular cylindrical shape and a stalk structure consisting of four immunoglobulin-like domains. The Sema domain and the stalk structure are separated by a small cysteine-rich (CR) domain. The transmembrane (TM), the long juxtamembrane (JM) sequence, the kinase (K) domain and a C-terminal sequence that contains essential motifs for downstream signalling are also shown. HGF/SF is composed of six domains: an N-terminal (N) domain, 4 copies of the kringle domain (K1-4) and a C-terminal domain (SPH) structurally related to the catalytic domain of serine proteases but enzymatically inactive. HGF/SF contains two MET-binding sites: one in the NK1 fragment and one in the SPH domain. (b) Crystal structure of a SPH-MET complex: the SPH domain of HGF/SF binds to an area of the Sema within the MET α-chain (PDB accession 1SHY). (c) Crystal structure of an InIB-MET complex. InIB binds primarily to the first IG domain of the MET stalk. Structures were drawn with PYMOL.

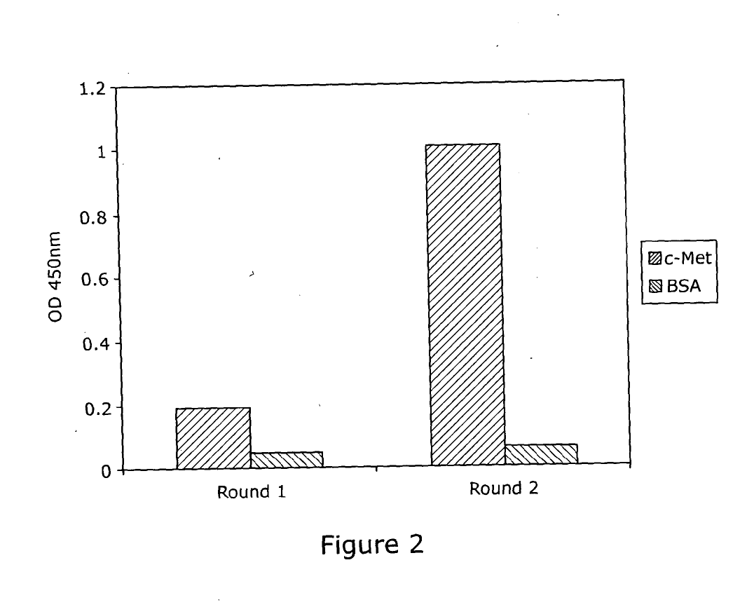

FIG. 2: ELISA using polyclonal phage antibodies selected on c-Met shows binding to c-Met in ELISA.

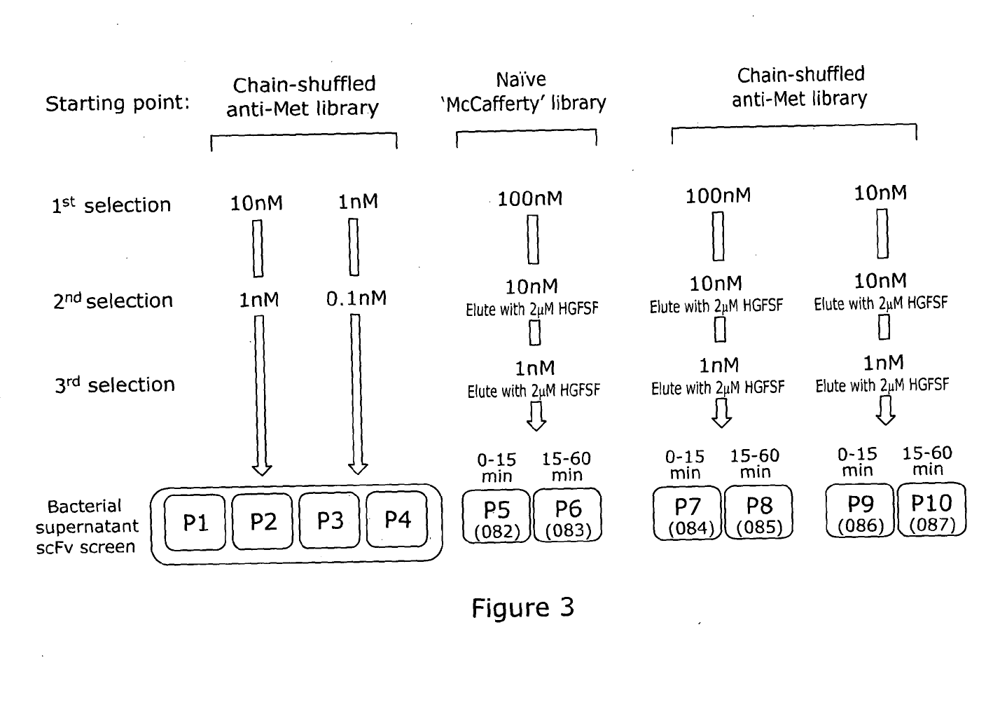

FIG. 3: Outline of selections performed using biotinylated Met-928 as antigen. Where bound phage were eluted with HGF/SF rather than trypsin, this is noted on the Figure. Round 3 elutions were divided into two, with phage eluted in the first 0-15 minutes treated separately from phage eluted in the subsequent 15-45 minutes. P1-P10 indicate the ten 96-well plates into which single clones from the selected populations were picked for expression and storage. Numbers in brackets (i.e. 082-087) indicate the selection code for certain cascades.

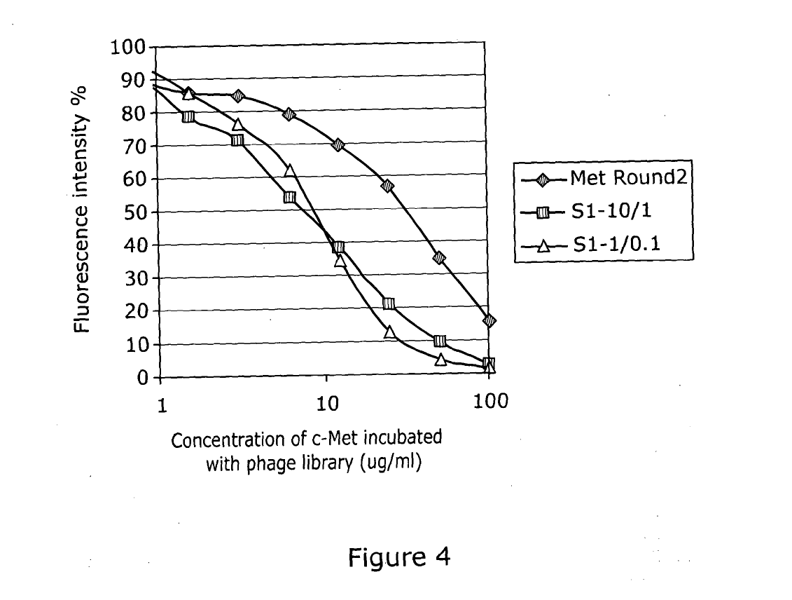

FIG. 4: Polyclonal phage ELISA shows the improved affinity of the affinity-matured populations. Populations arising from 2 rounds of selection on the chain shuffled library “(S1-10/1, S1-10/0.1”) were compared to the original non-shuffled population (“met round 2”). The phage populations were incubated with a range of soluble Met concentrations and the available free antibody was determined by ELISA. With higher affinity populations (i.e. S1-10/1 and 51-10/0.1) lower amounts of soluble Met are required to inhibit binding.

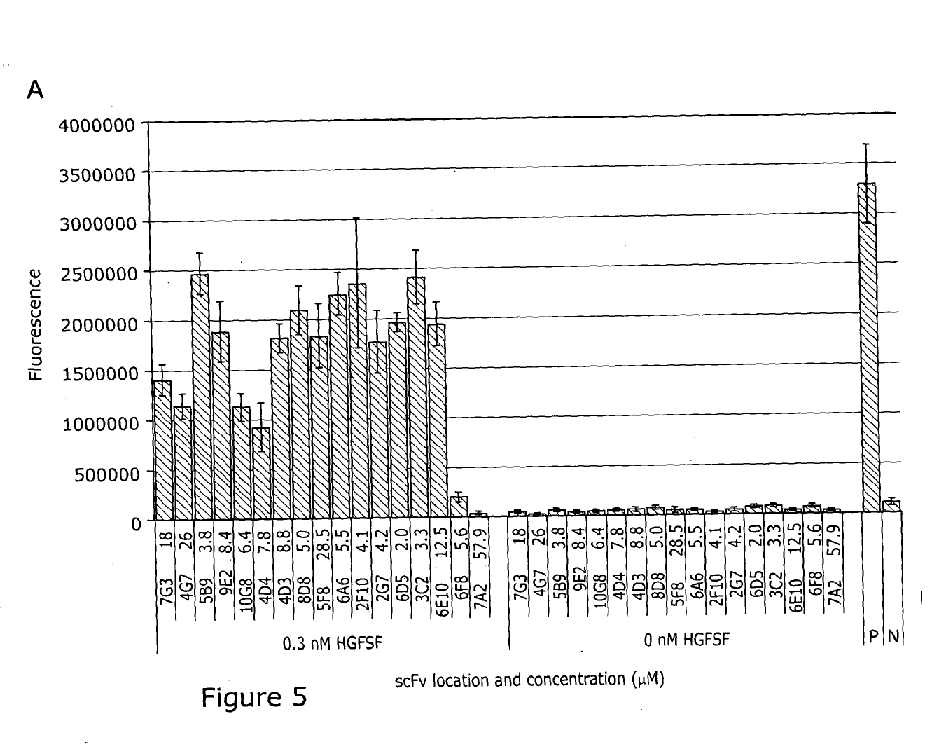

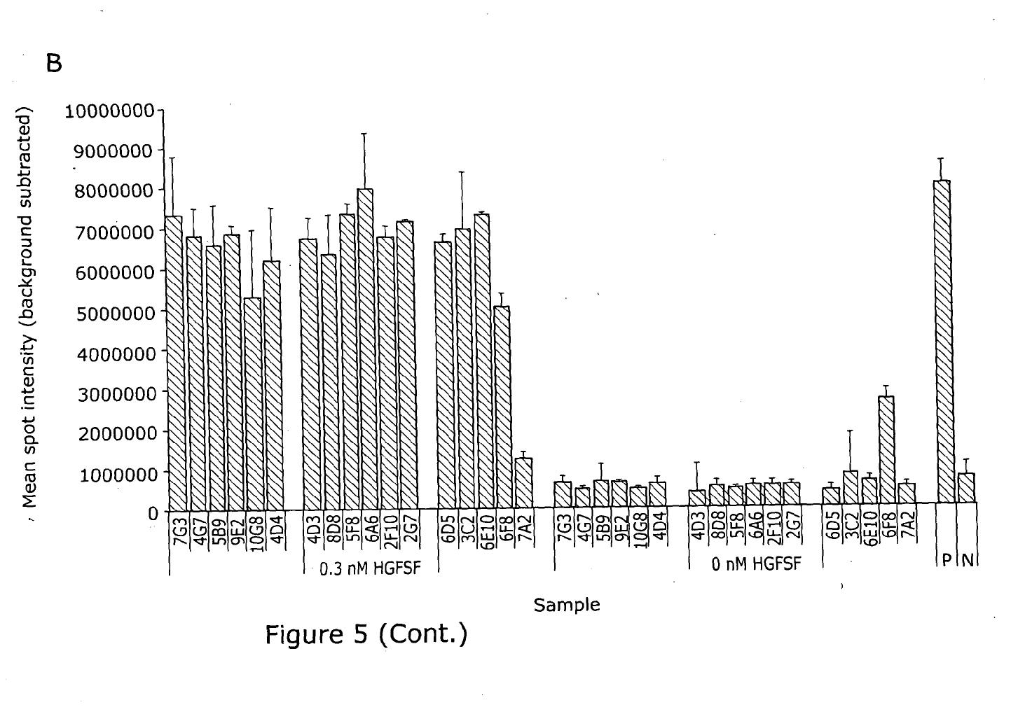

FIG. 5: Migration of SKOV-3 cells towards 300 pM HGF/SF in the presence of scFv at the highest feasible concentration. Screening was performed at these high concentrations in order to identify scFv of all affinities that bind to epitopes with the potential for full HGF/SF antagonism. Data represent mean±range of duplicate wells. P: mean migration in the absence of scFv. N: mean migration in the absence of HGF/SF. P & N: error bars represent standard deviation of a total of 12 wells each. The experiments in part A were conducted at varying concentrations. In part B, all antibodies were tested at approximately 1 μM which provides a side-by-side comparison of activity.

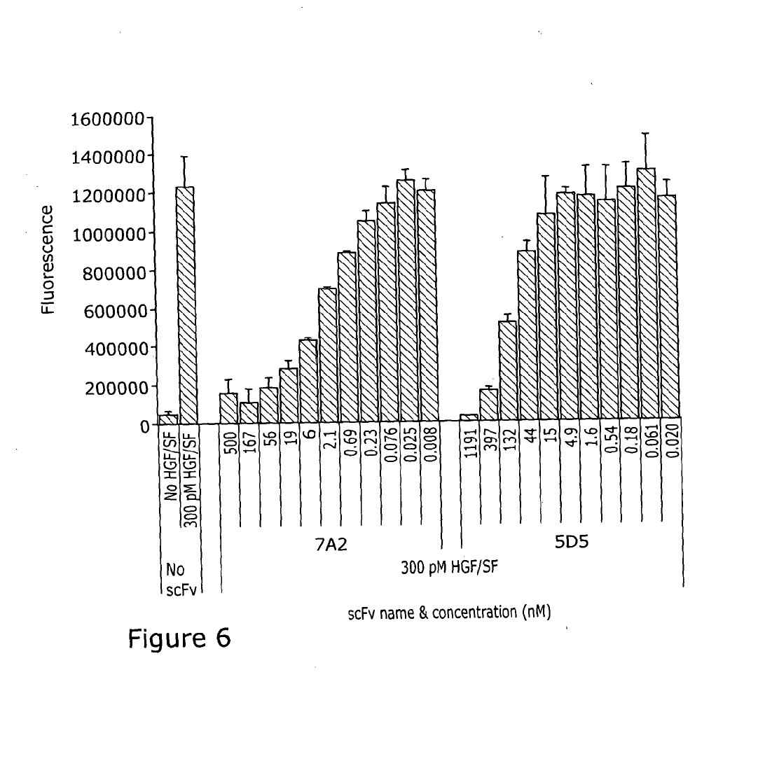

FIG. 6: Migration of SKOV-3 cells towards 300 pM HGF/SF in the presence of multiple concentrations of 7A2 and 5D5 scFv. In this Figure (as in FIGS. 2-5) the antibodies were in scFv format, expressed in E. coli, with 6×His/tri-Flag tags. Data represent mean±range of duplicate wells, or mean±standard deviation of four wells for migration towards HGF/SF in the absence of scFv. The absorbance at 280 nm of purified scFv was multiplied by 0.8 to obtain an estimate of the scFv concentration in units of mg/ml.

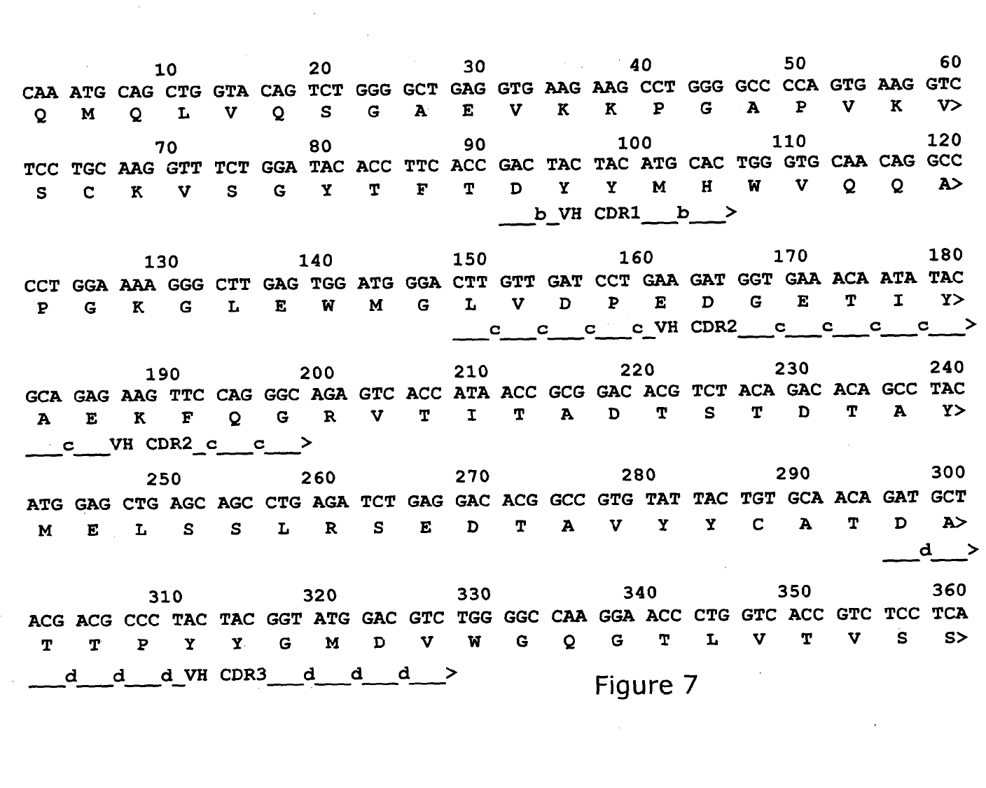

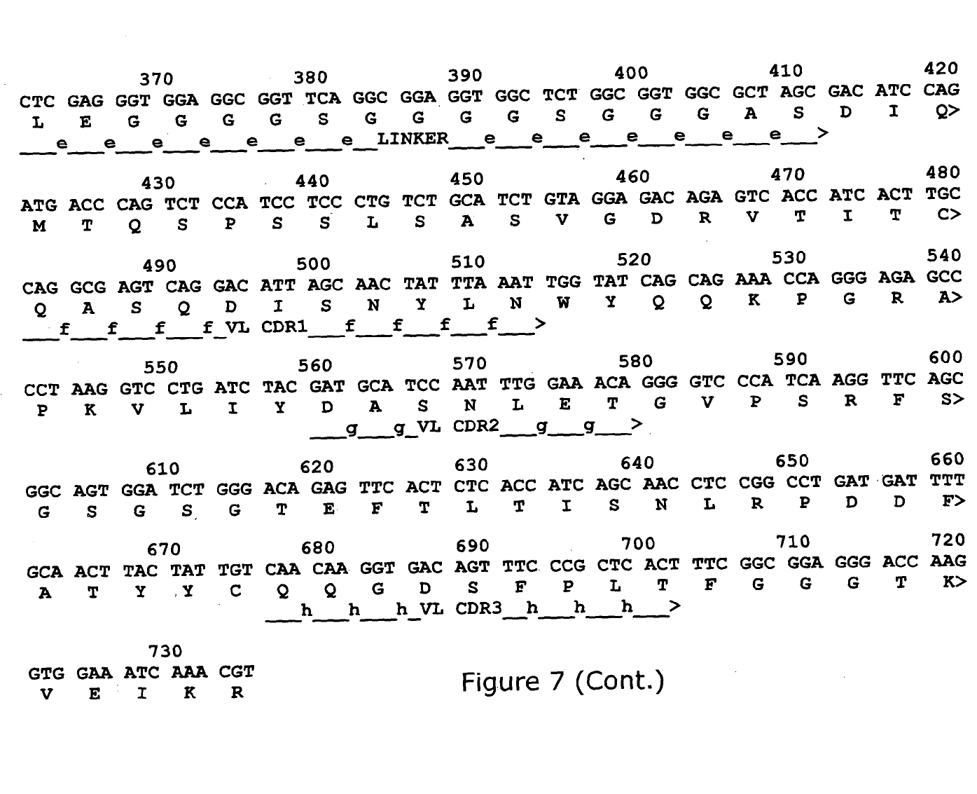

FIG. 7: DNA sequence of anti-Met antibody clone 7A2 showing the encoded amino acid sequence, and the position of the linker and CDR sequences. The VL region is encoded by nucleotides 1-360, followed by the linker, and the VH region is encoded by nucleotides 412-735

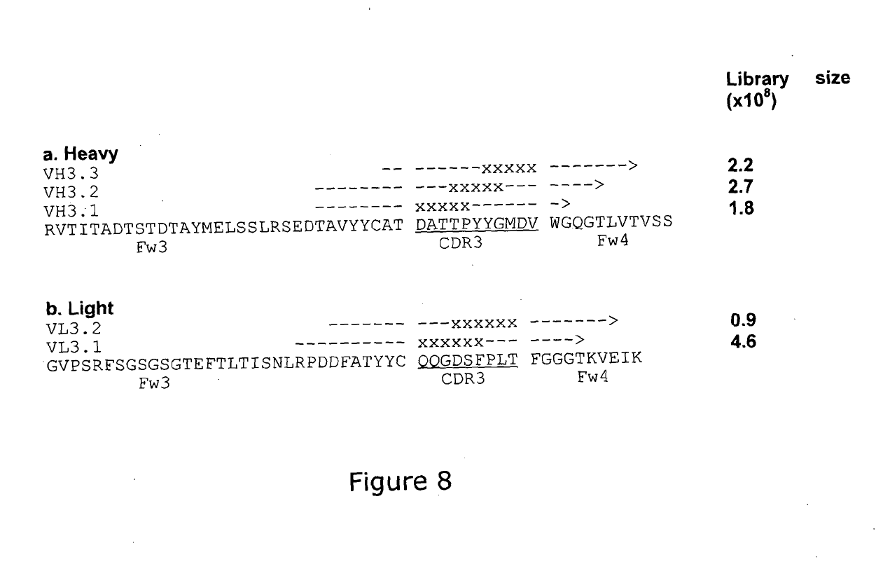

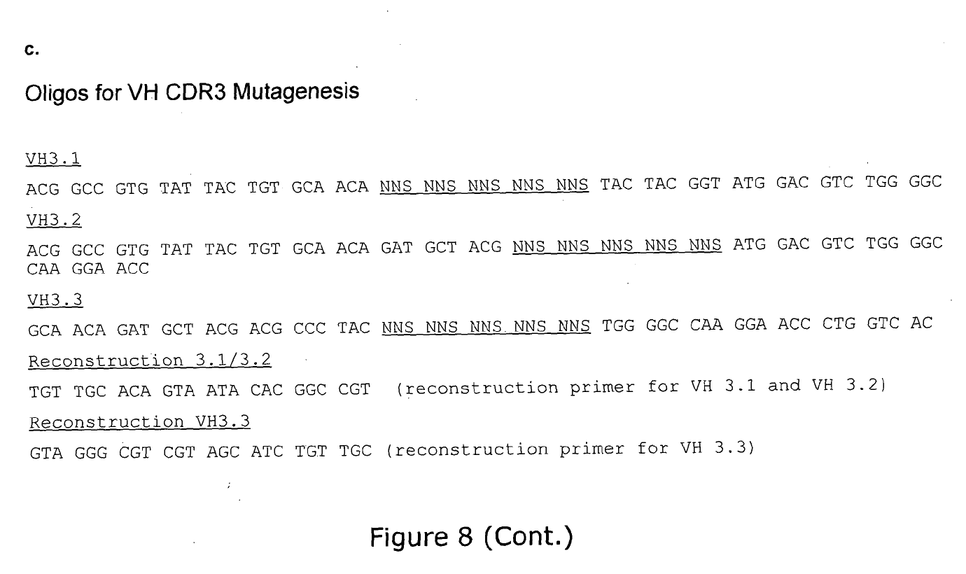

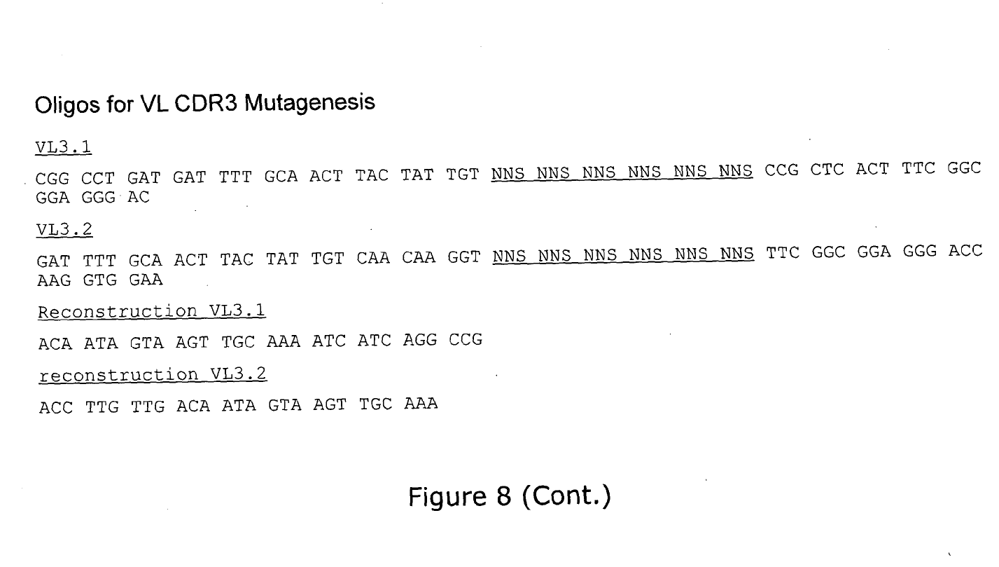

FIG. 8: Randomisation of 7A2 CDR3 regions. The polypeptide sequence of CDR3 of heavy chain (a) and light chain (b) of 7A2 is shown. The location of each of the 5 mutagenic oligos is shown. Dashed lines above amino acids indicate that the original amino acid is encoded in the oligonucleotide. An ‘x’ indicates that the existing codon was replaced with NNS (where N is any nucleotide and S is C or G). Section c provides the DNA sequence of oligonucleotides, described above, which were used to construct libraries. The primers with “reconstruction” in the name are in the reverse orientation and are homologous with the mutagenic primers in the region encoding Framework region 3. These are used to generate a second PCR product with homology to the first product from the mutagenic oligonucleotides, which allow assembly of a complete antibody gene that is randomised in the CDR3 region.

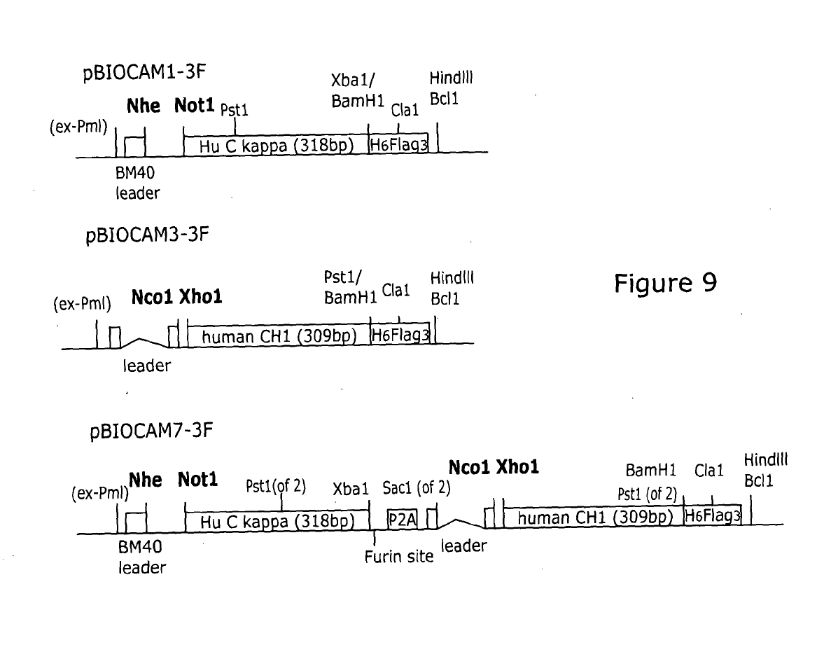

FIG. 9: Construction of pBIOCAM vectors for Fab expression in mammalian cells. The VHs and VLs from all 10 lead antibodies were cloned into the appropriate sites of pBIOCAM7-3F. Transient transfection of human embryonic kidney cells (HEK293 cells) was conducted using standard methods (Tom et al, 2007; Backiwal et al, 2008; and Wulhfard et al, 2010). Following transfection, the HEK293 cells were grown in Gibco Freestyle media with 10% serum added for five days before harvesting the expressed Fab protein. The Fab protein was purified using IMAC on Ni-NTA resin (QIAGEN).

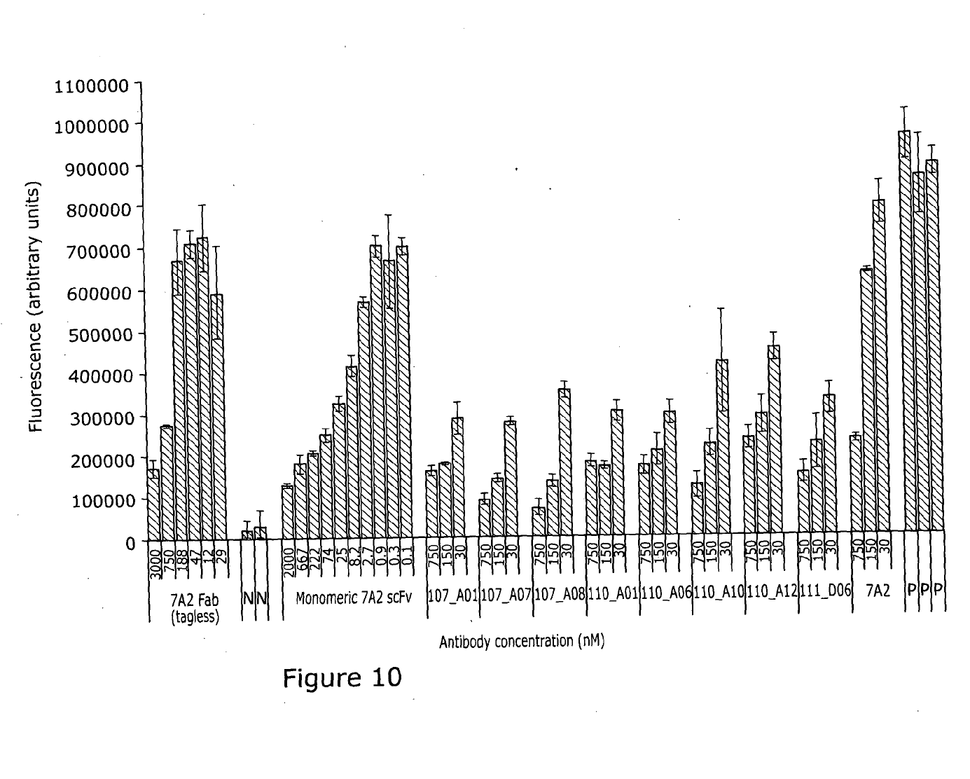

FIG. 10: Migration of SKOV-3 human ovarian carcinoma cells towards 30 pM HGF/SF in the presence of scFv and Fabs. Migration of fluorescently-labelled cells is indicated by the presence of residual fluorescence after removal of non-migrated cells from the upper surface. Tagless 7A2, has no tag, and is in the “hinged” format, i.e., it contains an additional two amino acids (Leu-Glu) inserted at the C terminus of the heavy chain variable region. Monomeric 7A2 scFv is produced in P. pastoris with a His tag only. All other Fabs are in the “hinged” format with an additional Leu-Glu inserted at the C terminus of the heavy chain variable region, and also contain the TriFLAG-His tags. Data represent mean±range of duplicate wells. N: No HGF/SF. P: No antibody.

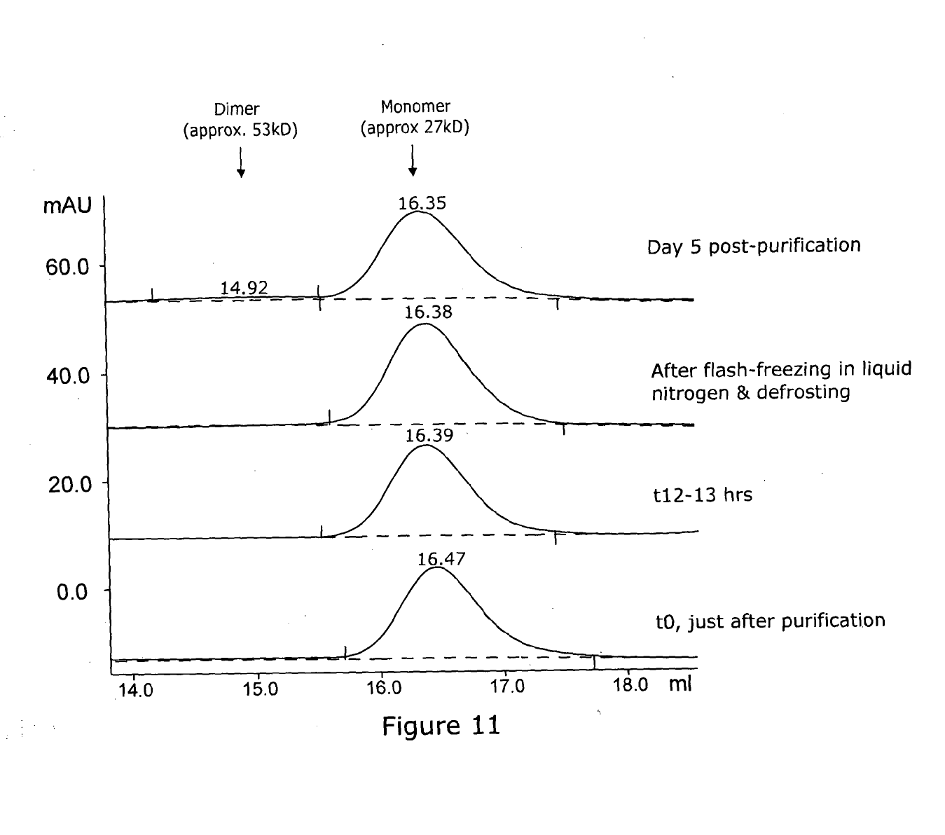

FIG. 11: 7A2 scFv was expressed in P. pastoris and monomeric scFv was purified. The purified monomeric scFv was stored at 4° C. in 25 mM Tris pH8.0, 500 mM NaCl and remains monomeric for at least 5 days post-isolation. FIG. 11 shows 4 μM 7A2 scFv analysed on a Superdex 200 10/300 size exclusion column at several timepoints post-isolation.

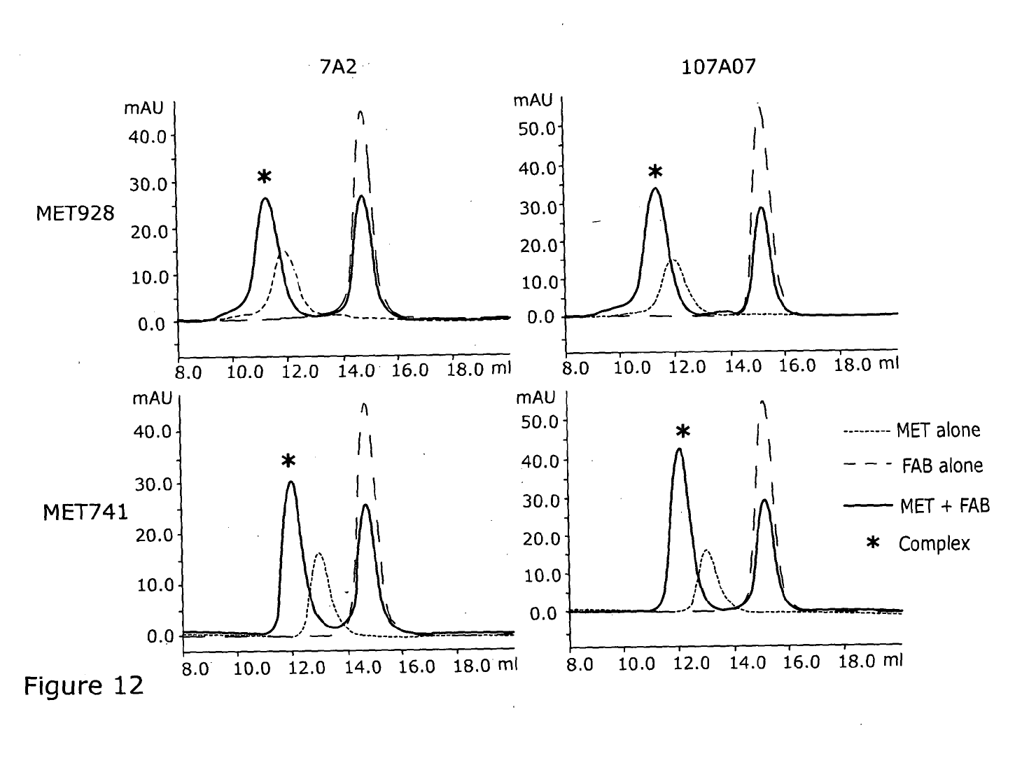

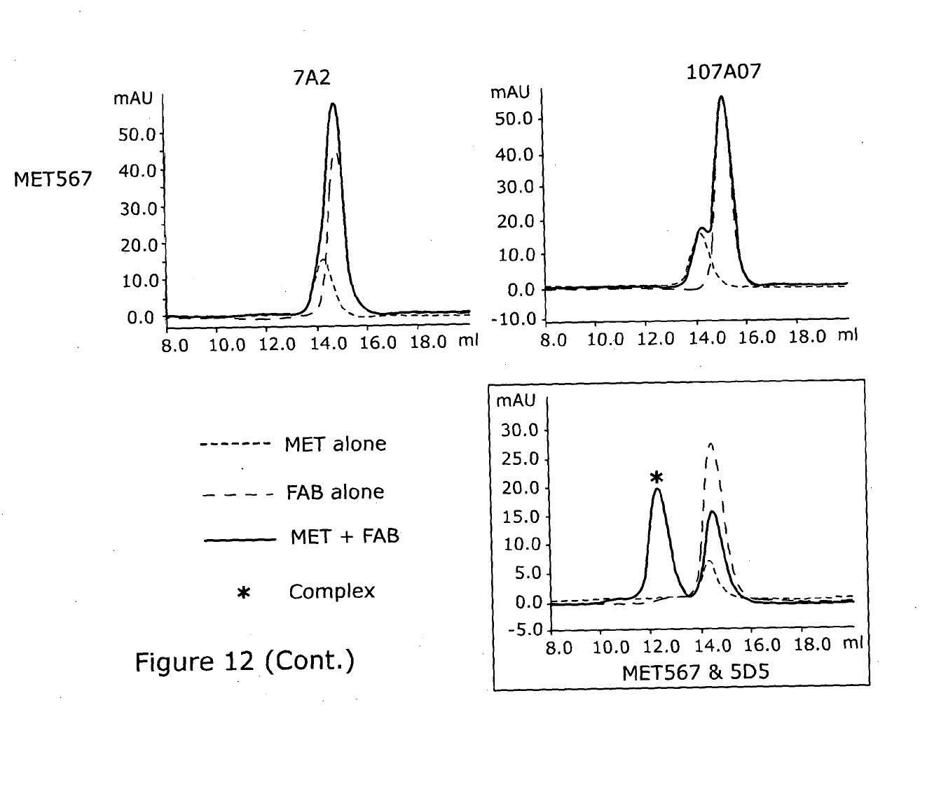

FIG. 12: 4 μM MET928 (upper two panels), MET741 (middle two panels) or MET567 (lower two panels) were incubated with 6 μM of either 7A2 Fab (left panels) or 107_A07 Fab (right panels) for 140 minutes at room temperature before centrifugation and loading onto a Superdex 200 10/300 size exclusion chromatography column. 7A2 is tagless and “hinged”, and 107A07 is tagless and “native” (i.e. not hinged).

The vertical axis indicates absorbance at 280 nm; the horizontal axis indicates the elution volume. Boxed insert: 2 μM MET567 was incubated with 3 μM 5D5 Fab under identical conditions and analysed as described above.

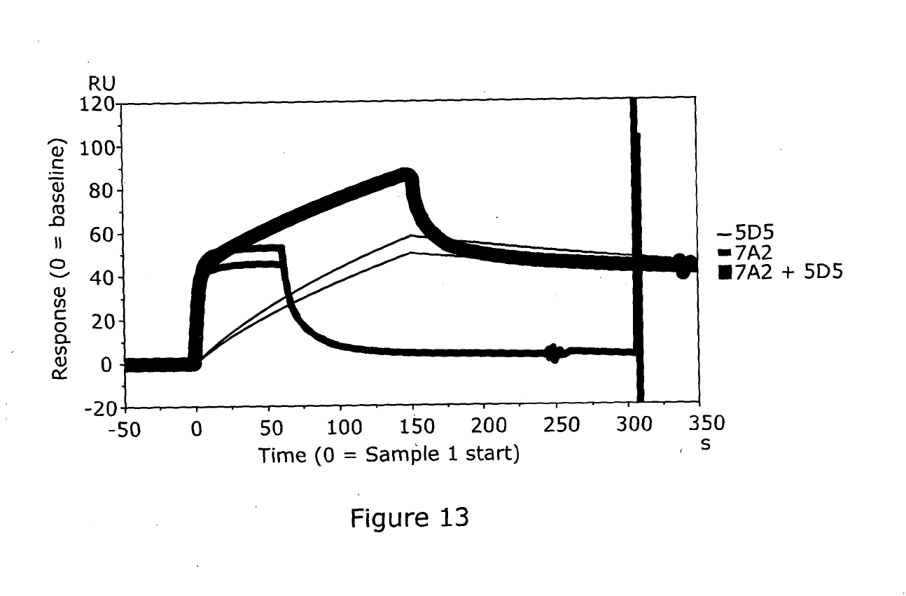

FIG. 13: A CM5 Biacore chip coated with MET928 was exposed to 125 nM 5D5 Fab (lower thin line), 250 nM 5D5 Fab (upper thin line), 134 nM 7A2 scFv (lower medium thickness line), 268 nM 7A2 scFv (upper medium thickness line), or a mixture containing 134 nM 7A2 scFv and 125 nM 5D5 Fab (heavy line). Sensorgrams are shown overlapping and aligned to the start of each injection. Injection times were 60 seconds (7A2) or 150 seconds (5D5 and mixture containing 5D5). 7A2 scFv was produced in Pichia pastoris. 5D5 Fab used in these experiments is always a tagged version.

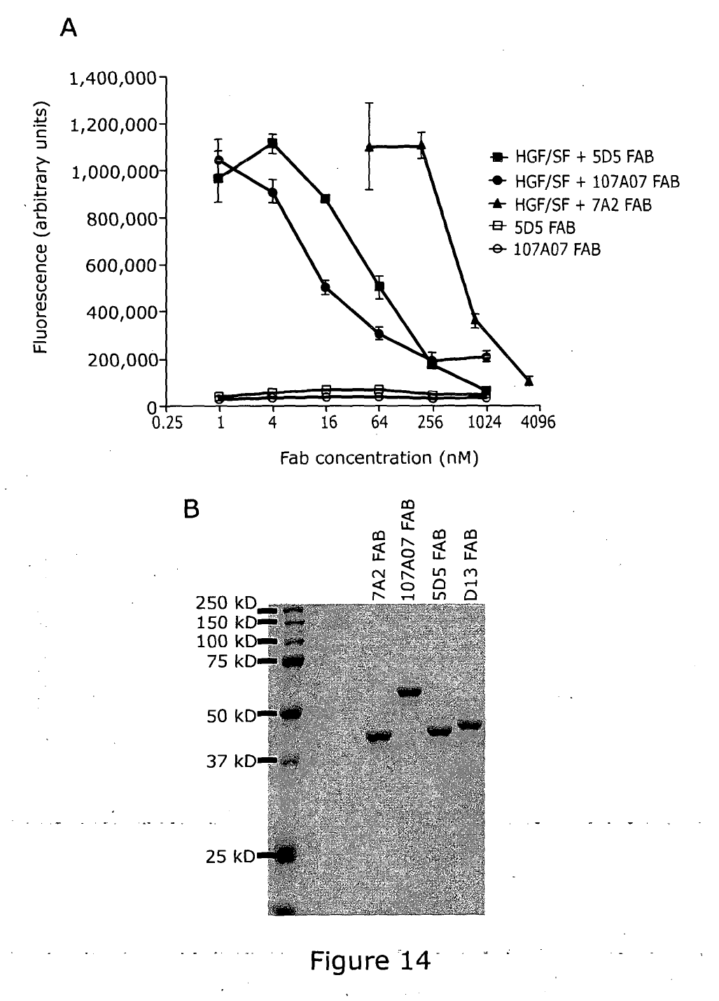

FIG. 14: Migration of fluorescently-labelled SKOV-3 human ovarian cancer cells towards 30 pM HGF/SF is inhibited in the presence of anti-Met Fabs 7A2, 107_A07 and 5D5. 7A2 Fab was tagless and “hinged”, and 107_A07 was tagless and “native” (i.e., not hinged). Little or no migration is observed at a range of Fab concentrations in the absence of HGF/SF. Assays were performed as described in Example 7, below, with the following modification: the number of cells added to each well was reduced to 25,000. Data represent mean±standard deviation of triplicate wells. Data are representative of three independent assays. Fluorescent signal indicates the presence of migrated cells. Part 2 is a Coomassie-stained SDS-PAGE gel in which is loaded 1 μg of 7A2, 107A07, 5D5 and D13 Fabs.

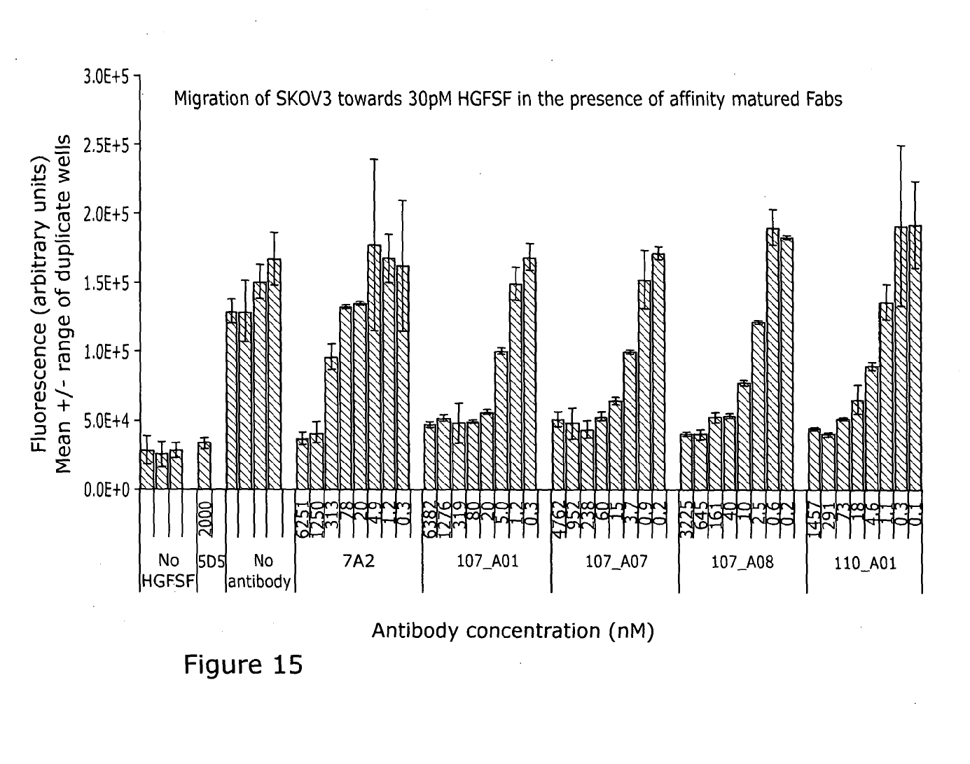

FIG. 15: Migration of fluorescently-labelled SKOV-3 human ovarian cancer cells towards 30 pM HGF/SF is inhibited in the presence of even very high concentrations of anti-Met Fabs 7A2, 107A01, 107_A07, 107A08 and 110A01. All Fabs were in tagged, hinged format. Purified Fabs were quantified by comparison with BSA on an SDS-PAGE gel stained with Sypro Red. Assays were performed as described in Example 7, below. Data represent mean±range of duplicate wells.

FIG. 16: HGF driven proliferation assay of 10,000 BxPC-3 human pancreatic cancer cells. Cells were plated in full media (RPMI supplemented with 10% foetal calf serum) and allowed to adhere overnight before serum starving by washing twice with 0.25% BSA, RPMI and then incubating in the same for 24-48 hours. The media was then replaced with 0.25% BSA, RPMI containing 200 pM HGF/SF and Fabs at the indicated concentrations. 7A2 Fab was tagless and “hinged”, and 107_A07 was tagless and not hinged. After exactly 24 hours incubation, cells were exposed to 10 μM BrdU for 90 minutes. BrdU incorporation was quantitated using a chemiluminescence BrdU incorporation ELISA (Roche) according to the manufacturer's instructions. P: no antibody, N: no HGF/SF. Prior to removal of the BrdU-containing media, all incubations took place at 37° C., 5% CO₂, in a humidified cell culture unit.

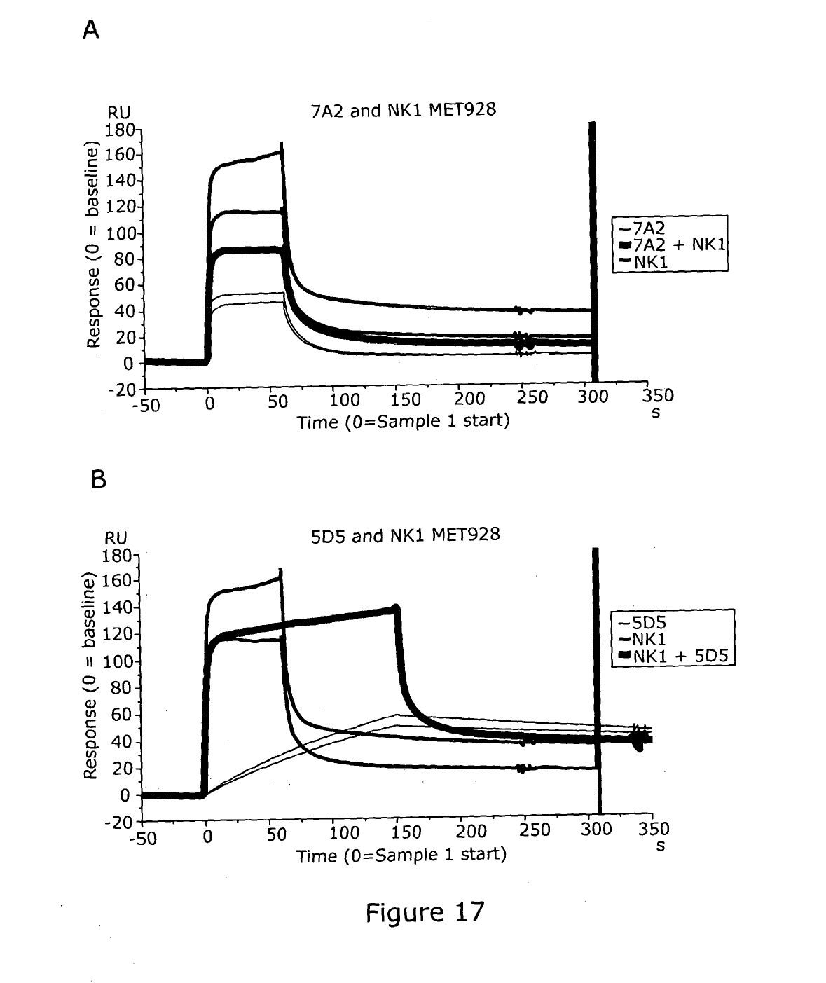

FIG. 17: A CM5 Biacore chip coated with MET928 was exposed to 125 nM and 250 nM 5D5 Fab, 134 nM and 268 nM 7A2 scFv, and 238 nM and 476 nM NK1, or to mixtures thereof. Sensorgrams are shown overlapping and aligned to the start of each injection. Injection times were 60 seconds (7A2, NK1 and 7A2/NK1 mixtures) or 150 seconds (5D5 and 5D5/NK1 mixtures). Part A: Exposure of the chip to 134 nM 7A2 (lower thin line), 268 nM 7A2 (upper thin line), 238 nM NK1 (lower medium-weight line), 476 nM NK1 (upper medium-weight line) and a mixture containing 134 nM 7A2 and 238 nM NK1 (heavy line). Part B: Exposure to 125 nM 5D5 (lower thin line), 250 nM 5D5 (upper thin line), 238 nM NK1 (lower medium-weight line), 476 nM NK1 (upper medium-weight line) or a mixture containing 125 nM 5D5 and 238 nM NK1 (heavy line). NK1 data is the same as in Part A and is shown in both graphs to assist interpretation. 7A2 scFv was produced in Pichia Pastoris.

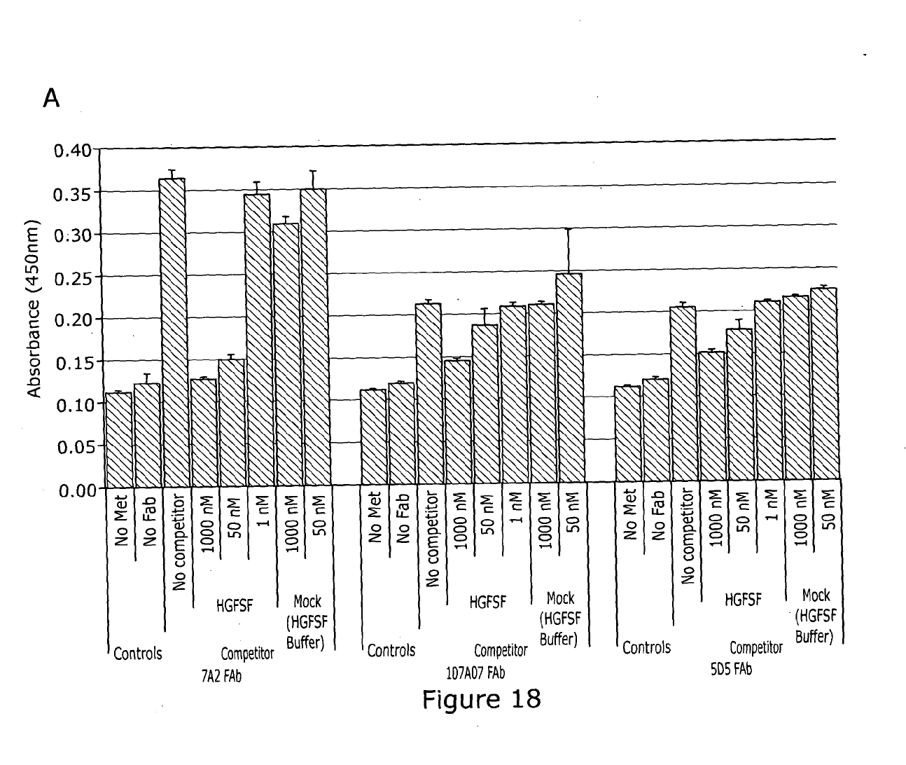

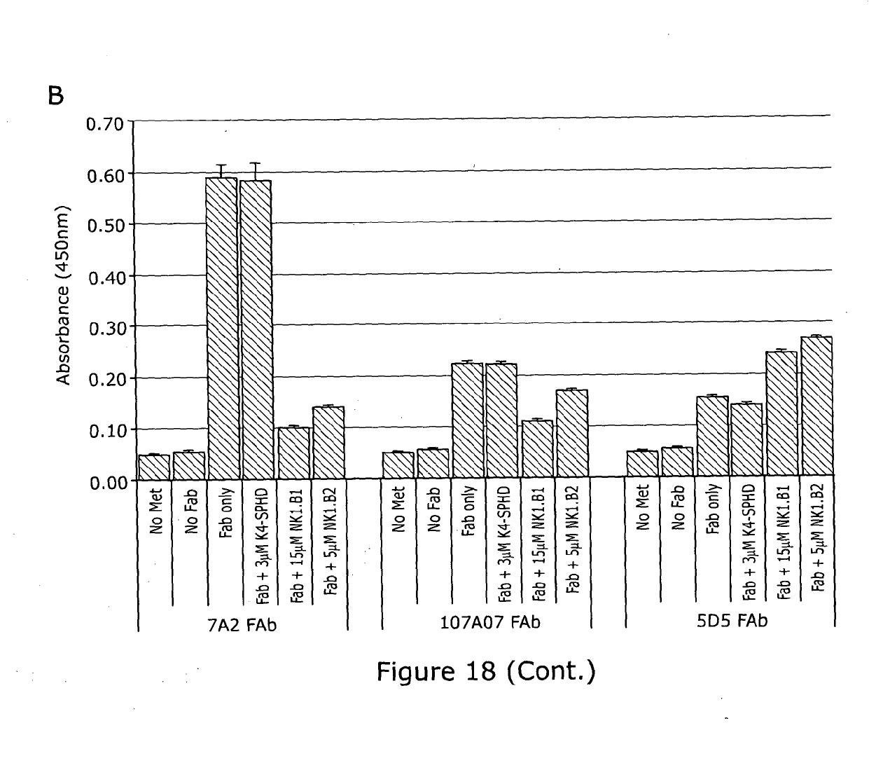

FIG. 18: Competition ELISA demonstrating (A) that HGF/SF competes with 5D5, 7A2 and 107_A07 Fabs for binding to Met extracellular domain and (B) that NK1 competes with 7A2 and 107_A07 Fabs but not with 5D5. 7A2 Fab was tagless and “hinged”, and 107_A07 was tagless and “native” (i.e., not hinged). ELISA plates were coated overnight with 5 μg/ml MET928 and non-specific binding sites blocked with 3% BSA, 0.1% Tween 20 in PBS. MET928-coated plates were exposed to (A) 1.2 nM 7A2, 0.05 nM 107_A07 or 0.03 nM 5D5 in the presence of increasing dilutions of HGF/SF or HGF/SF Buffer (i.e., buffer without HGF/SF), or (B), to 2.5 nM 7A2, 0.1 nM 107_A07 or 0.05 nM 5D5 in the presence of 3 μM K4-SPHD, 15 μM NK1.B1 or 5 μM NK1.B2. NK1 is the major Met-binding fragment of HGF/SF. K4-SPHD indicates an HGF/SF fragment consisting of the fourth Kringle domain and the Serine Proteinase Homology Domain. NK1.B1 and NK1.B2 indicate two different batches of the NK1 protein. Bound Fab was detected with Protein L peroxidase and SureBlue TMB reagent, the reactions stopped with 1N hydrochloric acid and the absorbance at 450 nm quantified on a Polarstar Omega microplate reader. Data represent the mean of triplicate wells; error bars indicate the standard deviation.

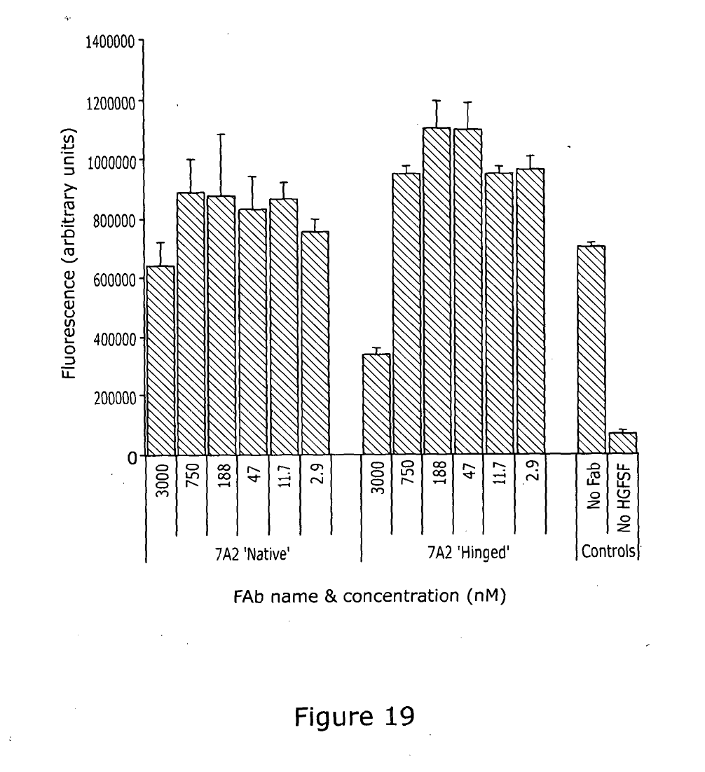

FIG. 19: Migration of fluorescently-labelled SKOV-3 human ovarian cancer cells towards 30 pM HGF/SF is inhibited more effectively by ‘hinged’ 7A2 Fab than by ‘native’ 7A2 Fab. Assays were performed as described in Example 7, below. Data represent mean±standard deviation of triplicate wells.

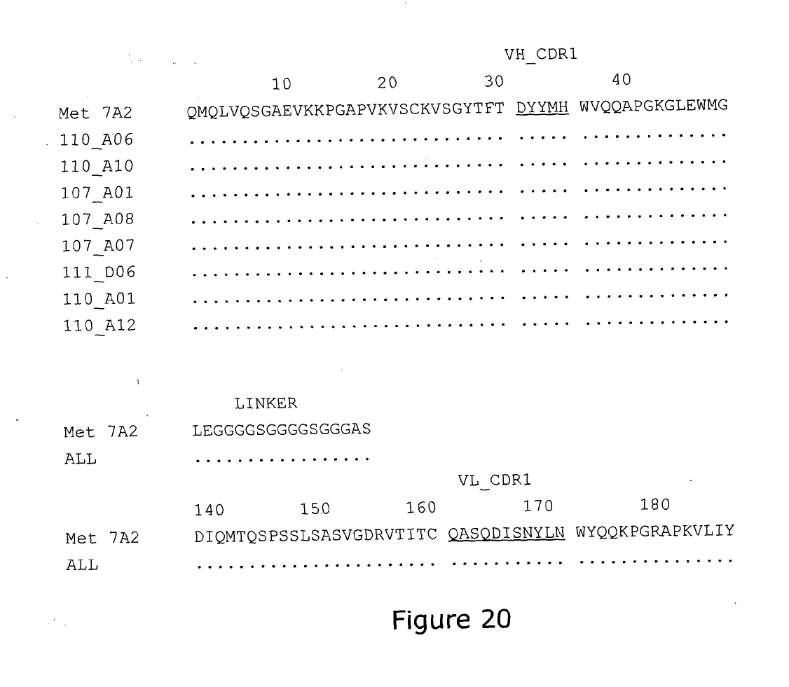

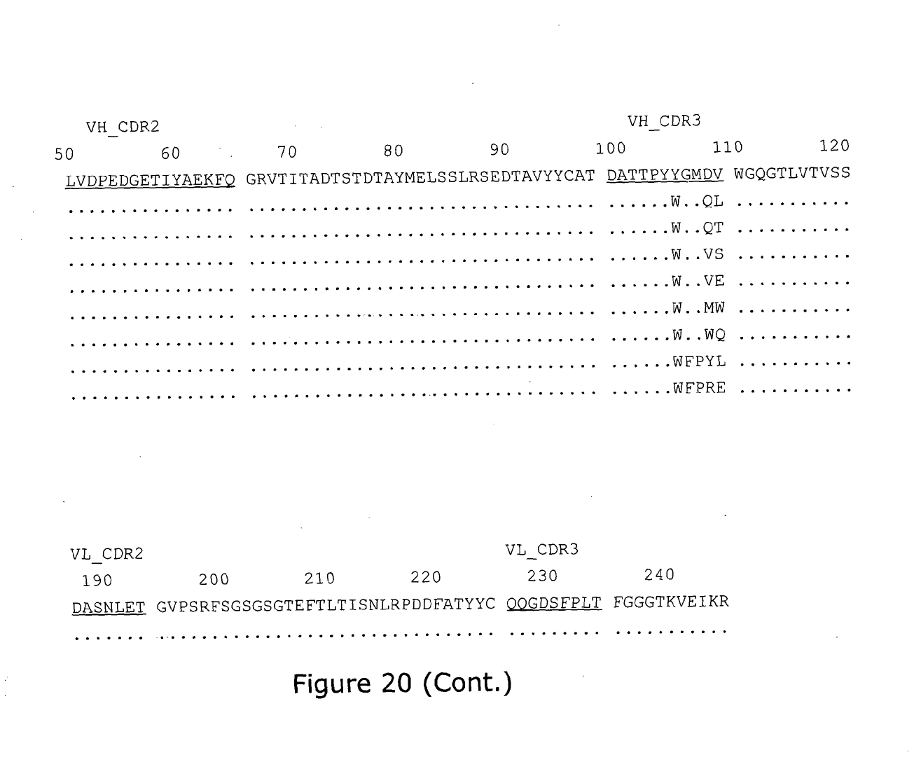

FIG. 20: Sequences of antibodies 107-A08, 107-A07, 111-D06, 107-A01, 110-A10, 110-A06, 110-A12, and 110-A01 with improved affinity. Changes from the amino acid sequence of the parental clone 084-G02 (7A2) are indicated. Various regions of the antibodies are also indicated.

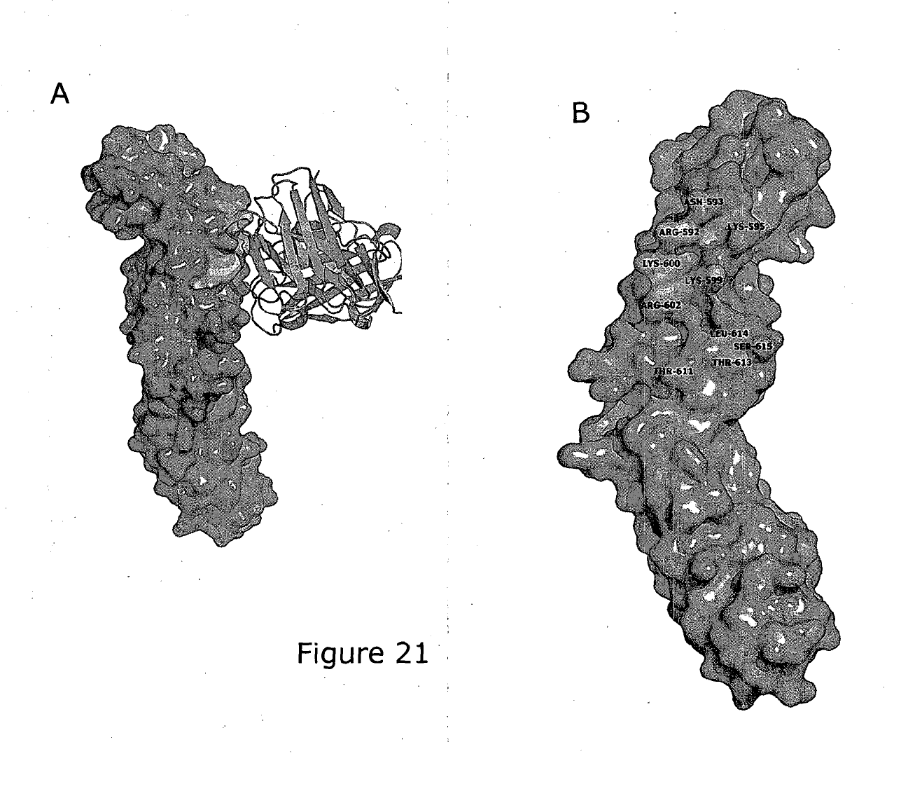

FIG. 21: (A) Structure of the complex between 107_A07 and MET515-741. (B) Surface representation of MET515-741 showing amino acids residues that form interactions with side chain or backbone atoms on 107_A07. The residues are: R592, N593, K595, K599, K600, R602, T611, T613, L614, S615. Residues where the amino acids on human Met differ from mouse Met are highlighted in yellow.

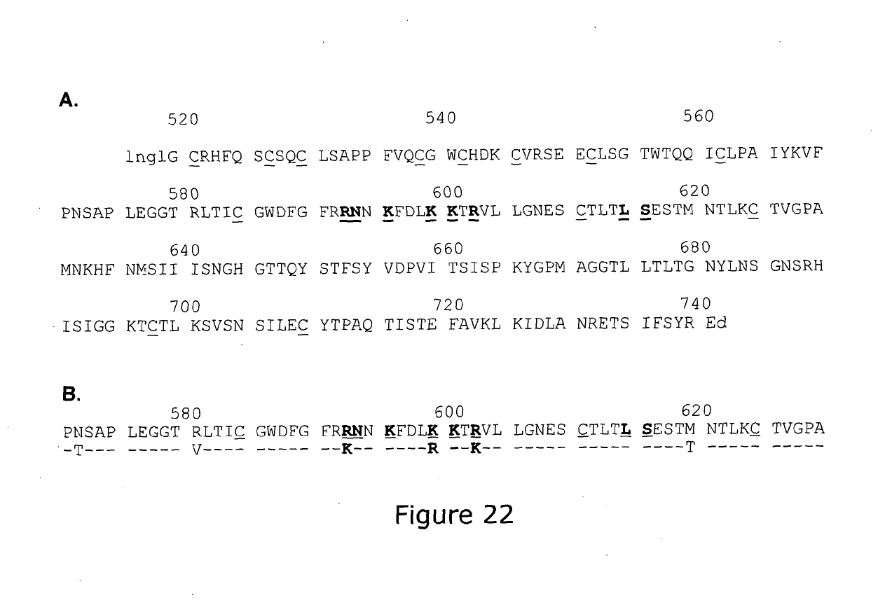

FIG. 22: Sequence of MET515-741 (A) (SEQ ID NO: 33) Non-resolved residues in structure are shown in lower case. Cysteines are shown underlined. X-ray crystallography of the complex of 107_A07 and MET515-574 has identified residues R592, N593, K595, K599, K600, R602, L614, S615 (shown in bold and underlined) as being critical for the interaction with the blocking antibody. (B) Shows a comparison of mouse and human Met (from residues 570-629) (SEQ ID NO: 34) Comparison of the sequence of human versus mouse Met, around the binding site, reveal that conservative amino acid changes occur at 3 of these contact residues. The residues involved are R592K, K599R and R602K (where the first letter indicates the amino acid found in the human sequence, the number represents the residue number and the final letter represents the residue found in the mouse Met sequence).

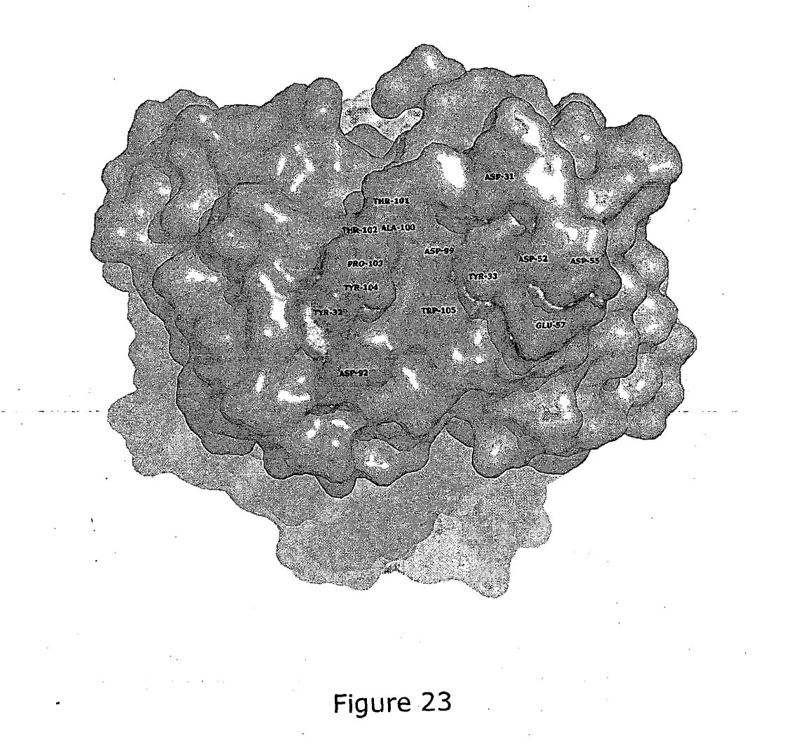

FIG. 23: Surface representation of 107_A07 contacts with Met. Surface representation of antibody showing the foot print of the contact with Met. For the heavy chain (pale cyan) residues 31, 33, 52, 55, 57, 99, 100, 101, 102, 103, 104, 105 (in cyan). For the light chain light chain (light magenta) residues 32 and 92 are involved (magenta).

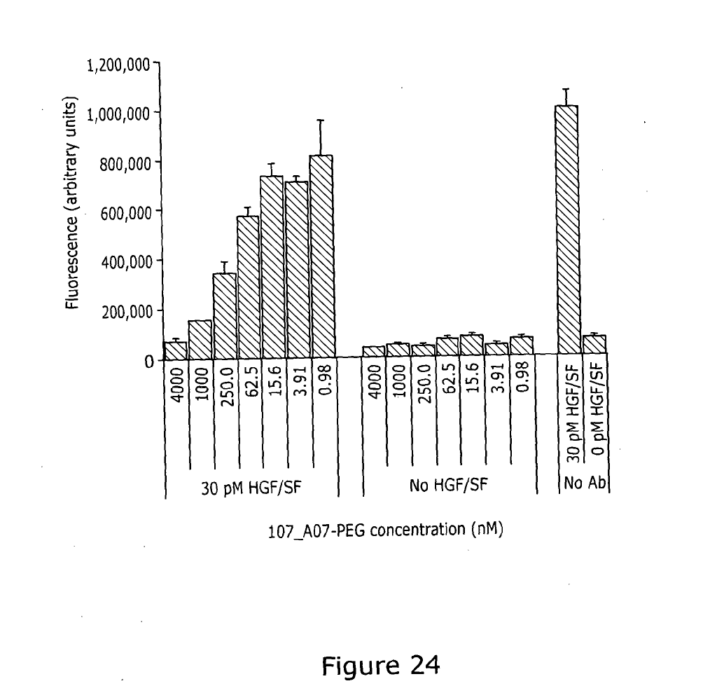

FIG. 24: Migration of fluorescently-labelled SKOV3 cells towards 30 pM HGF/SF in the presence of PEGylated 107_A07 FAb. Data represent mean and standard deviation of at least three wells.

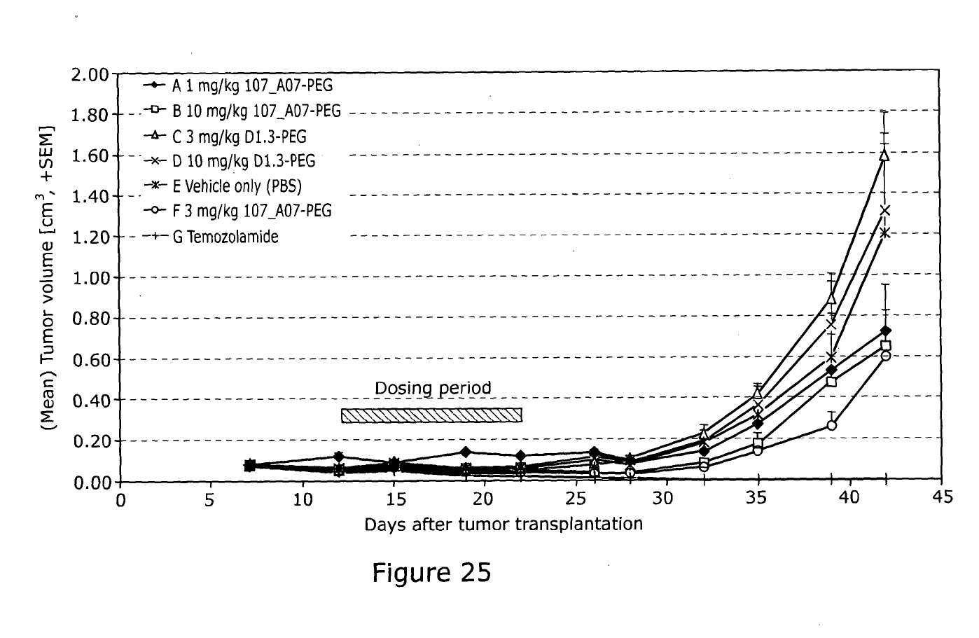

FIG. 25: Effect of 107_A07 Fab on growth of U87 human glioma xenografts. 10⁷ U87 cells were transplanted subcutaneously on NMRI nu/nu mice which were then treated with PEGylated Fabs 107_A07 or huD1.3. Doses of antibody given are indicated. Additional control group G were given Temozolamide p.o. daily for five days starting on Day 7.

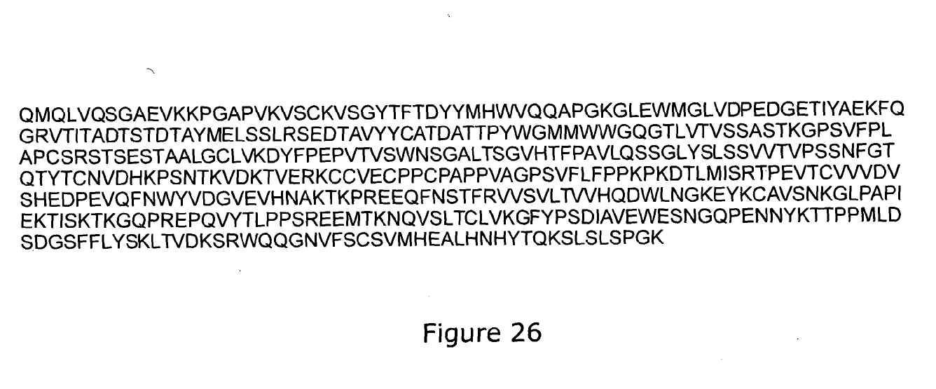

FIG. 26: Sequence of 107_A07 IgG2 heavy chain.

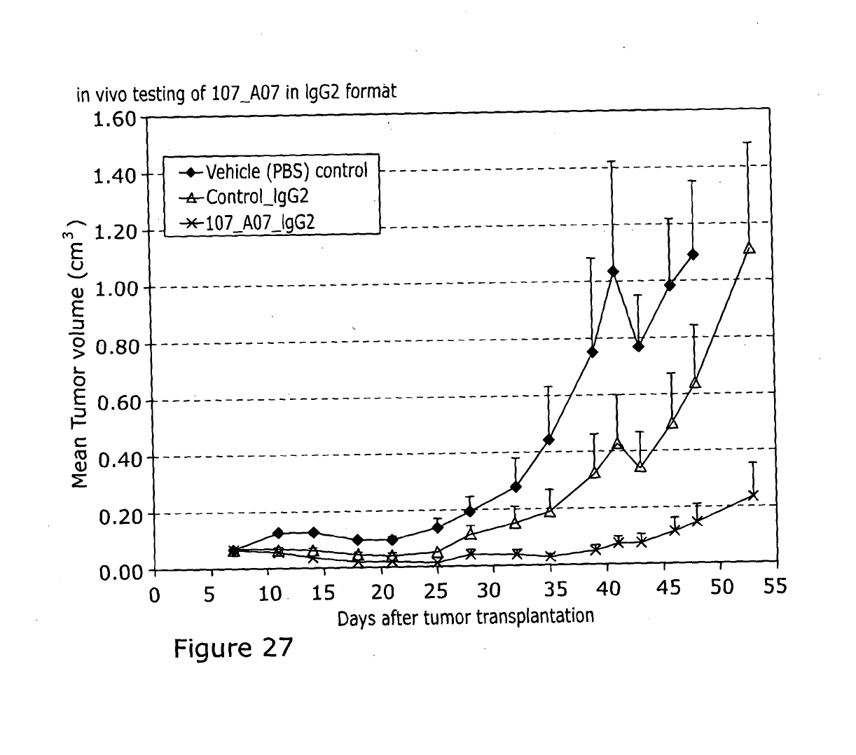

FIG. 27: Effect of 107_A07 in IgG2 format on growth of U87 human glioma xenografts. Groups of 10 female mice of 6-8 weeks age were injected with 10⁷ U87 cells. Twice weekly dosing of Vehicle or test and control antibody at 10 mg/kg was initiated one week later.

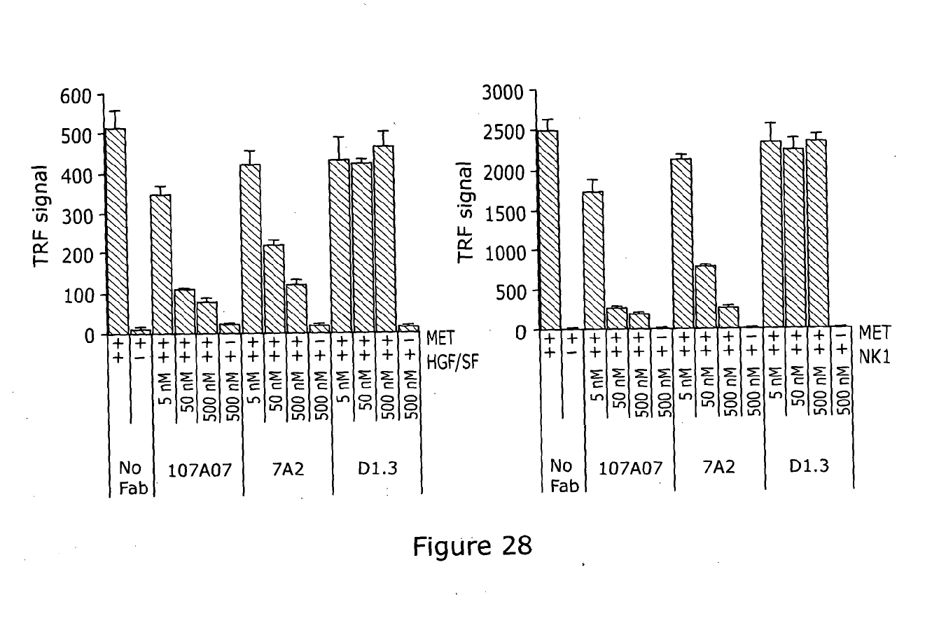

FIG. 28: Binding of FAb-exposed MET928 to plates coated with HGF/SF or the NK1 fragment of HGF/SF. Data represent mean and standard deviation of a minimum of three replicates per sample. Experiment was repeated with similar results.

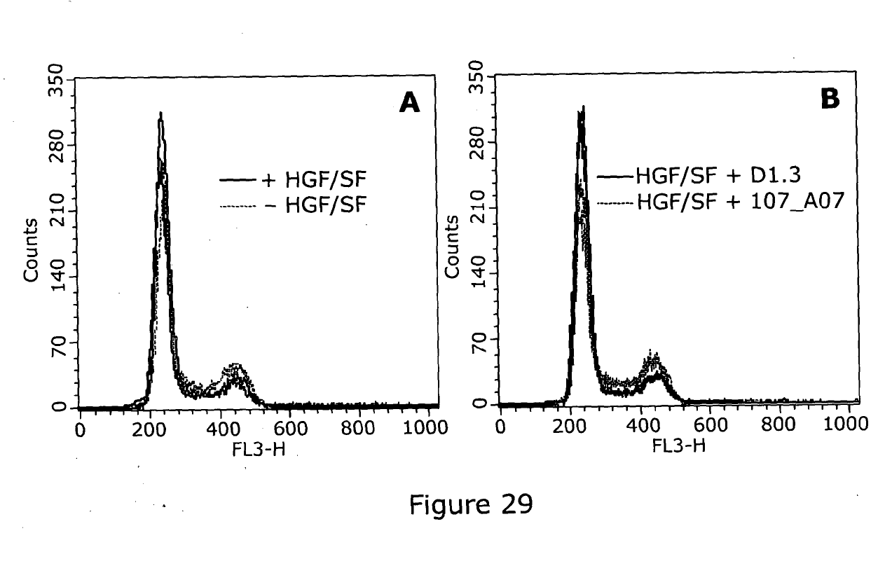

FIG. 29: Cell cycle analysis of U87MG cells exposed to HGF/SF in the presence or absence of 107_A07 FAb. Histograms show overlays of duplicate or triplicate samples as described in Table 24. (A) 300 pM HGF/SF (n=3), 0 pM HGF/SF (n=2); (B): HGF/SF+107_A07 (n=3), HGF/SF+D1.3 (n=3).

### EXAMPLE 1

### Generation of Primary Met Binding Antibodies

Generation of c-Met Antigen for Selection

Constructs available for selection of phage MET antibodies are listed in Table 1 below. The constructs are defined with respect to the amino acid positions, with residues 1-24 encoding a leader sequence. Initial selections were carried out on construct 25-928 produced in Lec 8 cells, which are derivatives of Chinese hamster ovary (CHO) cells deficient in N-linked glycosylation.

TABLE 1
Constructs of the MET ectodomain available
for selection or epitope mapping of phage antibodies

| construct | domain(s) | host cell line |
|---|---|---|
| 25-519 | β-p | NS0, Lec 3.2.8.1 |
| 25-567 | β-p + crd | NS0, Lec 3.2.8.1, |
| 25-741 | β-p + crd + ig1-2 | Lec 3.2.8.1 |
| 25-838 | β-p + crd + ig1-3 | NS0, Lec 3.2.8.1 |
| 25-928 | β-p + crd + ig1-4 | NS0, Lec 8, (Lec 3.2.8.1) |
| 564-932 | ig 1-4 (stalk) | (NS0) |

β-p: β-p-propeller,
crd: cystine-rich domain,
ig: immunoglobulin

The MET amino acid sequence is shown as SEQ ID No: 1, above.

Selection of Primary Antibodies Binding to c-MET

Phage display is a powerful technology for generating antibodies whereby antibody molecules are displayed on the surface of filamentous phage particles which carry the encoding gene. Thus the functional antibody is associated with the encoding gene and antibodies (along with their associated genes) can be selected by panning on immobilised antigen. This involves exposing the library to immobilised antigen, allowing binding to occur, washing away the unbound phage particles and recovering bound phage. Phage-display libraries provide a rich source of antibody diversity, providing potentially hundreds of unique antibodies to a single target (18). Once isolated, the antibody gene can be conveniently shuttled into a variety of expression formats, including whole human IgG molecules expressed in mammalian cells.

Antibodies which bind the extracellular domain of c-MET were selected from the “McCafferty” antibody phage-display library described in Schofield et al (2007). Essentially, genes encoding re-arranged antibody variable heavy (VH) and variable light (VL) chains were amplified from lymphocyte mRNA from non-immunised human donors. VH and VL genes were joined by DNA encoding a flexible peptide to give antibody binding fragments in the form of a single chain Fv (scFv). The cMET protein derived from construct 25-928 (Table 1), hereafter referred to as MET928, was bound to the surface of a plastic tube (Immunosorb tubes from Nunc) for selection. Detailed description for the practice of phage display including antigen coating, library generation, rescue of the initial library and selected population, selection and screening are given in Schofield et al (2007), Pershad et al (2010), and Dyson et al (2011), and is well known in the art.

Two rounds of selection were carried out with the “McCafferty” antibody phage display library. An increase in the number of output phage (represented by the number of Ampicillin resistant colonies) after each round of selection indicates selection of binders is occurring. After each round of selection, the eluted phage were infected into E. coli. In this case, 2.1×10⁴ colonies and 1×10⁷ colonies were derived from rounds 1 and 2 respectively. Polyclonal phage populations were prepared from the selected populations and were tested in ELISA (polyclonal phage ELISA) using ELISA plates coated with MET928. After incubation with phage, plates were washed and bound phage detected using peroxidase conjugated anti-M13 antibodies. FIG. 2 shows that the phage population from only one round of selection binds to MET928 (but not BSA) and the signal is increased in the population arising from two rounds of selection.

Phage particles were also generated from 20 individual colonies from the 2^{nd} round population and 16 of these were shown to be positive in monoclonal phage ELISA.

Identification of Individual Clones with Antagonistic Activity

In order to identify individual clones for further study, the population from two rounds of selection was sub-cloned into pSANG10-3F, an alternative vector optimised for expression of soluble antibody fragments. This construct fuses the C-terminus of the antibody to 6×His/tri-Flag tags (Martin etc., 2006) to facilitate purification and detection of the expressed antibody. DNA encoding the selected antibody scFv was sub-cloned into pSANG10-3F as an Nco/Not1 fragment and individual colonies were picked and grown for screening. Methods for soluble expression of antibody fragments and detection in ELISA have been described previously (Martin et al 2006, Schofield et al 2007, Pershad et al 2010, and Dyson et al 2011). A panel of 186 clones was screened by ELISA to give a group of 76 clones which bound to cMET in ELISA.

The availability of a protein fragment consisting of only the β-propeller domain of c-Met (from construct 25-567, Table 1), hereafter referred to as MET567, permitted mapping of the approximate binding site of the antibodies. Of the 76 positive clones, 14 were specific to the β-propeller in ELISA. This suggests that the other selected antibodies bind to the stalk region of the c-Met protein.

The 76 positive clones were expressed in 50 ml of culture medium using auto-induction medium (Studier et al 2005, and Martin et al 2006) and the expressed protein purified using immobilised metal affinity chromatography (IMAC) on Ni-NTA resin (QIAGEN), The 76 clones were screened in a cell-dispersion assay (scatter assay) using Madin Darby Canine Kidney cells (MDCK cells). This assay assesses the response of target cells to HGF/SF. Of the panel of 76 anti-cMET antibody clones screened, 15 were selected for potential antagonistic activity to the response of target cells to HGF/SF. Antibodies expressed by these clones were also tested for their activity on human cells, using the human pancreatic adenocarcinoma cell line BxPC3 in the HGF/SF scatter assay. Several clones showed partial activity at an antibody concentration of 1 μM.

### EXAMPLE 2

### Affinity Maturation of c-Met Antibodies

As described in Example 1, antibodies with antagonistic activity can be identified by functional screening of populations selected by phage display. The likelihood of success in cancer therapy is increased by having antibodies with optimal affinity/potency. Here we describe the selection of variants with improved affinity using phage display. This involves creation of a mutagenised library of variants from a lead antibody followed by stringent selection using limiting amounts of antigen. A library of variants can be generated by various methods including oligonucleotide directed mutagenesis (see Example 4) and chain shuffling.

### Selection of Variants by Chain-Shuffling

Chain-shuffling involves cloning one chain such as the variable heavy chain (VH) of a selected clone or population, and recombining this with a population of different complementary variable chains (i.e. light chain (VL) partners in the example). Following this “chain shuffling”, stringent selections using limiting amounts of antigens then allow the emergence of new VHNL combinations with superior binding properties to the original.

A chain-shuffled scFv library of 10⁹ clones was constructed using a heavy chain population arising from the two rounds of selection against MET928 described in Example 1. This was generated by PCR and digested with Nco and Xho1 restriction sites which flank the 5′ and 3′ ends respectively of the VH. This was cloned into the Nco1/Xho1 site of the library of human antibody K and A light chains prepared during the initial construction of the “McCafferty” library (described in Schofield et al 2007).

While immobilisation of antigen on plastic surfaces is a convenient means of presenting antigens for antibody selection, the use of soluble, recoverable target e.g. biotinylated antigens, means that the initial binding step can be carried out in solution. Thus, the effective concentration of the antigen may be more closely controlled. Following binding, the complex between biotinylated antigen and phage-antibody is recovered using immobilised streptavidin or streptavidin coated magnetic beads. Selection with soluble biotinylated antigen therefore provides a means to increase the degree of discrimination between clones with closely related affinities within a population e.g. during affinity maturation.

### ‘Affinity-Based’ Selections

Two rounds of selection were carried out using biotinylated Met (prepared through reaction of biotin hydrozide with carbohydrate modifications) and bound phage were recovered on streptavidin beads. The selection was performed over two rounds in reducing concentrations of biotinylated MET928 (FIG. 3A) to provide an increasing stringency of the selection process. In one scheme, the first round of selection was conducted using 10 nM antigen and the second round used 1 nM antigen (S1 10/1). In another scheme, the first and second rounds used 1 and 0.1 nM respectively (S1 1/0.1).

### ‘Ligand Competition’ Selections

In addition to the affinity-based approach, an additional set of selections was performed in parallel in which phage were eluted from the biotinylated MET928 by competition with high concentrations of HGF/SF. We reasoned that that the ligand-competition-based approach may result in an increased proportion of antagonists of HGF/SF and may lead to identification of active antibodies against comparatively rare epitopes, which could be of sufficiently high affinity or could be excellent candidates for subsequent affinity maturation. By utilising both affinity- and ligand-competition-based strategies we aimed to maximise the probability of generating a functionally antagonistic antibody.

Initially, low-stringency screens were performed on either the naïve ‘McCafferty’ library (FIG. 3B) or on the chain-shuffled anti-Met library (FIG. 3C), using 10-100 nM biotinylated antigen as outlined in FIG. 3, in parallel with the ‘affinity-driven’ selections described above. Elution of antigen-bound phage was performed with trypsin according to standard procedures.

A second round of selection was then performed on each of the resulting three phage populations, using 10 nM biotinylated antigen and eluting bound phage by incubation with 2 μM HGF/SF for one hour at room temperature. Eluted phage were amplified by growth in E. coli and purified according to standard procedures. The three populations of purified phage were then subjected to a third round of selection using 1 nM biotinylated antigen and eluting at room temperature with 2 μM HGF/SF. Prior to elution, the mixtures of biotinylated antigen, antigen-bound phage and streptavidin-coated magnetic beads were divided in two. Half of each mixture was incubated with 2 μM HGF/SF for 15 minutes, after which the beads were pelleted and the eluate removed for infection of E. coli. The beads were then resuspended in 2 μM fresh HGF/SF and allowed to incubate for a further 45 minutes before removal of the eluate for further infection of E. coli. Simultaneously, a ‘mock’ elution was performed on the remaining half of the phage-exposed beads, in which beads were exposed to PBS for 15 minutes, followed by replacement with fresh PBS for a further 45 minutes. Both mock- and HGF/SF-eluted beads were then exposed to trypsin for 15 minutes. For analytical purposes, phage-exposed PBS and trypsin were also used to infect E. coli. Table 2 demonstrates that in five out of six cases, more phage were obtained by incubation of the beads with HGF/SF than by incubation with PBS. This suggests that some phage were specifically ‘eluted’ by HGF/SF.

FIG. 3 outlines the eight selection cascades performed.

TABLE 2
Ratio of phage eluted during HGFSF
incubation to phage eluted during PBS incubation

| | 1^{st} elution | 2^{nd} elution | |
|---|---|---|---|
| | HGF/SF | HGF/SF | |
| | or PBS, | or PBS, | 3^{rd} |
| | t0-15 | t15-45 | elution |
| | minutes | minutes | Trypsin |
| Selection cascades 082 & 083 | 5.4 | 2.6 | 0.04 |
| Input: phage from naïve library at 10× | | | |
| concentration | | | |
| Round 1: 100 nM Antigen, trypsin elution | | | |
| Round 2: 10 nM Antigen, HGF/SF elution | | | |
| Round 3: 1 nM Antigen, HGF/SF or PBS | | | |
| elution | | | |
| Selection cascades 084 & 085 | 0.6 | 2.6 | 0.2 |
| Input: phage from shuffled library at 1× | | | |
| concentration | | | |
| Round 1: 100 nM Antigen, trypsin elution | | | |
| Round 2: 10 nM Antigen, HGF/SF elution | | | |
| Round 3: 1 nM Antigen, HGF/SF or PBS | | | |
| elution | | | |
| Selection cascades 086 & 087 | 2.8 | 2.2 | 0.5 |
| Input: phage from shuffled library at 1× | | | |
| concentration | | | |
| Round 1: 10 nM Antigen, trypsin elution | | | |
| Round 2: 10 nM Antigen, HGF/SF elution | | | |
| Round 3: 1 nM Antigen, HGF/SF or PBS | | | |
| elution | | | |

Following selection, an inhibition ELISA using polyclonal phage was used to confirm that the resultant populations of selected antibodies showed significantly increased affinity to MET-928 (FIG. 4). Phage populations arising from two rounds of selection of either the initial library or the chain shuffled libraries were each incubated overnight at 4° C. with a range of concentrations of MET-928 in solution. The sample was then incubated with ELISA plates coated with MET-928 to determine the relative concentration of the remaining unbound phage under each condition. For the chain shuffled populations a lower concentration of MET-928 was required to achieve 50% inhibition of signal indicating a higher average affinity in these populations compared with the original population.

### EXAMPLE 3

### Identification of Functional c-Met Antibodies

Populations from both affinity-based selections and epitope-based selections were cloned into the pSANG10-3F vector (Martin et al, 2006). In total, 960 clones were picked into 96 well plates and grown overnight in auto-induction medium at 30° C. Culture supernatants were analysed by ELISA and more than 60% of them were found to be positive for MET binding.

All clones were then screened directly in an inhibition of cell scatter assay. In the initial screen, culture supernatants from overnight cultures were diluted 1/8 and added directly to BxPC3 cells in the presence of 150-250 pM HGF/SF. Following visual inspection, 51 candidate clones were identified which potentially reduced the scatter induced by HGF/SF. These candidate clones were grown overnight in 500 ml cultures induced with IPTG and the expressed antibody purified from the supernatant using cation exchange (SP Sepharose FF, pH4.5-5) and IMAC using Ni-NTA resin (QIAGEN).

Purified antibody was then tested for both agonistic and antagonistic activity in a quantitative migration assay using human ovarian cancer cells SKOV-3 cells. Analysis of a selected 17 clones is shown in FIG. 5. This assay measures inhibition by antibodies of the HGF/SF induced migration of SKOV-3 from the upper to the lower chamber of a Boyden chamber, and is described in Example 7, below. In FIG. 5B the antibodies were all tested in the SKOV-3 migration assay at a concentration of 1 μM of antibody and clone 7A2 was identified as the most active antibody.

One of the antibodies identified from this screen with HGF/SF blocking activity was antibody 7A2. Antibody 7A2 (also known as 084-G02) was derived from the chain shuffled library and had been selected using biotinylated antigen and competitive elution with HGF/SF (selection cascade P7 (084) in FIG. 3).

7A2 was chosen for further study since it had higher affinity and bound to a distinct epitope from the 5D5 antibody produced by Genentech (see below). 7A2 showed better inhibition than all the other clones screened, giving strong inhibition of HGF/SF induced migration of SKOV cells. Further analysis showed that the 7A2 scFv had an IC₅₀ of 2.3 nM in the SKOV migration assay (FIG. 6 and Table 3). The sequence of 7A2 is shown in FIG. 7.

TABLE 3
Inhibition of HGF/SF-induced migration of
SKOV-3 cells by 7A2 and 5D5 scFv: IC₅₀ (nM) data.

| | 7A2 | 5D5 |
|---|---|---|
| Assay 1 | 2.1 | 115.5 |
| Assay 2 | 2.5 | 83.6 |
| Mean | 2.3 | 99.6 |

GraphPad Prism software was used to fit sigmoidal dose-response curves. Curve ends were constrained to migration values in the absence of antibody (top) and the absence of HGF/SF (bottom) for each assay.

### EXAMPLE 4

### Mutagenesis and Affinity Maturation of the Antibody 7A2

Mutagenesis and selections were carried out on 7A2 to improve the affinity further.

The CDR3 is the most diverse part of the antibody variable domain and occupies a central region in the binding surface. In 7A2, there are 11 amino acid residues in CDR3 of the heavy chain and 9 amino residues in CDR3 of the light chain. Oligonucleotide-mediated site-directed mutagenesis was used to construct five derivative libraries which are diversified around the CDR3 of the Heavy and Light chain variable domains.

Three Heavy chain libraries and two Light chain libraries were constructed using PCR to construct intact antibody genes by PCR assembly (FIG. 8).

Mutational oligonucleotides were synthesised with a stretch of 5-6 randomised codons flanked by fixed sequences with a complete match to 7A2. In the randomised regions the original codon was replaced with NNS (where N is any nucleotide and S is C or G). These 32 codons encode all 20 amino acids and one (amber) stop codon. The DNA sequence of the mutagenic oligos is shown in FIG. 8, and the position relative to the protein sequence of 7A2 is also shown with the randomised positions represented by an “X”. A plasmid containing 7A2 was used as a template and the oligonucleotides VH3.1, VH3.2 or VH3.3 were each used as a 5′end PCR primer along with a primer based in vector downstream of the 7A2. The resultant three PCR products (product 1) then encodes the 3′ end of VH framework 3 (VH FR3), a randomised VH CDR3, VH framework 4, linker sequence and the complete VL gene. Another two PCR products (product 2) were generated encoding the remainder of the 7A2 heavy chain (from framework) to framework 3) by using a 5′ end primer based in the vector upstream of 7A2 and the two “reconstruction primers” shown in FIG. 8. The second PCR product has a region of overlap with the first PCR product in the VH FR3 region. This overlap region was used to drive an assembly between product 1 and product 2 in an assembly PCR reaction using the 5′ end vector based primers of product 1 and the 3′ end vector based primer of product 2 to amplify the assembled product. The three resulting assembled products, encoding a population of scFv genes with an internal randomised region in CDR3 in three different positions were then digested with Nco1 and Not 1 (used in the original cloning of 7A2) and the populations were cloned into pSANG4 (Schofield et al, 2007). A similar approach was used to randomise the CDR3 region of the light chain and create 2 mutagenised libraries as shown in FIG. 8.

Randomisation of 5 or 6 residues will result in 32 and 32⁶ possible combinations respectively (3.3×10⁷-10⁹ clones). Although many clones will lose binding activity, most/all combinations can be sampled by phage antibody display, enabling identification of mutants with improved affinity. The different libraries created using the three different heavy chain mutant oligos were combined into a single heavy chain library. The different libraries created using the two different light chain mutant oligos were combined into a single light chain library (“secondary mutagenised library”).

Although randomisation of CDRs has the potential to introduce beneficial amino acids which enhance binding to the target antigen there is also the potential to reduce or abolish antigen recognition. Our strategy was to conduct a low stringency selection to enrich for clones which retained binding to MET from the secondary mutagenised libraries and then to combine the selected heavy and light chain populations into a combined shuffled mutant library, with randomisation in both CDR3s (“tertiary shuffled library”), for further stringent selection.

Two rounds of selection were carried out on the secondary mutagenised heavy chain and light chain libraries using 100 nM biotinylated Met in solution, prior to capture of bound phage on streptavidin magnetic beads. Phage ELISAs (using streptavidin coated plates to which 2 ug/ml biotinylated antigen was bound) indicated that 90% of clones were positive at both round 1 and round 2. As little difference was evident between the round one and round two selections, the round one selected population, which had output numbers of 1-5×10⁷ was used to create a VH/VL shuffled library in order to maintain diversity, prior to shuffling.

### Construction and Selection of “Tertiary Shuffled Library”

The populations arising from one round of selection of the heavy and light chain oligonucleotide mutagenised secondary libraries were combined into a “tertiary shuffled library”. This was constructed by excising the original VH gene from the selected light chain repertoire and replacing it with the entire VH repertoire selected from the oligo mutagenised heavy chain library. The resulting tertiary library which combined randomised residues in both heavy and light chain CDRs contained 1.7×10⁹ clones.

Phage particles were rescued from the tertiary library and PEG precipitated using standard methods. 100 μl of phage-antibodies at a concentration of 10× relative to that found in the unprecipitated culture were used for the first round of selection (approximately 5×10¹¹ phage/selection) and non-precipitated phage (1×) were used for subsequent rounds. Stringency of selection can be increased by reducing the amount of antigen used, although the optimal concentration needs to be determined empirically. Rescued phage were allowed to bind to antigen at concentrations ranging from 100 nM to 100 pM and the output numbers after selection were compared with the “no antigen” control. Selection conditions yielding 5-10× fold more output phage than the “no antigen” control were used for subsequent rounds. Using 1 nM antigen at round 1, output numbers were 10 fold above background. Rescued phage from this population were used for a second round of selection. In a similar way, based on output numbers, selections were carried out as shown in Tables 4-7.

### Round 1 Selection

TABLE 4
Input phage from shuffled library at 10× concentration

| Antigen concentration | Number of selected clones |
|---|---|
| 100 nM | 1 × 10⁹ |
| 10 nM | 1 × 10⁹ |
| 1 nM | 8 × 10⁷ |
| 100 pM | 8 × 10⁶ |
| 0 nM | 8 × 10⁶ |

### Round 2 Selection

TABLE 5
1× Input phage = 1 nM output from round 1 at 1× concentration

| Antigen concentration | Number of selected clones |
|---|---|
| 1 nM | 1.2 × 10⁸ |
| 100 pM | 1.2 × 10⁷ |
| 10 pM | 2 × 10⁶ |
| 1 pM | 2 × 10⁶ |
| Zero antigen control | 4 × 10⁵ |

### Round 3 Selection

TABLE 6
1× Input phage = 1 nM output from round 2

| Antigen concentration | Number of selected clones |
|---|---|
| 1 nM | 3.2 × 10⁸ |
| 100 pM | 2 × 10⁷ (selection no 109) |
| 10 pM | 5 × 10⁶ (selection no 110) |
| 1 pM | 5.6 × 10⁵ |
| Zero antigen control | 1.8 × 10⁵ |

TABLE 7
Input phage = 100 pM output from round 2

| Antigen concentration | Number of selected clones |
|---|---|
| 1 nM | 6.4 × 10⁸ |
| 100 pM | 8 × 10⁷ (selection no 107) |
| 10 pM | 5 × 10⁶ (selection no 108) |
| 1 pM | 1 × 10⁶ |
| Zero antigen control | 2 × 10⁵ |

A third round of selection combined with an extended off rate selection was also carried out. For the off-rate selection the standard washing process (three washes in 1 ml aliquots of PBS buffer with 0.1% Tween 20 and two washes in 1 ml aliquots of PBS) was used to remove all non-bound phage and the beads with retained phage were then added to a solution of 33 nM non-biotinylated antigen. Those phage with faster off rates will dissociate from the bound antigen more readily and the non biotinylated antigen in solution will reduce re-binding. Output numbers are provided in Table 8 below.

TABLE 8
Output numbers from off-rate selection

| | Washes | Number of selected clones |
|---|---|---|
| Zero | None - standard selection and | 1 × 10⁹ |
| | standard wash process only | |
| 1 hour | 1 × 1 hr wash | 4.6 × 10⁸ |
| 2 hours | 2 × 1 hr wash | 4 × 10⁸ |
| 3 hours | 3 × 1 hr wash | 4.5 × 10⁸ |
| 4 hours | 4 × 1 hr wash | 7 × 10⁸ (selection no 111) |
| 7 hours | 4 × 1 hr wash + 1 × 3 hr wash | 2.6 × 10⁸ |
| 10 hours | 4 × 1 hr wash plus | 2.7 × 10⁸ (selection no 112) |
| | 2 × 3 hr wash | |
| 13 hours | 4 × 1 hr wash + 3 × 3 hr wash | 2.6 × 10⁸ |
| 16 hours | 4 × 1 hr wash + 4 × 3 hr wash | 2.7 × 10⁸ |

The selections which utilised 100 pM and 10 pM antigen at round 3 and the populations derived from the 4 and 10 hours of off rate selection were sub-cloned into the expression vector pSANG10-3F for affinity and sequence analysis. These populations were given a selection number, and this number was used to name clones derived from that selection e.g. clone 110_{—}2H1 came from selection 110 which used 1 nM, 1 nM and 10 pM biotinylated Met928 in rounds 1, 2 and 3 respectively.

DNA encoding the selected scFv populations was sub cloned into the expression vector pSANG10-3F (Martin etc., 2006) and individual colonies picked and grown. A total of 1152 clones from these selections were screened by ELISA and sequenced. This identified 146 clones with unique sequences that were positive in ELISA. All of these clones had mutations to the Heavy chain CDR3, but no mutations in the Light chain CDR3. These clones were picked into a new glycerol stock plate, compiled into 2 master 96 well plates and the clones renamed, based on the position within that plate (see Table 9).

TABLE 9
Panel of 146 affinity matured clones derived
from 7A2 (also known as 084-G02) showing
their heavy chain CDR3 amino sequences.

| | Plate 1 | | Plate 2 |
|---|---|---|---|
| Clone | VH CDR3 | Clone | VH CDR3 |
| name | sequence | name | sequence |
| 107_A1 | DATTPYWGMVS | 109_H7 | DATTPYWFPRW |
| 107_A10 | DATTPYWGMQE | 109_H8 | DATTPYWFPRS |
| 107_A11 | DATTPYWGMAW | 109_H9 | DATTPYWGMTK |
| 107_A12 | DATTPYWWMAK | 110_A1 | DATTPYWFPYL |
| 107_A2 | DATTPYWGMQS | 110_A10 | DATTPYWGMQT |
| 107_A3 | DATTAYWGMQK | 110_A12 | DATTPYWFPRE |
| 107_A4 | DATTPYWFPYH | 110_A2 | DATTPYWGMST |
| 107_A5 | DATTPYWGMSK | 110_A3 | DATTPYWGVQV |
| 107_A6 | DATTPYWTPFV | 110_A5 | DATTPYWYPFV |
| 107_A7 | DATTPYWGMMW | 110_A6 | DATTPYWGMQL |
| 107_A8 | DATTPYWGMVE | 110_A8 | DATTPYWCMAI |
| 107_A9 | DATTPYWGMMT | 110_A9 | DATTPYWGMAG |
| 107_B1 | DATHIYWNMDI | 110_B1 | DATTPYWGMBL |
| 107_B10 | DATTPYWGMRV | 110_B10 | DATTPYWGMBM |
| 107_B11 | DATTPYWGMAM | 110_B3 | DMELNYYGMDV |
| 107_B12 | DATTPYWGMTA | 110_84 | DATTPYWFPBT |
| 107_B2 | DATTPYWGMQM | 110_B5 | DATTPYWGMMS |
| 107_B3 | DATTPYWGMKM | 110_B6 | DTDLKYYGMDV |
| 107_B4 | DATTPYWFPNV | 110_B7 | DGLWMYYGMDV |
| 107_B5 | DATTPYWGMQI | 110_B8 | DATTPYWGMML |
| 107_B6 | DATTPYWGMQK | 110_B9 | DATTPYWSPIW |
| 107_B7 | DATTPYWCMRA | 111_B12 | DATTPYWGAQA |
| 107_B8 | DATTPYWGMAE | 111_C1 | DATTPYWSPWA |
| 107_C1 | DATTPYWGMSM | 111_C10 | DATTPYWTPEI |
| 107_C2 | DATTPYWGMVK | 111_C12 | DATTPYWFPRY |
| 107_C4 | DATTPYWSPWM | 111_C3 | DATTPYWGMQG |
| 107_C5 | DATTPYWYPIV | 111_C5 | DATTPYWFPKV |
| 107_C6 | DATTPYWGMSV | 111_C6 | DATTPYWFPQH |
| 108_C11 | DATTPYWFPII | 111_C8 | DATTPYWFPVV |
| 108_C12 | DATTPYWGMAS | 111_C9 | DATTPYWGMMA |
| 108_C8 | DATTPYWFPYI | 111_D1 | DATTPYWGMDE |
| 108_C9 | DATTPYWFPRB | 111_D11 | DATTPYWGMMR |
| 108_D1 | DATTPYWTPWK | 111_D2 | DATTPYWFPWI |
| 108_D10 | DATTPYWGMSH | 111_D3 | DATTPYWGAAL |
| 108_D11 | DATTPYWGAAA | 111_D4 | DATTPYWGMWV |
| 108_D12 | DATTPYWTPYV | 111_D5 | DATTPYWFPAV |
| 108_D2 | DATTPYWAPWV | 111_D6 | DATTPYWGMWQ |
| 108_D3 | DATTPYWFPYV | 111_D7 | DATTPYWFPYA |
| 108_D4 | DATTPYWGMSG | 111_D8 | DATTPYWGMSW |
| 108_D5 | DATTPYWSPHT | 111_E10 | DATTPYWYPYA |
| 108_D7 | DATTPYWGMDM | 111_E11 | DATTPYWGMRE |
| 108_D9 | DATTPYWYPYV | 111_E12 | DATTPYWSPWV |
| 108_E1 | DATTPYWFPLV | 111_E3 | DATTPYWFPVE |
| 108_E10 | DATTPYWFPWV | 111_E4 | DATTPYWGIEV |
| 108_E11 | DATTPYCSRTS | 111_E5 | DATTPYWGMCE |
| 108_E12 | DATTPYWLAAE | 111_E6 | DATTPYWGMAV |
| 108 E2 | DATTPYWTPWV | 111_E7 | DATTPYWGMMM |
| 108_E4 | DATTPYWGMIW | 111_E8 | DATTPYWFPYR |
| 108_E5 | DATTPYWGMWI | 112_F1 | DATTPYWGMAR |
| 108_E7 | DATTPYWGMWR | 112_F11 | DATTPYWGMMQ |
| 108_E8 | DATTPYWWMQK | 112_F12 | DATTPYWGMQW |
| 108_F1 | DATTPYWFPRF | 112_F4 | DATTPYWGMAD |
| 108_F11 | DGSNLSWQMDV | 112_F8 | DATTPYWTPFL |
| 108_F2 | DATTPYWGMSS | 112_F9 | DATTPYWTWV |
| 108_F3 | DATTPYWGMAK | 112_G1 | DATTPYWGMWH |
| 108_F6 | DATTPYCCTIE | 112_G11 | DATTPYWFPYT |
| 108_G1 | DATTPYWFPRM | 112_G3 | DATTPYWGMSA |
| 108_G3 | DATTPYWAPWR | 112_G4 | DATTPYWGAQS |
| 108_G4 | DATTPYWFPRF | 112_G5 | DATTPYWGMRS |
| 108_G5 | DATTPYWWMQS | 112_G6 | DATTPYWFPFV |
| 108_G6 | DATTPYWFPVW | 112_G7 | DATTPYWFPQT |
| 109_G10 | DATTPYWFPRV | 112_G9 | DATTPYWGMQR |
| 109_G11 | DATTPYWWTAN | 112_H1 | DATTPYWGAAV |
| 109_G12 | DATTPYWGMAA | 112_H10 | DATTPYWGIRK |
| 109_G8 | DATTPYWGMAT | 112_H11 | DATTPYWFPQV |
| 109_G9 | DATTPYWGMQE | 112_H12 | DATTPYWFPYK |
| 109_H1 | DATTPYWGVQK | 112_H2 | DATTPYWGMQV |
| 109_H11 | DATTPYWGMAQ | 112_H4 | DATTPYWGMWK |
| 109_H12 | DATTPYYGMDV | 112_H5 | DATTPYWGMQA |
| 109 H2 | DATTPYWGMWE | 112_H6 | DATTPYWGMME |
| 109_H3 | DATTPYWGMQQ | 112_H7 | DATTPYWGVQS |
| 109_H5 | DATTPYWGAQE | 112_H8 | DATTPYWLPLY |
| 109_H6 | DATTPYWFPRT | 112_H9 | DATTPYWFPYS |

These are heavy chain CDR3 sequences of antibody clones that bind to MET, and thus have potential utility in the methods, uses, compositions and compounds of the present invention. For example, antibodies that bind MET having these CDR3 sequences may be useful in identifying, antagonising the function of, detecting and purifying MET.

Single chain antibodies from all 146 positive clones were expressed in 10 ml of culture medium using auto-induction medium (Studier et al 2005, Martin et al 2006) and the expressed protein purified using IMAC on Ni-NTA resin (QIAGEN). The purified clones were then screened by surface plasmon resonance to identify those clones with slower off rates.

A panel of 8 mutant scFv antibodies having higher affinity than the parental 7A2 antibody was chosen for further study. These clones were colony purified and new glycerol stocks prepared before re-sequencing (sequences are shown in Tables 10 and 11, and in FIG. 20).

TABLE 10
Heavy chain amino acid sequences of heavy
chain CDR3 mutants with improved affinity.

| Clone | Sequence |
|---|---|
| >107-A08 | QMQLVQSGAEVKKPGAPVKVSCKVSGYTFTDYYMHW |
| | VQQAPGKGLEWMGLVDPEDGETIYAEKFQGRVTITA |
| | DTSTDTAYMELSSLRSEDTAVYYCATDATTPYWGMV |
| | EWGQGTLVTVSS (SEQ ID No: 17) |
| >107-A07 | QMQLVQSGAEVKKPGAPVKVSCKVSGYTFTDYYMHW |
| | VQQAPGKGLEWMGLVDPEDGETIYAEKFQGRVTITA |
| | DTSTDTAYMELSSLRSEDTAVYYCATDATTPYWGMM |
| | WWGQGTLVTVSS (SEQ ID No: 18) |
| >111-D06 | QMQLVQSGAEVKKPGAPVKVSCKVSGYTFTDYYMHW |
| | VQQAPGKGLEWMGLVDPEDGETIYAEKFQGRVTITA |
| | DTSTDTAYMELSSLRSEDTAVYYCATDATTPYWGMW |
| | QWGQGTLVTVSS (SEQ ID No: 19) |
| >107-A01 | QMQLVQSGAEVKKPGAPVKVSCKVSGYTFTDYYMHW |
| | VQQAPGKGLEWMGLVDPEDGETIYAEKFQGRVTITA |
| | DTSTDTAYMELSSLRSEDTAVYYCATDATTPYWGMV |
| | SWGQGTLVTVSS (SEQ ID No: 20) |
| >110-A10 | QMQLVQSGAEVKKPGAPVKVSCKVSGYTFTDYYMHW |
| | VQQAPGKGLEWMGLVDPEDGETIYAEKFQGRVTITA |
| | DTSTDTAYMELSSLRSEDTAVYYCATDATTPYWGMQ |
| | TWGQGTLVTVSS (SEQ ID No: 21) |
| >110-A06 | QMQLVQSGAEVKKPGAPVKVSCKVSGYTFTDYYMHW |
| | VQQAPGKGLEWMGLVDPEDGETIYAEKFQGRVTITA |
| | DTSTDTAYMELSSLRSEDTAVYYCATDATTPYWGMQ |
| | LWGQGTLVTVSS (SEQ ID No: 22) |
| >110-A12 | QMQLVQSGAEVKKPGAPVKVSCKVSGYTFTDYYMHW |
| | VQQAPGKGLEWMGLVDPEDGETIYAEKFQGRVTITA |
| | DTSTDTAYMELSSLRSEDTAVYYCATDATTPYWFPR |
| | EWGQGTLVTVSS (SEQ ID No: 23) |
| >110-A01 | QMQLVQSGAEVKKPGAPVKVSCKVSGYTFTDYYMHW |
| | VQQAPGKGLEWMGLVDPEDGETIYAEKFQGRVTITA |
| | DTSTDTAYMELSSLRSEDTAVYYCATDATTPYWFPY |
| | LWGQGTLVTVSS (SEQ ID No: 24) |

TABLE 11
Heavy chain CDR3 amino acid sequences
of mutants with improved affinity.

| | |
|---|---|
| 084-G02(7A2) | DATTPYYGMDV (SEQ ID No: 4) |
| 107-A08 | DATTPYWGMVE (SEQ ID No: 5) |
| 107-A07 | DATTPYWGMMW (SEQ ID No: 6) |
| 111-D06 | DATTPYWGMWQ (SEQ ID No: 7) |
| 107-A01 | DATTPYWGMVS (SEQ ID No: 8) |
| 110-A10 | DATTPYWGMQT (SEQ ID No: 9) |
| 110-A06 | DATTPYWGMQL (SEQ ID No: 10) |
| 110-A12 | DATTPYWFPRE (SEQ ID No: 11) |
| 110-A01 | DATTPYWFPYL (SEQ ID No: 12) |

### EXAMPLE 5

### Synthesis and Expression of 5D5

The Genentech human anti-MET antibody 5D5 was used as a control during the screening and analysis of antibodies selected from our human antibody library. The amino acid sequence of antibody 5D5 was taken from U.S. Pat. No. 7,476,724 B2, and the gene was synthesised by GeneArt. The synthesised gene was constructed with restriction sites which allowed us to clone it as a scFv antibody into the vectors pSANG4 and pSANG10 (Schofield et al, 2007), and as a Fab into the vectors pBioCam3-3F for heavy chain expression and pBioCam1-3F for light chain expression.

For expression of 5D5 scFv antibody protein, the 5D5 clone in pSANG10 in BL21-DE3 pRARE cells (Novagen) was expressed in auto-induction medium (Studier et al, 2005; Martin et al, 2006) and the expressed protein purified using IMAC on Ni-NTA resin (QIAGEN).

For expression of 5D5 Fab antibody protein, the 5D5 heavy and light chain constructs in the vectors pBioCam3-3F and pBioCam1-3F were transfected into HEK293F cells Transfection and growth of HEK293 cells was performed using standard methods cells (Tom et al, 2007; Backiwal et al, 2008; Wulhfard et al, 2010). The purified 5D5 plasmid DNA was complexed with PEI for transfection and the cells were grown in either Gibco Freestyle media either serum free or with 10% serum added. The expressed protein was purified using IMAC on Ni-NTA resin (QIAGEN).

### EXAMPLE 6

### Conversion of 7A2 and Affinity Matured Variants into Fab Format

While the scFv format is a convenient antibody format for phage display there is the possibility that dimeric forms can be created for some clones through the pairing of VHs and VL in trans across two different scFv chains. This dimerisation potentially leads to problems through agonism of the Met receptor and also causes complications in measuring affinity. It would be desirable to have the antibody formatted as Fab which will circumvent problems of variable dimerisation. This is also a more standardised form for in vivo experiments.

Several expression vectors were constructed which facilitates the conversion of antibodies from the “McCafferty” library into a Fab format for expression in mammalian cells. In this library VH genes are flanked by Nco1 and Xho1 sites at the 5′ and 3′ ends respectively. VL genes are flanked by Nhe1 and Not1 sites at the 5′ and 3′ ends respectively. The pBIOCAM vectors were constructed to encode appropriate restriction sites downstream of mammalian leader sequences and upstream of light chain and heavy chain constant sequences as shown in FIG. 9. The construct was created in the backbone of pCMV/myc/ER (InVitrogen, V83220) where genes of interest are driven off a CMV promoter.

In the construct pBIOCAM7-3F, the VL-CL and VH-CH1 chains are cloned in the same vector and are transcribed as a single mRNA with an intervening P2A sequence which causes ribosome stalling during translation. (De Felipe & Ryan (2004) “Targeting of proteins derived from self-processing polyproteins containing multiple signal sequences” Traffic 5, pages 616-626).

The P2A sequence (GSGATNFSLLKQAGDVEENP) is fused between the light chain and the leader sequence of the heavy chain. This would result in the addition of 19 of these 20 amino acids (excluding the terminal proline) being appended to the C terminus of the CL domain. To avoid this, a furin protease site (RAKR) was inserted between the CL and the P2A sequence. It is anticipated that Furin cleavage will remove the P2A sequence leaving the sequence SRRAKR after the terminal cysteine at the C terminus of the light chain product. A similar approach has previously been described (Camper et al (2011) “Stable expression and purification of a functional processed Fab′ fragment from a single nascent polypeptide in CHO cells expressing the mCAT-1 retroviral receptor”. Journal of Immunological Methods 372: 30-41). As a result, separate light and heavy chains are made from a single promoter and expression levels are closely linked. In pBIOCAM7-3F, a hexahistidine tag and a tri-FLAG tag are appended to the C-terminus of the heavy chain. As an alternative, Fabs can be produced by separate cloning of VL into pBIOCAM1 and VH into pBIOCAM3. Versions of the vector are available with C terminal his/triFLAG tags (designated “−3F”) or without tags.

The VHs and VLs from all 8 lead antibodies were cloned into the appropriate sites of pBIOCAM7-3F. Transient transfection of human embryonic kidney cells (HEK293 cells) was conducted using standard methods (Tom et al, 2007; Backiwal et al, 2008; Wulhfard et al, 2010). Following transfection, the HEK293 cells were grown in Gibco Freestyle media with 1% serum added for five days before harvesting the expressed Fab protein. The Fab protein was purified using IMAC on Ni-NTA resin (QIAGEN).

### EXAMPLE 7

### Characterisation of 7A2 and Improved Variants

### Affinity Measurement

The binding characteristics of 7A2 and its derivatives were measured using a GE Healthcare Biacore biosensor. The MET-928 extracellular domain was coupled to a Biacore biosensor surface using standard amine coupling (following the manufacturer's recommendations). Samples of the purified antibody were then passed over the MET derivatised surface and the affinity of the 7A2 scFv antibody was found to be approximately 12 nM (Table 12), and the 7A2 derivatives all displayed a significantly improved affinity for MET.

TABLE 12
Affinities of 7A2 and variants for MET-928

| Antibodies in single chain format | | | | Antibodies in Fab format | | |
|---|---|---|---|---|---|---|
| Clone | ka1 (1/Ms) | Kd1 (1/s) | KD1 (M) | ka1 (1/Ms) | kd1 (1/s) | KD1 (M) |
| 107_A01 | 4.92E+07 | 0.003208 | 6.52E−11 | 8.63E+05 | 0.001233 | 1.43E−09 |
| 107_A07 | 3.31E+06 | 0.001146 | 3.46E−10 | 9.49E+05 | 0.001164 | 1.23E−09 |
| 107_A08 | 2.52E+07 | 0.006909 | 2.74E−10 | 8.38E+05 | 0.001915 | 2.29E−09 |
| 107_G06 | 1.37E+06 | 8.86E−04 | 6.46E−10 | | | |
| 110_A01 | 5.05E+06 | 0.001626 | 3.22E−10 | 1.12E+06 | 0.001372 | 1.23E−09 |
| 110_A10 | 9.08E+06 | 0.002727 | 3.01E−10 | 7.56E+05 | 0.001682 | 2.23E−09 |
| 110_A12 | 1.63E+06 | 0.001557 | 9.58E−10 | 5.77E+05 | 0.001612 | 2.79E−09 |
| 110_A06 | 3.45E+07 | 0.004166 | 1.21E−10 | 6.77E+05 | 0.001284 | 1.90E−09 |
| 111_D06 | 3.01E+07 | 0.005488 | 1.83E−10 | 8.60E+05 | 0.001971 | 2.29E−09 |
| 112_G01 | 3.03E+06 | 0.00238 | 7.86E−10 | | | |
| 7A2 | 5.25E+07 | 0.6463 | 1.23E−08 | 1.14E+06 | 0.08815 | 7.73E−08 |

### Cell Migration Study of 8 Affinity Matured Variants of 7A2

The affinity data relating to the His-tagged, affinity matured variants which were successfully converted into Fab format are detailed above. Two additional clones which were only available in the scFv format are also included. These variants were analysed for inhibition of HGF/SF-induced cell migration using a modified Boyden chamber assay (AC96 Migration Chamber; Neuroprobe). Lower chambers containing 30 pM HGF/SF and antibodies diluted in Assay Media (a 1:1 mixture of PBS and RPMI, 0.25% BSA) were separated from upper chambers by a porous membrane (8 μm, PVP-free) that had been coated with 100 μg/ml Collagen (Purecol, Nutacon) for 2-3 hours at room temperature. SKOV-3 cells were labelled with the fluorescent dye Calcein AM (Life Technologies) at a concentration of 5 μM for 30 minutes at 37° C. prior to washing and resuspension in Assay Media; 50,000 cells were then added to each upper well and the chamber incubated at 37° C. for 4 hours to allow cell migration to occur. The apparatus was disassembled and non-migrated cells removed from the membrane by gentle wiping with cotton wool. The degree of cell migration was assessed by quantification of the residual fluorescence, indicative of migrated cells, on a Typhoon instrument (GE Life Sciences), using excitation/emission settings of 488 nm/526 nm respectively. Data were analysed with ImageQuant software and background fluorescence subtracted.

FIG. 10 shows that all eight of the affinity matured Fabs inhibited HGF/SF-induced cell migration with significantly higher potency than parental 7A2 Fab. Mean IC50s were calculated for clones 7A2, 107_A07 and 110A01 (Table 13), giving 386 nM, 7.4 nM and 13.1 nM respectively.

TABLE 13
Inhibition of HGF/SF-induced migration of SKOV-3
cells by selected anti-Met Fabs: IC50 estimations. GraphPad
Prism software was used to fit sigmoidal dose-response curves.
Curve ends were constrained to migration values in the absence
of antibody (top) and the absence of HGF/SF (bottom) for each assay.

| | Assay 1 | Assay 2 | Assay 3 | Mean IC50 |
|---|---|---|---|---|
| 7A2 | 386.6 | 493.4 | 278.5 | 386 |
| 107_A01 | 9.7 | | 11.4 | 10.6 |
| 107_A07 | 9.7 | 9.2 | 3.1 | 7.4 |
| 107_A08 | 10.5 | | | 10.5 |
| 110_A01 | 9.6 | 27.0 | 2.6 | 13.1 |

FIG. 10 shows that migration of fluorescently-labelled SKOV-3 human ovarian cancer cells towards 30 pM HGF/SF is inhibited in the presence of anti-Met Fabs 7A2, 107_A01, 107_A07, 107_A08, 110_A01, 110_A06, 110_A10, 110_A12 and 111_D06.

Characterisation of Monomeric 7A2 scFv

Since scFv can multimerise, possibly complicating analysis, we also analysed the biological activity of monomeric 7A2 scFv. 8×His-tagged 7A2 scFv (Table 14) was expressed in P. pastoris induced with Methanol over 3 days. Conditioned media was concentrated using a Sartorius Slice Disposable (10 kD nominal molecular weight cut-off) and the scFv purified on a HisTrap column (GE Life Sciences). Monomeric 7A2 scFv (FIG. 11) was isolated by size exclusion using a Superdex 200 16/60 prep grade column (GE Life Sciences). FIG. 10 shows that monomeric 7A2 scFv inhibits HGF/SF-induced migration of SKOV-3 cells with 50% inhibition at 15 nM. Biacore analysis of monomeric 7A2 scFv binding to a Met928-coated surface gave a KD of 3.6 nM (Table 15).

TABLE 14
Amino Acid sequence of 7A2 scFv, as
expressed in P. pastoris. The first
two amino acids, or the first four
amino acids, may be cleaved during
processing.

7A2 sequence
EAEAQMQLVQSGAEVKKPGAPVKVSCKVSGYTFTDYYMHWVQQA
PGKGLEWMGLVDPEDGETIYAEKFQGRVTITADTSTDTAYMELS
SLRSEDTAVYYCATDATTPYYGMDVWGQGTLVTVSSLEGGGGSG
GGGSGGGASDIQMTQSPSSLSASVGDRVTITCQASQDISNYLNW
YQQKPGRAPKVLIYDASNLETGVPSRFSGSGSGTEFTLTISNLR
PDDFATYYCQQGDSFPLTFGGGTKVEIKRPRHHHHHHHH

Excluding the first four amino acids, and excluding the last 11 amino acids (the HIS tag), this is the 7A2 scFv sequence of SEQ ID No: 26.

TABLE 15
Kinetic constants for isolated monomeric 7A2
scFv exposed to a MET928-coated Biacore surface

| Ka (1/Ms) | Kd (1/s) | KD (M) |
|---|---|---|
| 5.858 × 10⁵ | 0.002121 | 3.620 × 10⁻⁹ |

### Affinity Characterisation of Tagless 7A2 Fab

For further studies, 7A2 Fab was expressed without His or FLAG tags (Table 16).

Following transient transfection of HEK293F cells with plasmids encoding both heavy (pBIOCAM1) and light (pBIOCAM3) chains, 7A2 Fab was purified using a KappaSelect column (GE Life Sciences). 7A2 Fab produced and purified in this manner behaved similarly to His- and FLAG-tagged 7A2 Fab in cell migration assay (FIG. 10) and in Biacore analysis (Table 17).

TABLE 16
Amino Acid sequence of heavy and
light chains of 7A2 Fab (tagless format).

7A2 Fab heavy chain amino acid sequence
(SEQ ID No: 28)
QMQLVQSGAE VKKPGAPVKV SCKVSGYTFT DYYMHWVQQA
PGKGLEWMGL VDPEDGETIY AEKFQGRVTI TADTSTDTAY
MELSSLRSED TAVYYCATDA TTPYYGMDVW GQGTLVTVSS
LEASTKGPSV FPLAPSSKST SGGTAALGCL VKDYFPEPVT
VSWNSGALTS GVHTFPAVLQ SSGLYSLSSV VTVPSSSLGT
QTYICNVNHK PSNTKVDKKV EPKSCLQGGG S
7A2 Fab light chain amino acid sequence
(SEQ ID No: 29)
ASDIQMTQS PSSLSASVGD RVTITCQASQ DISNYLNWYQ
QKPGRAPKVL IYDASNLETG VPSRFSGSGS GTEFTLTISN
LRPDDFATYY CQQGDSFPLT FGGGTKVEIK RAAATVAAPS
VFIFPPSDEQ LKSGTASVVC LLNNFYPREA KVQWKVDNAL
QSGNSQESVT EQDSKDSTYS LSSTLTLSKA DYEKHKLYAC
EVTHQGLSSP VTKSFNRGEC SRASGS

TABLE 17
Kinetic constants for 7A2 Fab expressed in a
tagless format and purified on KappaSelect resin.

| Ka (1/Ms) | Kd (1/s) | KD (M) |
|---|---|---|
| 5.967 × 10⁵ | 0.1079 | 1.808 × 10⁻⁷ |

Based on the above data, clone 107A07 was selected for further analysis and was also cloned into a tagless format (Table 18).

TABLE 18
Amino acid sequence of the Fab heavy and light
chains of the tagless version of antibody 107-A07.

107-A07 Fab heavy chain amino acid sequence
(SEQ ID No: 30)
QMQLVQSGAEVKKPGAPVKVSCKVSGYTFTDYYMHWVQQAPGK
GLEWMGLVDPEDGETIYAEKFQGRVTITADTSTDTAYMELSSL
RSEDTAVYYCATDATTPYWGMMWWGQGTLVTVSSASTKGPSVF
PLAPSSKSTSGGTAALGCLVKDYFPEPVTVSWNSGALTSGVHT
FPAVLQSSGLYSLSSVVTVPSSSLGTQTYICNVNHKPSNTKVD
KKVEPKSC
107-A07 Fab light chain amino acid sequence
(SEQ ID No: 31)
DIQMTQSPSSLSASVGDRVTITCQASQDISNYLNWYQQKPGRA
PKVLIYDASNLETGVPSRFSGSGSGTEFTLTISNLRPDDFATY
YCQQGDSFPLTFGGGTKVEIKRTVAAPSVFIFPPSDEQLKSGT
ASVVCLLNNFYPREAKVQWKVDNALQSGNSQESVTEQDSKDST
YSLSSTLTLSKADYEKHKLYACEVTHQGLSSPVTKSFNRGEC

The extra amino acids inserted at the end of the heavy chain variable region and the light chain variable region to create cloning sites for the construction of the original human antibody library (Schofield et al. 2007) have been removed in order to restore the natural amino acid sequence found in human antibodies.

### EXAMPLE 8

### Epitope Mapping

### 7A2 and Affinity-Matured Variant 107_A07 Recognise an Epitope Located on Met741 and MET928, but not on MET567

7A2 and 107_A07 were expressed as tagless Fabs (Tables 10 and 12) by PEI-mediated transient transfection of HEK293F cells with plasmids encoding the VH-CH (heavy chain) and VL-CL (light chain). Fabs were purified on KappaSelect resin (GE Life Sciences). 5D5 Fab was purified first by IMAC and, prior to this experiment, by gel filtration on a Superdex 200 10/300 column.

Epitope location was analysed by analytical size exclusion chromatography. Met extracellular domain of differing lengths (MET567=aa 25-567, MET741=aa 25-741, MET928=aa 25-928) was incubated with a 1.5-fold molar excess of Fab for a minimum of 2 hours at room temperature, in 25 mM Tris pH7.4, 150 mM sodium chloride. The mixtures were then centrifuged to remove any debris and analysed on a Superdex 200 10/300 size exclusion column (GE Life Sciences). 7A2 and 107_A07 can both bind to the MET741 fragment but not to the MET567 fragment indicating that the epitope lies within the Met 568-741 region of the protein which contains the IPT1 and IPT2 domains. Although both antibodies can also bind to MET928, stoichiometric analysis based on the 1.5-fold molar excess of antibody used in the binding experiment with MET928 and the fact that a clear unbound fraction remains suggests that there is not an additional epitope binding site for the antibodies within the 742-928 region of the protein (covering the IPT3 and 4 regions).

FIG. 12 shows that incubation of either 7A2 or 107_A07 with MET741 resulted in the appearance of a species that migrated more slowly (indicating a larger molecular size) than either MET or Fab alone. Similarly, incubation of 7A2 or 107_A07 with MET928 resulted in the appearance of a species migrating slower than MET928 or Fab alone. The appearance of this new peak, combined with the disappearance of the Met peak and the reduction in the magnitude of the Fab peak, indicates that the MET and the Fab have formed a complex. In contrast, when MET567 was incubated with either 7A2 or 107_A07, no additional peak was observed and the MET and Fab peaks remained in their original locations. Thus, neither 7A2 nor 107_A07 form a complex with MET567, indicating that the binding site for 7A2 and 107_A07 is located on MET741 and MET928, but not on MET567.

In contrast, when 5D5 was incubated with MET567 under the same conditions, a new peak was observed with larger molecular size than MET567 or 5D5 alone. This indicates that, unlike 7A2 and 107_A07, 5D5 forms complexes with MET567, indicating that the binding site for 5D5 is located on MET567.

Generally the “hinged” (and tagged) format was used for all screening experiments (including side-by-side comparison of affinity matured variants and selection of 107_A07 as the lead clone); 107_A07 was then converted to the “native” (i.e., not “hinged”) sequence, and the tags removed, for full characterisation. Surprisingly, FIG. 15 shows that the “hinged” (and also tagged, and IMAC-purified) 107_A07 Fab displays an even greater degree of antagonism at very high (micromolar) concentrations compared to the “unhinged” Fab. Thus, it may be possible to improve the level of antagonism at high concentrations of all of the Fab antibodies by the inclusion of a “hinge” region between the heavy and light chains.

7A2 and 5D5 Bind Distinct Epitopes on cMet

We used Surface Plasmon Resonance to analyse comparative binding of 5D5 and 7A2 to cMet. The MET928 extracellular domain was coupled to a Biacore CM5 biosensor surface using standard amine coupling (following the manufacturer's recommendations). His-tagged 7A2 scFv was produced in P. pastoris and purified on HisTrap resin (GE Life Sciences). A further purification on a Superdex 200 16/60 prep grade column allowed isolation of purely monomeric scFv. His-tagged 5D5 was expressed in Fab format and purified by IMAC. Preliminary kinetic studies identified concentrations of each antibody that, in isolation, caused near-saturation binding to the chip: 134 nM 7A2, and separately, 125 nM 5D5.

FIG. 13 shows that shows that 125 nM 5D5 (lower thin line) approaches saturation of the 5D5 epitope, as 250 nM 5D5 (upper thin line) causes relatively little increase in signal. Similarly, 134 nM 7A2 (lower medium-thickness line) approaches saturation of the 7A2 epitope, as doubling that concentration (268 nM 7A2, upper medium-thickness line) causes a relatively small increase in binding. However, exposing the chip to a mixture containing 134 nM 7A2 and 125 nM 5D5 leads to a much larger increase in overall binding. Thus, 5D5 can still bind well to Met when the 7A2 epitope is approaching saturation, and 7A2 can bind well to Met when the 5D5 epitope is approaching saturation. Together with the size exclusion chromatography data above, these data confirm that the 7A2 and 5D5 epitopes are distinct.

### Further Characterisation of 107_A07

7A2, 107_A07 and the negative control anti-lysozyme antibody D1.3 (Foote & Winter, 1992, J. Mol. Biol. 224: 487-499) in tagless Fab format were further purified using the GammaBind Plus resin (GE Life Sciences), which recognises antibody Gamma chains. Purified 7A2, 107_A07, D1.3 and 5D5 Fabs were dialysed into PBS (pH7.4) and quantitated by applying sequence-specific extinction coefficients to the absorbance at 280 nm. FIG. 14, part 2, shows a Coomassie-stained SDS-PAGE gel in which equal amounts (1 μg) of each Fab were loaded for comparison.

TABLE 19
Amino acid sequence of the Fab heavy and light
chains of the tagless version of antibody D1.3.

D1.3 Fab heavy chain amino acid sequence
QVQLQESGPGLVRPSQTLSLTCTVSGSTFSGYGVNWVRQPPGRGL
EWIGMIWGDGNTDYNSALKSRVTMLVDTSKNQFSLRLSSVTAADT
AVYYCARERDYRLDYWGQGSLVTVSSASTKGPSVFPLAPSSKSTS
GGTAALGCLVKDYFPEPVTVSWNSGALTSGVHTFPAVLQSSGLYS
LSSVVTVPSSSLGTQTYICNVNHKPSNTKVDKKVEPKSC
D1.3 Fab light chain amino acid sequence
DIQMTQSPSSLSASVGDRVTITCRASGNIHNYLAWYQQKPGKAPK
LLIYYTTTLADGVPSRFSGSGSGTDFTFTISSLQPEDIATYYCQH
FWSTPRTFGQGTKVEIKRTVAAPSVFIFPPSDEQLKSGTASWCLL
NNFYPREAKVQWKVDNALQSGNSQESVTEQDSKDSTYSLSSTLTL
SKADYEKHKLYACEVTHQGLSSPVTKSFNRGEC

With the exception of FIG. 15, All the data in this section (SPR, migration, DNA synthesis) used tagless and “hinged” 7A2, tagless “native” (i.e., not hinged) 107_A07 (purified as described above), and tagged 5D5 purified by IMAC. The SDS-PAGE data shows that all these preparations were highly pure. In FIG. 15, “hinged” His-tagged, IMAC-purified Fabs were used.

The affinity of 107_A07 for Met was measured by Surface Plasmon Resonance. 1:1 kinetic analysis of the highly pure Fabs binding to a MET928-coated surface gave a KD of 4.2 nM for 107_A07 and 6.9 nM for 5D5 (Table 20)

TABLE 20
Biacore analysis of 107_A07 and
5D5 binding to a MET928-coated surface.

| | Ka (1/Ms) | Kd (1/s) | KD (M) |
|---|---|---|---|
| 107_A07 Fab | 1.8 × 10⁶ | 0.0075 | 4.2 × 10⁻⁹ |
| 5D5 Fab | 1.2 × 10⁵ | 8.1 × 10⁻⁴ | 6.9 × 10⁻⁹ |

7A2, 107_A07 and 5D5 Fabs were analysed for inhibition of HGF/SF-induced migration of SKOV-3 cells. FIG. 14 shows that 7A2 and 107_A07 significantly inhibit HGF/SF-induced cell migration. Notably, migration in the presence of 107_A07 at lower concentrations (for example, 5-150 nM) was lower than that in the presence of 5D5.

TABLE 21
Fab concentrations (nanomolar) giving 50% inhibition of HGF/SF-
induced SKOV-3 migration in the modified Boyden chamber assay.

| | MEDIAN | MEAN | SD | n |
|---|---|---|---|---|
| 107_A07 | 29 | 29 | 18 | 5 |
| 5D5 | 91 | 101 | 29 | 5 |

n = number of independent determinations;
SD = standard deviation.

In the absence of HGF/SF, at concentrations of up to 200 nM 107_A07 FAB, stimulation of cell migration (which would be expected by an agonist) was not typically observed. Trace agonism (Mean±standard deviation of six assays 1.8±0.5 fold background, approximately 5% of HGF/SF-dependent migration at 30 pM HGF/SF) was observed at 1000 nM and may be due to protein aggregation (in another assay using size-exclusion purified 107_A07, agonism was not observed at any concentration including 1000 nM, data not shown).

FIG. 16 shows that 107_A07 and 7A2 significantly inhibit HGF/SF-dependent proliferation of BxPC3 cells, assessed by a BrdU incorporation assay.

### Mechanism of Action

FIG. 18A demonstrates that binding of 7A2, 107_A07 and 5D5 Fabs to the Met extracellular domain is inhibited by HGF/SF, indicating that all three Fabs compete at least partially with HGF/SF for binding to the Met extracellular domain. FIG. 17 shows that, apparently in contrast to 5D5 Fab, 7A2 scFv competes with NK1 (the major to receptor-binding region of HGF/SF) for binding to MET extracellular domain. Similarly, in a competition ELISA (FIG. 18B), binding of 2.5 nM 7A2 Fab to a MET928-coated surface is inhibited by 15 μM and 5 μM NK1. Thus, NK1 and 7A2 compete for binding to MET. Binding of 107_A07 to a MET928-coated surface is also inhibited by 15 μM and 5 μM NK1, in contrast to 5D5, which is not inhibited by NK1 at these concentrations (FIG. 18B). To our knowledge this is the first report of an antibody that can compete with the NK1 fragment of HGF for binding to MET.

TABLE 22
Amino Acid sequence of the NK1 fragment
of HGF/SF. NK1 was produced in Pichia
pastoris. The first two amino acids (EA),
or the first four amino acids (EAEA),
may be removed during processing.

Amino acid sequence of the NK1 fragment
used in these studies
(SEQ ID NO: 27)
EAEAYVE GQRKRRNTIH EFKKSAKTTL IKIDPALKIK
TKKVNTADQC ADRCTRNKGL PFTCKAFVFD KARKQCLWFP
FNSMSSGVKK EFGHEFDLYE NKDYIRNCII GKGRSYKGTV
SITKSGIKCQ PWSSMIPHEH SFLPSSYRGK DLQENYCRNP
RGEEGGPWCF TSNPEVRYEV CDIPQCSEVE

### Further Modifications Affecting Efficacy

We compared 7A2 Fab in a native-like sequence to 7A2 Fab with an additional two amino acids (Leu-Glu) inserted at the C terminus of the heavy chain variable region. Surprisingly, the addition of these extra amino acids improved the efficacy of the Fab at inhibiting HGF/SF-dependent SKOV-3 cell migration (FIG. 19). Without wishing to be bound by any theory, we consider that one explanation could be that the constant domains are inhibiting the variable domains of the antibodies, and that addition of the extra amino acids reduces that inhibition, for example by increasing flexibility between the variable and constant domains. We expect that the efficacy of 107_A07 and other Fabs might similarly be improved by addition of these extra amino acids.

### EXAMPLE 9

### Co-Crystallisation of Met/Fab Complex

### Complex Formation and Purification

In order to define the epitope recognised by 7A2/107_A07 to a high degree of resolution, crystals of the complex were formed and the structure of the complex determined by X-ray crystallography. Crystallisation was assisted by deglycosylation and proteolytic digestion of the complex with pepsin to yield a fragment containing at minimum amino acids 519-740 of c-Met. MET741 with a C terminal hexahistidine tag and untagged 107_A07 FAb were co-incubated for 140 minutes at a 1:1.6 molar ratio prior to addition of EndoHf (New England Biolabs) and Pepsin (Sigma Aldrich P7012) before a further incubation for 48 hours on ice, to allow MET deglycosylation and cleavage of the SEMA domain. Each enzyme was used at a MET741:enzyme ratio of 10:1 (w/w). Following digestion, the processed complex was purified by gentle mixing for one hour at 4° C. with NiNTA-agarose (Qiagen). The mixture was transferred to a Proteus Midi Spin Column (Generon, UK) and washed twice with 10 ml 100 mM NaH2PO4 pH8.0, 300 mM NaCl, 20 mM Imidazole, prior to elution with 10 ml NaH2PO4 pH8.0, 300 mM NaCl, 200 mM EDTA. Cleaved, digested MET-FAb complex was further purified and buffer-exchanged into crystallisation buffer (25 mM Tris pH7.4, 200 mM sodium chloride, 7.5% v/v glycerol) by gel filtration with a Superdex 200 16/60 column (GE Life Sciences). Prior to gel filtration the mixture was exposed briefly to Dextrin-Sepharose (GE Life Sciences) as a precaution against contaminating EndoHf, which elutes at a similar volume to the digested MET-FAb complex but due to fusion with Maltose Binding Protein is expected to bind Dextrin-Sepharose. Purified complex eluted essentially as a single peak and was concentrated to approximately 5.9 mg/ml with a 10 kD MWCO Amicon Ultra-15 centrifugal filter device (Millipore).

Analysis of the purified protein complex by non-reducing polyacrylamide gel electrophoresis and InstantBlue protein staining (Expedeon Ltd., Harston, UK) showed two major protein bands, consistent with intact 107_A07 FAb (upper band) and a proteolytic fragment of hexahistidine tagged MET741 migrating at approximately 32 kD.

### Protein Crystallization

Sitting-drop vapour diffusion crystallisation trials (total volume 400 nl drops, 1:1 ratio of protein:precipitant) were set up in MRC 2-drop 96-well crystallisation plates using the Phoenix crystallization robot (Art Robbins Instruments, Inc.). The crystallization trials were incubated at 19° C. and monitored in ROCK Imager 500 (Formulatix Inc) automated imaging system. Following the initial crystallization hits with the Morpheus protein crystallization screen (Molecular Dimensions), 1 μl+1 μl drops were manually set up with Morpheus condition A9 (10% PEG 20,000, 20% PEG 550-MME, 0.1 M Trizma/Bicine pH 8.5, 0.03M Magnesium Chloride, 0.03M Calcium Chloride) in a 24-well Intelliplate (Art Robbins Instruments, Sunnyvale, Calif., USA). Prior to contact with the protein, the precipitant was supplemented with 5% (v/v) glycerol. Glycerol was not added to the reservoir. Crystals appeared within 24 hours and were harvested on day seven, incubating briefly for cryoprotection in Morpheus condition A9 supplemented with 20% glycerol prior to freezing in liquid nitrogen.

Determination of Structure of Complex of 107_A07 and c-Met

The X-ray data collection experiments were performed at 100K temperatures at the European Synchrotron Radiation Facility (ESRF, Grenoble France), beamline ID29. The crystals diffracted to a maximum resolution of 2.6 Å (the resolution cut-off level was set to the resolution shell were the average I/sigma of the reflections is still greater than 2). A total of 150 degrees of data were collected at 0.05 degree oscillation angle. The crystals belonged to the P22₁2₁ space group and contained two molecules of the MET/FAb complex (approximate molecular weight of the complex 71.3 kDa) in the asymmetric unit. This results in approximately 56% crystal's solvent content (Matthews Coefficient of 2.77). All diffraction data were indexed, scaled and merged using XDS Suite (Kabsch, W. XDS. Acta Cryst. D66, 125-132 (2010)), the crystallographic data collection statistics are shown in Table 23.

TABLE 23
Crystallographic data collection and refinement statistics

| | MET/FAb complex |
|---|---|
| Data collection | |
| Radiation Source, Beamline | ESRF, ID29 |
| Wavelength (Å) | 0.91376 |
| Space group | P22₁2₁ |
| Cell dimensions: | |
| a, b, c (Å) | 71.89, 82.29, 267.33 |
| α, β, γ (°) | 90.0, 90, 90 |
| Resolution (Å) | 48.95 − 2.60 (2.74 − 2.60)¹ |
| R_{cryst} | 11.1 (88.3) |
| </ / σ(/)> | 11.7 (1.9) |
| Completeness (%) | 99.0 (95.8) |
| Redundancy | 5.6 (5.5) |
| No. of unique reflections | 49,099 (6,840) |
| Refinement | |
| Resolution (Å) | 48.9 − 2.60 |
| No. of reflections: | |
| Total | 48,932 |
| R_{free} set | 1,999 |
| R_{work}/R_{free} | 23.1/27.8 |
| Contents of asymmetric unit: | |
| Protein atoms | 9798 |
| Solvent atoms | 21 |
| R.m.s deviations: | |
| Bond lengths (Å) | 0.003 |
| Bond angles (°) | 0.732 |

¹The statistics shown in parentheses are for the highest-resolution shell.

The crystal structure of the MET/FAb complex was solved using the Molecular Replacement (MR) method. The positions of individual domains, i.e. the constant and variable domains of the heavy and the light chains of Fab and the two immunoglobulin (Ig) domains of the MET receptor fragment within the asymmetric part of the unit cell were identified. All MR calculations were performed in PHASER (part of PHENIX software suite distribution, version 1.7.2-869).

The MR search probes included the constant and variable domains of the heavy and the light chains of Fab fragment crystal structure (PDB-ID: 1RZ7) and the two Ig domains from the crystal structure of MET in complex with the Listeria invasion protein InIB (PDB-ID: 2UZY). All positional solutions of the domains (even for the first domain) had the translation function Z-score values above 8, indicating the correctness of the MR solution. The refinement calculations were performed in PHENIX while manual rebuilding was performed in COOT. The first round of refinement calculations with the obtained model caused a significant drop in R/R_{free} values by about 6% each, reaching the final values of 33.4% and 37.4%, respectively. Thus indicating the correctness of the obtained MR solution. After 4 rounds of manual rebuilding and refinement the in R/R_{free} values are 23.1% and 27.8%, respectively.

The structure of the complex is shown in FIGS. 21A and 21B.

Analysis of the Interaction Between 107_A07 and c-Met

The sequence of the antibody FAb fragment 107_A07 within the structure is as shown in Table 18 heavy chain (SEQ ID No 30) and light chain (SEQ ID 31). In all cases an additional Ala Ser residues is present at the N terminus of the antibodies and comes from the Nhe1 site used to clone the antibody VL downstream of the mammalian leader sequence in pBIOCAM vectors.

Following identification of the Met Ig1 and Ig2 domains within the crystal structure by Molecular Replacement, Met residues 519-740 could be resolved. The sequence of MET515-741 is shown in FIG. 22A and the comparison of mouse and human Met around the binding site is shown in FIG. 22B. The structure of the complex reveals the critical residues that define the epitope of the antibody, resulting in a blockade of HGF/SF signaling through Met. These are R592, N593, K595, K599, K600, R602, T611, T613, L614, S615.

A surface representation of 107_A07 showing residues in contact with Met is given in FIG. 23. Important contact residues of the heavy chain are residues D31, Y33, D52, D55, E57, D99, A100, T101, T102, P103, Y104, W105. For the light chain residues Y32 and D92 are involved in direct contact with Met. The antibody 107_A07 used for the structural determination was derived from the parental clone 7A2 by changing only 3 amino acids present within CDR3 of the heavy chain. The sequence of the parental clone 7A2 (attpyygmdv) was converted to attpyWgmMW (changes shown in upper case). These changes (Y105W, D108M, V109W) had a significant effect on the affinity and potency of the resultant clones. From the structure it is not clear however why those would produce stronger binders. Y105 is making contact but it is a purely hydrophobic contact, and this particular mutation may be more important in the interaction with the light chain rather than the Met. The other two are not making any contacts with Met but those mutations could play indirect role by potentially stabilising the position of CDR3. An alternative explanation is that these residues are important in making contact with other residues within MET928, which are not present in the MET515-714 used for the structural determination.

Examination of the structure suggests that only 2 residues of the light chain (Y32 and D92) are making contact with MET515-574. It is possible that other residues in the light chain make contact with residues 1-514, which include the β propeller domain of Met which are absent in this complex.

### EXAMPLE 10

### Inhibition of Tumour Growth in Mouse Xenografts by Polyethylene Glycol Modified 107_A07 Fab Fragments

### Expression and Purification of Fab Antibody Preparations

In order to assess the ability of 107_A07 to affect tumour growth in mouse xenograft experiments, a FAb formatted antibody preparation of 107_A07 was compared with a negative control Fab formatted antibody (humanized anti-lysozyme D1.3 (huD1,3). Cloning of and expression of Fab formatted 107_A07 and huD1.3 in pBIOCAM1 and pBIOCAM3, transient transfection into in HEK293 cells and antibody purification were as described in Example 8 and original patent FIG. 9. In short FAbs were produced by PEI-mediated transient transfection of HEK293F cells (Invitrogen) grown in suspension in FreeStyle293 media. Valproic Acid (Sigma) was added to 4 mM following transfection and conditioned media collected after approximately one week. FAbs were purified using affinity resins KappaSelect and/or GammaBind Plus (GE Life Sciences).

It is known that Fab formatted antibodies have short half-lives in vivo. Modification of antibodies and other proteins by addition of polyethylene glycol is known to increase the circulating half-life of such molecules. The extended half-life of such PEG modified molecules will increase the residence time in serum of these antibodies with potential benefits in potency.

A protocol was developed for crosslinking maleimide activated PEG molecules to the Fab formatted antibodies. The Fab antibody is first treated with TCEP (tris(2-carboxyethyl)phosphine), a reducing agent, which breaks the disulphide bond formed by the terminal cysteines on the light and heavy chains. Maleimide group reacts specifically with sulfhydryl groups to form a covalent thioether linkage when the pH of the reaction mixture is low. The reaction between TCEP modified FAb and maleimide PEG was carried out at pH 6.0 (Humphreys et al (2007) Protein Engineering, Design & Selection vol. 20 pp. 227-234). A maleimide activated branched 20 kd PEG molecule was used for antibody modification (from NOF EUROPE, 20,000 mw maleimide activated PEG-C2 type Catalogue No: Sunbright ME-200MA).

Fab antibody was dialysed into PEGylation buffer (100 mM phosphate buffer with 2 mM EDTA. pH6, Humphreys et al, 2007) and 1/10^{th} volume of 50 mM TCEP was added (Pierce cat no. 20490, prepared in water and pH adjusted to pH 6-6.5). Incubation was carried out for 30 minutes at room temperature. It is important to remove TCEP before PEGylation of the antibody and incomplete removal of TCEP will greatly reduce the efficiency of PEGylation. TCEP does not behave as expected for a small molecule during gel filtration, eluting much closer to the protein peak than expected (Shafer et al (2000) Analytical Biochemistry 282, 161-164). TCEP was removed by spinning through a Zeba column (Pierce) pre-equilibrated with PEGylation buffer. For a 2 ml Zeba column add a maximum of 500 ul of protein and for a 10 ml Zeba column a maximum of 2 ml Maleimide activated PEG (stored at −70° C.) was equilibrated to room temperature and dissolved in PEGylation buffer immediately before use to a concentration of 20 mg/ml. For the attachment of two 20 KDa peg molecules to a 40 KDa Fab antibody we used 2.5 mg of PEG per mg of antibody (Humphreys et al, 2007). The reaction was incubated at room temperature with gentle end over end mixing for 2 hours and then left stationary overnight.

After PEG coupling the sample was processed through a 30 kD centrifugal filter device (Amicon Ultra-15, 30 kD, UFC903024, regenerated cellulose membrane) to remove some of the free PEG. The PEGylated Fab was further separated from PEG and non-pegylated Fab by gel filtration using several runs on a Superdex 200 column. Samples were then concentrated using an Amicon Ultra-15 centrifugal filter unit (UFC901024) spun at 3700-4000 g.

### Effect of 107_A07-PEG on HGF/SF-Induced Cell Migration

Biological activity of 107_A07-PEG was confirmed by cell migration assay, performed as described in previous sections. PEGylated 107_A07 was able to completely block HGF/SF-induced migration of SKOV3 cells (FIG. 24).

### Effect of 107A07-PEG on Tumour Growth In Vivo

10⁷ U87MG human glioblastomas cells were sub-cutaneously injected into nude mice NMRI nu/nu nude mice. Mice were placed in groups of 10 and dosed with PEGylated FAb or vehicle alone on days 12, 15, 19 and 22 following tumour implantation on day 0. Three different doses of antibody were tested (1, 3, 10 mg/kg). Tumour sizes were measured by caliper twice weekly and recorded. Individual tumor volumes (TV) were calculated according to V=(length×(width)²)/2. FIG. 25 shows a significant reduction in tumour growth in the mice treated with all 3 doses of test antibody (PEG modified 107_A07 Fab) compared with mice treated with PBS or control antibody (PEG modified huD1.3 Fab). This demonstrates the ability of monovalent, PEG modified 107_A07 to reduce tumour growth in U87 xenografts.

### EXAMPLE 11

### Inhibition of Tumour Growth in Mouse Xenografts by Bivalent IgG2 Formatted 107_A07

107_A07 was prepared in an IgG2 format for testing in mouse xenograft studies. The VH fragment of 107_A07 was cloned into the mammalian expression vector pBIOCAM2-IgG2. This is similar to pBIOCAM3 (Example 6, FIG. 9) except the VH is fused to a full length human IgG2 heavy chain in pBIOCAM2-IgG2 (compared with the VH-CH1 domain only in pBIOCAM3 as defined in SEQ ID no:30). The heavy chain variable domain of 107_A07 was cloned into the Nco1/Xho1 sites of pBIOCAM2-IgG2 to create a mammalian expression vector which expresses an antibody heavy chain with the sequence shown in FIG. 26 (SEQ ID No: 32):

QMQLVQSGAEVKKPGAPVKVSCKVSGYTFTDYYMHWVQQA
PGKGLEWMGLVDPEDGETIYAEKFQGRVTITADTSTDTAY
MELSSLRSEDTAVYYCATDATTPYWGMMWWGQGTLVTVSS
ASTKGPSVFPLAPCSRSTSESTAALGCLVKDYFPEPVTVS
WNSGALTSGVHTFPAVLQSSGLYSLSSVVTVPSSNFGTQT
YTCNVDHKPSNTKVDKTVERKCCVECPPCPAPPVAGPSVF
LFPPKPKDTLMISRTPEVTCVVVDVSHEDPEVQFNWYVDG
VEVHNAKTKPREEQFNSTFRVVSVLTVVHQDWLNGKEYKC
AVSNKGLPAPIEKTISKTKGQPREPQVYTLPPSREEMTKN
QVSLTCLVKGFYPSDIAVEWESNGQPENNYKTTPPMLDSD
GSFFLYSKLTVDKSRWQQGNVFSCSVMHEALHNHYTQKSL
SLSPGK

Co-transfection of this vector with the previously described pBIOCAM1 vector (encoding the light chain of 107_A07 defined by seq ID No:31) resulted in production of a bivalent IgG molecule. IgG antibody was expressed in HEK-293F cells. Supernatants were harvested, buffered in PBS (100 ml 10×PBS added per 900 ml supernatant) and filtered (0.45 μm PVDF). Antibody was then purified from the clarified supernatant using Protein G Sepharose FPLC. 0.2M Glycine pH 2.6 was used for elution and fractions were collected directly into neutralisation buffer (1 M Tris-HCl pH 8.5) to minimise exposure to acidic conditions. Protein containing fractions were pooled and dialysed into PBS. Finally, purified samples were filter sterilised and their concentration was determined using absorbance at 280 nm and a theoretical extinction coefficient.

### In Vivo Testing of 107_A07 in IgG2 Format

Although agonism was seen in cell based assays using bivalent formatted 107_A07, this occurs in low nM concentration, and diminished at higher antibody concentrations. The antibody concentrations used in human therapy and in animal model experiments is typically >30 nM creating the possibility that bivalent IgGs would have an overall antagonistic effect on tumour growth/survival. Sub-cutaneous xenografts of U87 cells were prepared as described in Example 2 above. 7 days after injection of tumour cells, 10 mg/kg 107_A07 in IgG2 format was injected twice weekly for 4 weeks and tumour growth compared with control mice receiving either PBS or a control IgG at 10 mg/kg. Tumour growth in 107_A07 treated mice was significantly lower than tumour growth observed in mice treated with PBS or IgG2 controls (FIG. 27).

### EXAMPLE 12

### Characterisation of 7A2 and 107_A07 as Competing with the NK1 Domains of HGF/SF for Met Binding

We assessed whether 7A2 and 107_A07 competed with NK1 and HGF/SF for binding to Met where the FAb/MET interaction took place in solution. FAb at 5-500 nM (FIG. 8) was mixed with 100 nM MET928.6H (hexahistine tagged MET928) and the mixture incubated at room temperature for one hour, prior to addition of the MET928.6H/FAb mixture to microplates coated with HGF/SF or the fragment NK1 and a further one-hour incubation. Bound MET928.6H was then detected with anti-5xHis (Qiagen) followed by DELFIA® Eu-N1 rabbit anti-mouse-IgG, exposure to DELFIA® enhancement solution and quantitation by time-resolved fluorescence on a Fusion instrument (Perkin Elmer).

Surprisingly, pre-incubation of MET ectodomain with either 7A2 or 107_A07 inhibited MET binding to ligand-coated microplates, suggesting that HGF/SF and the NK1 fragment of HGF/SF both compete with 7A2 and 107_A07 for binding to the MET ectodomain (FIG. 28).

### EXAMPLE 13

### 107_A07 Inhibits HGF/SF-Induced Cell Proliferation

U87MG human glioblastoma cells were plated in full media at 100,000 cells/well in a 12-well tissue culture dish (Costar) and allowed to adhere overnight. Cells were serum-starved by washing three times with serum-free media supplemented with 0.25% BSA (SFM/BSA); the third wash was left on the cells for two days. Cells were then exposed for 24 hours to 300 μM HGF/SF with or without 0.9 μM 107_A07 FAb or 1 μM D1.3 FAb. FAb preparations used in this assay had been purified using affinity resins KappaSelect (GE Life Sciences) and GammaBind Plus (GE Life Sciences) and additionally size exclusion chromatography (Superdex 200 10/300 GL column; GE Life Sciences). Cells were trypsinised, fixed with 70% ethanol in PBS, stained with 40 μg/ml propidium iodide in the presence of 100 μg/ml RNAse and analysed by flow cytometry according to standard procedures. Cell population was gated using a plot of forward scatter vs side scatter and doublet discrimination used to ensure analysis of single cells.

Addition of 107_A07 at the same time as HGF/SF reduced the proportion of cells in G2/M and in S-phase, with a corresponding increase in the proportion of cells in G1 (Table 24; FIG. 29).

TABLE 24
Cell cycle analysis of U87MG cells treated with 300 pM HGF/SF
in the presence and absence of 107_A07 or D1.3 FAb.

| | G1 | | S | | G2/M | |
|---|---|---|---|---|---|---|
| | Mean | SD (n) | Mean | SD (n) | Mean | SD (n) |
| HGF/SF + D1.3 | 64% | 2% (3) | 15% | 2% (3) | 21% | 1% (3) |
| HGF/SF + | 81% | 3% (3) | 9% | 2% (3) | 10% | 0.5% (3) |
| 107_A07 | | | | | | |
| HGF/SF | 64% | 3% (3) | 17% | 2% (3) | 18% | 4% (3) |
| No HGF/SF | 82% | ND (2) | 7% | ND (2) | 11% | ND (2) |

### SELECTED REFERENCES

Backiwal, G. et al (2008) Nucleic Acids Research 36(15): e96
Basilico, C. et al (2008). J. Biol. Chem. 283(30): 21267-77.
Bellon et al, (2008) “c-Met inhibitors with novel binding mode show activity against several hereditary papillary renal cell carcinoma-related mutations.” J Biol Chem. 283(5): 2675-83.
Birchmeier, et al (2003). Nat Rev Mol Cell Biol 4, 915-25.
Burgess, T. et al (2006) Fully human monoclonal antibodies to hepatocyte growth factor with therapeutic potential against hepatocyte growth factor/c-Met-dependent human tumors. Cancer Res 66, 1721-9.
Di Renzo, M. F. et al (2000) Somatic mutations of the MET oncogene are selected during metastatic spread of human HNSC carcinomas. Oncogene 19, 1547-55.
Dyson M R, et al (2011) Mapping Protein Interactions by Combining Antibody Affinity Maturation and Mass Spectrometry Analytical Biochemistry 417 25-35.
Edwards B M, et al (2000) The remarkable flexibility of the human antibody repertoire; isolation of over one thousand different antibodies to a single protein. BLys Journal of Molecular Biology; 334:103-18.
Gherardi, E. et al (2003) Functional map and domain structure of MET, the product of the c-met protooncogene and receptor for hepatocyte growth factor/scatter factor. Proc Natl Acad Sci USA 100, 12039-44.
Gherardi, E. et al. (2006) Structural basis of hepatocyte growth factor/scatter factor and MET signalling. Proc Natl Acad Sci USA 103, 4046-51.
Graveel, C. et al (2004) Activating Met mutations produce unique tumour profiles in mice with selective duplication of the mutant allele. Proc Natl Acad Sci USA 101, 17198-203.
Holmes et al (2007). J. Mol. Biol. 367(2): 395-408.
Jeffers, M. et al (1996) Hepatocyte growth factor/scatter factor-Met signaling induces proliferation, migration, and morphogenesis of pancreatic oval cells. Cell Growth Differ 7, 1805-13.
Kong-Beltran, M. et al (2006) Somatic mutations lead to an oncogenic deletion of met in lung cancer. Cancer Res 66, 283-9.
Lorenzato, A. et al (2002) Novel somatic mutations of the MET oncogene in human carcinoma metastases activating cell motility and invasion. Cancer Res 62, 7025-30.
Martin C D, et al (2006) A simple vector system to improve performance and utilisation of recombinant antibodies. BMC Biotechnol., 6:46.
Niemann, H. H. et al (2007) Structure of the Human Receptor Tyrosine Kinase Met in Complex with the Listeria Invasion Protein InIB. Cell 130, 235-246.
Pershad K, et al (2010) Generating a panel of highly specific antibodies to 20 human SH2 domains by phage display. Protein Engineering, Design and Selection 23 (4) 279-288
Schmidt, L. et al (1997) Germline and somatic mutations in the tyrosine kinase domain of the MET proto-oncogene in papillary renal carcinomas. Nat Genet 16, 68-73.
Schofield D J, et al (2007) Application of phage display to high throughput antibody generation and characterization. Genome Biol. 8 (11).
Schwall, R. & Tabor, K. H. (U.S. Pat. No. 6,468,529).
Smolen et al, (2006) “Amplification of MET may identify a subset of cancers with extreme sensitivity to the selective tyrosine kinase inhibitor PHA-665752.” Proc Natl Acad Sci USA. 103(7): 2316-21.
Stamos, J., et al (2004) Crystal structure of the HGF beta-chain in complex with the Sema domain of the Met receptor. Embo J 23, 2325-35.
Studier FW: (2005) Protein production by auto-induction in high density shaking cultures. Protein Expr Purif 2005, 41(1):207-234.
Takayama, H. et al (1997) Diverse tumorigenesis associated with aberrant development in mice overexpressing hepatocyte growth factor/scatter factor. Proc Natl Acad Sci USA 94, 701-6.
Tom, Bisson, & Durocher (2007) “Transient Expression in HEK293-EBNA1 cells”, Chapter 12, Expression Systems, ed. Dyson, M. R & Durocher, Y. pub. Scion.
Wuhlfard S. et al (2010) Journal of Biotechnology 148: 128-132

1. An antibody that specifically binds to the extracellular domain of the human HGF receptor (MET) (SEQ ID No: 1), wherein the antibody comprises:
a heavy chain CDR1 comprising the amino acid sequence DYYMH (SEQ ID NO:2), or a variant thereof comprising 1, 2 or 3 amino acid substitutions,
a heavy chain CDR2 comprising the amino acid sequence LVDPEDGETIYAEKFQ (SEQ ID NO:3), or a variant thereof comprising 1, 2 or 3 amino acid substitutions,
a heavy chain CDR3 comprising the amino acid sequence DATTPYYGMDV (SEQ ID NO:4), or a variant thereof wherein
the Y at position 7 is replaced with W,
the G at position 8 is replaced with F,
the M at position 9 is replaced with P,
the D at position 10 is replaced with M, W, V, Q, R or Y, and/or
the V at position 11 is replaced with E, W, Q, S, T, L, E or L, or a combination thereof,
a light chain CDR1 comprising the amino acid sequence QASQDISNYLN (SEQ ID NO:13), or a variant thereof comprising 1, 2 or 3 amino acid substitutions,
a light chain CDR2 comprising the amino acid sequence DASNLET (SEQ ID NO:14), or a variant thereof comprising 1, 2 or 3 amino acid substitutions, and
a light chain CDR3 comprising the amino acid sequence QQGDSFPLT (SEQ ID NO:15), or a variant thereof comprising 1, 2 or 3 amino acid substitutions.

2-11. (canceled)

12. The antibody according to claim 1, wherein the amino acid substitutions are conservative amino acid substitutions.

13. The antibody according to claim 1, wherein the antibody comprises the amino acid sequence of SEQ ID NO: 26, or wherein the antibody comprises a heavy chain amino acid sequence of SEQ ID NO: 28 and a light chain amino acid sequence of SEQ ID NO: 29, or wherein the antibody comprises a heavy chain amino acid sequence of SEQ ID NO: 30 and a light chain amino acid sequence of SEQ ID NO: 31, or wherein the antibody comprises a heavy chain amino acid sequence of SEQ ID NO: 32 and a light chain amino acid sequence of SEQ ID NO: 31.

14. (canceled)

15. An The antibody according to claim 1, wherein the antibody specifically binds to amino acid residues 568-741 of human MET (SEQ ID No: 1).

16. The antibody according to claim 1, wherein the antibody binds to the extracellular of the human MET with an affinity of at least 1 nM, and preferably with an affinity of at least 0.5 nM.

17-22. (canceled)

23. An antibody that specifically binds to an epitope located within amino acid residues 592-615 of human MET (SEQ ID No: 1).

24-34. (canceled)

35. The antibody according to claim 1, wherein the antibody is an antigen-binding fragment of an antibody; or a domain antibody.

36. The antibody according to claim 35 wherein the antigen-binding fragment is an Fv fragment.

37. The antibody according to claim 35 wherein the antigen-binding fragment is a Fab-like fragment.

38. The antibody according to claim 1, wherein the antibody is a monovalent antibody.

39. The antibody according to claim 1, wherein the antibody is a monoclonal antibody.

40. The antibody according to claim 1, wherein the antibody is human or humanised.

41. The antibody according to claim 1, wherein the antibody is a recombinant antibody.

42. The antibody according to claim 1, wherein the antibody is in an isolated and/or purified form.

43. The antibody according to claim 1, wherein the antibody has been PEGylated.

44. A nucleic acid molecule encoding the antibody of claim 1.

45. A vector comprising the nucleic acid of claim 44.

46. A host cell comprising the vector of claim 45.

47. A compound comprising an antibody according to claim 1 and a detectable moiety.

48. The compound according to claim 47 wherein the detectable moiety comprises an enzyme, a radioactive atom, a fluorescent moiety, a chemiluminescent moiety or a bioluminescent moiety.

49. The compound according to claim 47 wherein the antibody is an scFv fragment.

50. A compound comprising an antibody according to claim 1 and a cytotoxic moiety.

51-52. (canceled)

53. The compound according to claim 50 wherein the antibody is an Fab fragment.

54. A pharmaceutical composition comprising the antibody according to claim 1, and a pharmaceutically acceptable diluent, carrier or excipient.

55. The pharmaceutical composition according to claim 1, further comprising at least one additional anti-cancer agent.

56. A kit of parts comprising the antibody according to claim 1 and at least one additional anti-cancer agent.

57-58. (canceled)

59. A method of inhibiting activity of human MET on a cell, of inhibiting human MET-mediated cellular proliferation, or of inhibiting human MET-mediated cellular migration, the method comprising contacting the cell with an effective amount of the antibody according to claim 1.

60. The method of claim 59 wherein the cell is a tumour cell.

61. (canceled)

62. The method of claim 59 which is performed on cells in vivo.

63. A method of inhibiting growth and/or metastasis of a tumour in a human patient, the method comprising administering to the patient a therapeutically effective amount of the antibody according to claim 1.

64. A method of treating cancer in a human patient, the method comprising administering to the patient a therapeutically effective amount of the antibody according to claim 1.

65-70. (canceled)

71. The method claim 60 wherein the tumour or cancer is breast cancer, pancreatic cancer, colon cancer, gastric cancer or lung cancer.

72. (canceled)

73. A method of detecting the presence of human MET on a cell, the method comprising contacting the cell with a compound according to claim 47 and detecting the detectable moiety.

74. The method of claim 73 wherein the cell is a tumour or cancer cell.

75. (canceled)

76. The method of claim 73 which is performed on cells in vivo.

77-78. (canceled)

79. The method according to claim 74, wherein the tumour or cancer is selected from bladder cancer, brain cancer, breast cancer, cervical cancer, colon cancer, colorectal cancer, endometrial cancer, gastric cancer, head and neck cancer, kidney cancer, liver cancer, lung cancer including small cell and non-small-cell lung cancer, medulloblastoma, nasopharyngeal cancer, gastric cancer, oesophageal cancer, osteosarcoma, ovarian cancer, pancreatic cancer, papillary renal cell carcinoma, prostate cancer, rhabdomyosarcoma, stomach cancer, thyroid cancer, cholangiocarcinoma, osteosarcoma, rhabdomyosarcoma, synovial sarcoma, Kaposi's sarcoma, leiomyosarcomas, fibrosarcoma, leukaemia including acute myelogenous leukaemia, adult T-cell leukaemia and chronic myeloid leukaemia, lymphoma, multiple myeloma, melanoma, mesothelioma, Wilms tumour, glioblastomata and astrocytoma.

80-90. (canceled)

91. A method of treating endometriosis, rheumatoid arthritis, or hypoglycaemia in a human patient, or for treating, reversing or minimising side-effects associated with the use of a Met-agonist in a human patient, the method comprising administering to the patient a therapeutically effective amount of the antibody according to claim 1.

92-94. (canceled)

95. The antibody according to claim 36 wherein the Fv fragment is a single chain Fv or a disulphide-bonded Fv.

96. The antibody according to claim 37 wherein the Fab Fab-like fragment is an Fab fragment, an Fab′ fragment, or an F(ab)₂ fragment.


SEQUENCE LISTING (201 sequences)

SEQ ID NO: 1 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 2 - protein, 5 aa, Artificial Sequence
  Heavy chain CDR1
DYYMH

SEQ ID NO: 3 - protein, 16 aa, Artificial Sequence
  Heavy chain CDR2
LVDPEDGETIYAEKFQ

SEQ ID NO: 4 - protein, 11 aa, Artificial Sequence
  Heavy chain CDR3
DATTPYYGMDV

SEQ ID NO: 5 - protein, 11 aa, Artificial Sequence
  Heavy chain CDR3 (107 A8)
DATTPYWGMVE

SEQ ID NO: 6 - protein, 11 aa, Artificial Sequence
  Heavy chain CDR3 (107 A7)
DATTPYWGMMW

SEQ ID NO: 7 - protein, 11 aa, Artificial Sequence
  Heavy chain CDR3 (111 D6)
DATTPYWGMWQ

SEQ ID NO: 8 - protein, 11 aa, Artificial Sequence
  Heavy chain CDR3 (107 A1)
DATTPYWGMVS

SEQ ID NO: 9 - protein, 11 aa, Artificial Sequence
  Heavy chain CDR3 (110 A10)
DATTPYWGMQT

SEQ ID NO: 10 - protein, 11 aa, Artificial Sequence
  Heavy chain CDR3 (110 A6)
DATTPYWGMQL

SEQ ID NO: 11 - protein, 11 aa, Artificial Sequence
  Heavy chain CDR3 (110 A12)
DATTPYWFPRE

SEQ ID NO: 12 - protein, 11 aa, Artificial Sequence
  Heavy chain CDR3 (110 A1)
DATTPYWFPYL

SEQ ID NO: 13 - protein, 11 aa, Artificial Sequence
  Light chain CDR1
QASQDISNYLN

SEQ ID NO: 14 - protein, 7 aa, Artificial Sequence
  Light chain CDR2
DASNLET

SEQ ID NO: 15 - protein, 9 aa, Artificial Sequence
  Light chain CDR3
QQGDSFPLT

SEQ ID NO: 16 - protein, 120 aa, Artificial Sequence
  Heavy chain variable region
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYYGMDVWGQGTLVTVSS

SEQ ID NO: 17 - protein, 120 aa, Artificial Sequence
  Heavy chain variable region
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYWGMVEWGQGTLVTVSS

SEQ ID NO: 18 - protein, 120 aa, Artificial Sequence
  Heavy chain variable region
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYWGMMWWGQGTLVTVSS

SEQ ID NO: 19 - protein, 120 aa, Artificial Sequence
  Heavy chain variable region
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYWGMWQWGQGTLVTVSS

SEQ ID NO: 20 - protein, 120 aa, Artificial Sequence
  Heavy chain variable region
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYWGMVSWGQGTLVTVSS

SEQ ID NO: 21 - protein, 120 aa, Artificial Sequence
  Heavy chain variable region
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYWGMQTWGQGTLVTVSS

SEQ ID NO: 22 - protein, 120 aa, Artificial Sequence
  Heavy chain variable region
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYWGMQLWGQGTLVTVSS

SEQ ID NO: 23 - protein, 120 aa, Artificial Sequence
  Heavy chain variable region
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYWFPREWGQGTLVTVSS

SEQ ID NO: 24 - protein, 120 aa, Artificial Sequence
  Heavy chain variable region
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYWFPYLWGQGTLVTVSS

SEQ ID NO: 25 - protein, 107 aa, Artificial Sequence
  Light chain variable region
DIQMTQSPSSLSASVGDRVTITCQASQDISNYLNWYQQKPGRAPKVLIYDASNLETGVPSRFSGSGSGTEFTLTISNLRPDDFATYYCQQGDSFPLTFGGGTKVEIK

SEQ ID NO: 26 - protein, 244 aa, Artificial Sequence
  Antibody 7A2 scFv
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYYGMDVWGQGTLVTVSSLEGGGGSGGGGSGGGASDIQMTQSPSSLSASVGDRVTITCQASQDISNYLNWYQQKPGRAPKVLIYDASNLETGVPSRFSGSGSGTEFTLTISNLRPDDFATYYCQQGDSFPLTFGGGTKVEIK

SEQ ID NO: 27 - protein, 187 aa, Homo sapiens
EAEAYVEGQRKRRNTIHEFKKSAKTTLIKIDPALKIKTKKVNTADQCADRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVE

SEQ ID NO: 28 - protein, 231 aa, Artificial Sequence
  7A2 Fab heavy chain amino acid sequence
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYYGMDVWGQGTLVTVSSLEASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCLQGGGS

SEQ ID NO: 29 - protein, 225 aa, Artificial Sequence
  7A2 Fab light chain amino acid sequence
ASDIQMTQSPSSLSASVGDRVTITCQASQDISNYLNWYQQKPGRAPKVLIYDASNLETGVPSRFSGSGSGTEFTLTISNLRPDDFATYYCQQGDSFPLTFGGGTKVEIKRAAATVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECSRASGS

SEQ ID NO: 30 - protein, 223 aa, Artificial Sequence
  107-A07 Fab heavy chain amino acid sequence
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYWGMMWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 31 - protein, 214 aa, Artificial Sequence
  107-A07 Fab light chain amino acid sequence
DIQMTQSPSSLSASVGDRVTITCQASQDISNYLNWYQQKPGRAPKVLIYDASNLETGVPSRFSGSGSGTEFTLTISNLRPDDFATYYCQQGDSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 32 - protein, 446 aa, Artificial Sequence
  107_A07 IgG2 heavy chain
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYWGMMWWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 220 aa, Homo sapiens
CRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYR

SEQ ID NO: 34 - protein, 60 aa, Homo sapiens
PNSAPLEGGTRLTICGWDFGFRRNNKFDLKKTRVLLGNESCTLTLSESTMNTLKCTVGPA

SEQ ID NO: 35 - protein, 60 aa, Mus Musculus
PTSAPLEGGTVLTICGWDFGFRKNNKFDLRKTKVLLGNESCTLTLSESTTNTLKCTVGPA

SEQ ID NO: 36 - protein, 11 aa, Artificial Sequence
  107_A10
DATTPYWGMQE

SEQ ID NO: 37 - protein, 11 aa, Artificial Sequence
  107_A11
DATTPYWGMAW

SEQ ID NO: 38 - protein, 11 aa, Artificial Sequence
  107_A12
DATTPYWWMAK

SEQ ID NO: 39 - protein, 11 aa, Artificial Sequence
  107_A2
DATTPYWGMQS

SEQ ID NO: 40 - protein, 11 aa, Artificial Sequence
  107_A3
DATTAYWGMQK

SEQ ID NO: 41 - protein, 11 aa, Artificial Sequence
  107_A4
DATTPYWFPYH

SEQ ID NO: 42 - protein, 11 aa, Artificial Sequence
  107_A5
DATTPYWGMSK

SEQ ID NO: 43 - protein, 11 aa, Artificial Sequence
  107_A6
DATTPYWTPFV

SEQ ID NO: 44 - protein, 11 aa, Artificial Sequence
  107_A9
DATTPYWGMMT

SEQ ID NO: 45 - protein, 11 aa, Artificial Sequence
  107_B1
DATHIYWNMDI

SEQ ID NO: 46 - protein, 11 aa, Artificial Sequence
  107_B10
DATTPYWGMRV

SEQ ID NO: 47 - protein, 11 aa, Artificial Sequence
  107_B11
DATTPYWGMAM

SEQ ID NO: 48 - protein, 11 aa, Artificial Sequence
  107_B12
DATTPYWGMTA

SEQ ID NO: 49 - protein, 11 aa, Artificial Sequence
  107_B2
DATTPYWGMQM

SEQ ID NO: 50 - protein, 11 aa, Artificial Sequence
  107_B3
DATTPYWGMKM

SEQ ID NO: 51 - protein, 11 aa, Artificial Sequence
  107_B4
DATTPYWFPNV

SEQ ID NO: 52 - protein, 11 aa, Artificial Sequence
  107_B5
DATTPYWGMQI

SEQ ID NO: 53 - protein, 11 aa, Artificial Sequence
  107_B6
DATTPYWGMQK

SEQ ID NO: 54 - protein, 11 aa, Artificial Sequence
  107_B7
DATTPYWCMRA

SEQ ID NO: 55 - protein, 11 aa, Artificial Sequence
  107_B8
DATTPYWGMAE

SEQ ID NO: 56 - protein, 11 aa, Artificial Sequence
  107_C1
DATTPYWGMSM

SEQ ID NO: 57 - protein, 11 aa, Artificial Sequence
  107_C2
DATTPYWGMVK

SEQ ID NO: 58 - protein, 11 aa, Artificial Sequence
  107_C4
DATTPYWSPWM

SEQ ID NO: 59 - protein, 11 aa, Artificial Sequence
  107_C5
DATTPYWYPIV

SEQ ID NO: 60 - protein, 11 aa, Artificial Sequence
  107_C6
DATTPYWGMSV

SEQ ID NO: 61 - protein, 11 aa, Artificial Sequence
  108_C11
DATTPYWFPII

SEQ ID NO: 62 - protein, 11 aa, Artificial Sequence
  108_C12
DATTPYWGMAS

SEQ ID NO: 63 - protein, 11 aa, Artificial Sequence
  108_C8
DATTPYWFPYI

SEQ ID NO: 64 - protein, 11 aa, Artificial Sequence
  108_C9
DATTPYWFPRB

SEQ ID NO: 65 - protein, 11 aa, Artificial Sequence
  108_D1
DATTPYWTPWK

SEQ ID NO: 66 - protein, 11 aa, Artificial Sequence
  108_D10
DATTPYWGMSH

SEQ ID NO: 67 - protein, 11 aa, Artificial Sequence
  108_D11
DATTPYWGAAA

SEQ ID NO: 68 - protein, 11 aa, Artificial Sequence
  108_D12
DATTPYWTPYV

SEQ ID NO: 69 - protein, 11 aa, Artificial Sequence
  108_D2
DATTPYWAPWV

SEQ ID NO: 70 - protein, 11 aa, Artificial Sequence
  108_D3
DATTPYWFPYV

SEQ ID NO: 71 - protein, 11 aa, Artificial Sequence
  108_D4
DATTPYWGMSG

SEQ ID NO: 72 - protein, 11 aa, Artificial Sequence
  108_D5
DATTPYWSPHT

SEQ ID NO: 73 - protein, 11 aa, Artificial Sequence
  108_D7
DATTPYWGMDM

SEQ ID NO: 74 - protein, 11 aa, Artificial Sequence
  108_D9
DATTPYWYPYV

SEQ ID NO: 75 - protein, 11 aa, Artificial Sequence
  108_E1
DATTPYWFPLV

SEQ ID NO: 76 - protein, 11 aa, Artificial Sequence
  108_E10
DATTPYWFPWV

SEQ ID NO: 77 - protein, 11 aa, Artificial Sequence
  108_E11
DATTPYCSRTS

SEQ ID NO: 78 - protein, 11 aa, Artificial Sequence
  108_E12
DATTPYWLAAE

SEQ ID NO: 79 - protein, 11 aa, Artificial Sequence
  108_E2
DATTPYWTPWV

SEQ ID NO: 80 - protein, 11 aa, Artificial Sequence
  108_E4
DATTPYWGMIW

SEQ ID NO: 81 - protein, 11 aa, Artificial Sequence
  108_E5
DATTPYWGMWI

SEQ ID NO: 82 - protein, 11 aa, Artificial Sequence
  108_E7
DATTPYWGMWR

SEQ ID NO: 83 - protein, 11 aa, Artificial Sequence
  108_E8
DATTPYWWMQK

SEQ ID NO: 84 - protein, 11 aa, Artificial Sequence
  108_F1
DATTPYWVPRF

SEQ ID NO: 85 - protein, 11 aa, Artificial Sequence
  108_F11
DGSNLSWQMDV

SEQ ID NO: 86 - protein, 11 aa, Artificial Sequence
  108_F2
DATTPYWGMSS

SEQ ID NO: 87 - protein, 11 aa, Artificial Sequence
  108_F3
DATTPYWGMAK

SEQ ID NO: 88 - protein, 11 aa, Artificial Sequence
  108_F6
DATTPYCCTIE

SEQ ID NO: 89 - protein, 11 aa, Artificial Sequence
  108_G1
DATTPYWFPRM

SEQ ID NO: 90 - protein, 11 aa, Artificial Sequence
  108_G3
DATTPYWAPWR

SEQ ID NO: 91 - protein, 11 aa, Artificial Sequence
  108_G4
DATTPYWFPRF

SEQ ID NO: 92 - protein, 11 aa, Artificial Sequence
  108_G5
DATTPYWWMQS

SEQ ID NO: 93 - protein, 11 aa, Artificial Sequence
  108_G6
DATTPYWFPVW

SEQ ID NO: 94 - protein, 11 aa, Artificial Sequence
  109_G10
DATTPYWFPRV

SEQ ID NO: 95 - protein, 11 aa, Artificial Sequence
  109_G11
DATTPYWWTAN

SEQ ID NO: 96 - protein, 11 aa, Artificial Sequence
  109_G12
DATTPYWGMAA

SEQ ID NO: 97 - protein, 11 aa, Artificial Sequence
  109_G8
DATTPYWGMAT

SEQ ID NO: 98 - protein, 11 aa, Artificial Sequence
  109_G9
DATTPYWGMQE

SEQ ID NO: 99 - protein, 11 aa, Artificial Sequence
  109_H1
DATTPYWGVQK

SEQ ID NO: 100 - protein, 11 aa, Artificial Sequence
  109_H11
DATTPYWGMAQ

SEQ ID NO: 101 - protein, 11 aa, Artificial Sequence
  109_H12
DATTPYYGMDV

SEQ ID NO: 102 - protein, 11 aa, Artificial Sequence
  109_H2
DATTPYWGMWE

SEQ ID NO: 103 - protein, 11 aa, Artificial Sequence
  109_H3
DATTPYWGMQQ

SEQ ID NO: 104 - protein, 11 aa, Artificial Sequence
  109_H5
DATTPYWGAQE

SEQ ID NO: 105 - protein, 11 aa, Artificial Sequence
  109_H6
DATTPYWFPRT

SEQ ID NO: 106 - protein, 11 aa, Artificial Sequence
  109_H7
DATTPYWFPRW

SEQ ID NO: 107 - protein, 11 aa, Artificial Sequence
  109_H8
DATTPYWFPRS

SEQ ID NO: 108 - protein, 11 aa, Artificial Sequence
  109_H9
DATTPYWGMTK

SEQ ID NO: 109 - protein, 11 aa, Artificial Sequence
  110_A2
DATTPYWGMST

SEQ ID NO: 110 - protein, 11 aa, Artificial Sequence
  110_A3
DATTPYWGVQV

SEQ ID NO: 111 - protein, 11 aa, Artificial Sequence
  110_A5
DATTPYWYPFV

SEQ ID NO: 112 - protein, 11 aa, Artificial Sequence
  110_A8
DATTPYWCMAI

SEQ ID NO: 113 - protein, 11 aa, Artificial Sequence
  110_A9
DATTPYWGMAG

SEQ ID NO: 114 - protein, 11 aa, Artificial Sequence
  110_B1
DATTPYWGMBL

SEQ ID NO: 115 - protein, 11 aa, Artificial Sequence
  110_B10
DATTPYWGMBM

SEQ ID NO: 116 - protein, 11 aa, Artificial Sequence
  110_B3
DMELNYYGMDV

SEQ ID NO: 117 - protein, 11 aa, Artificial Sequence
  110_B4
DATTPYWFPBT

SEQ ID NO: 118 - protein, 11 aa, Artificial Sequence
  110_B5
DATTPYWGMMS

SEQ ID NO: 119 - protein, 11 aa, Artificial Sequence
  110_B6
DTDLKYYGMDV

SEQ ID NO: 120 - protein, 11 aa, Artificial Sequence
  110_B7
DGLWMYYGMDV

SEQ ID NO: 121 - protein, 11 aa, Artificial Sequence
  110_B8
DATTPYWGMML

SEQ ID NO: 122 - protein, 11 aa, Artificial Sequence
  110_B9
DATTPYWSPIW

SEQ ID NO: 123 - protein, 11 aa, Artificial Sequence
  111_B12
DATTPYWGAQA

SEQ ID NO: 124 - protein, 11 aa, Artificial Sequence
  111_C1
DATTPYWSPWA

SEQ ID NO: 125 - protein, 11 aa, Artificial Sequence
  111_C10
DATTPYWTPEI

SEQ ID NO: 126 - protein, 11 aa, Artificial Sequence
  111_C12
DATTPYWFPRY

SEQ ID NO: 127 - protein, 11 aa, Artificial Sequence
  111_C3
DATTPYWGMQG

SEQ ID NO: 128 - protein, 11 aa, Artificial Sequence
  111_C5
DATTPYWFPKV

SEQ ID NO: 129 - protein, 11 aa, Artificial Sequence
  111_C6
DATTPYWFPQH

SEQ ID NO: 130 - protein, 11 aa, Artificial Sequence
  111_C8
DATTPYWFPVV

SEQ ID NO: 131 - protein, 11 aa, Artificial Sequence
  111_C9
DATTPYWGMMA

SEQ ID NO: 132 - protein, 11 aa, Artificial Sequence
  111_D1
DATTPYWGMDE

SEQ ID NO: 133 - protein, 11 aa, Artificial Sequence
  111_D11
DATTPYWGMMR

SEQ ID NO: 134 - protein, 11 aa, Artificial Sequence
  111_D2
DATTPYWFPWI

SEQ ID NO: 135 - protein, 11 aa, Artificial Sequence
  111_D3
DATTPYWGAAL

SEQ ID NO: 136 - protein, 11 aa, Artificial Sequence
  111_D4
DATTPYWGMWV

SEQ ID NO: 137 - protein, 11 aa, Artificial Sequence
  111_D5
DATTPYWFPAV

SEQ ID NO: 138 - protein, 11 aa, Artificial Sequence
  111_D7
DATTPYWFPYA

SEQ ID NO: 139 - protein, 11 aa, Artificial Sequence
  111_D8
DATTPYWGMSW

SEQ ID NO: 140 - protein, 11 aa, Artificial Sequence
  111_E10
DATTPYWYPYA

SEQ ID NO: 141 - protein, 11 aa, Artificial Sequence
  111_E11
DATTPYWGMRE

SEQ ID NO: 142 - protein, 11 aa, Artificial Sequence
  111_E12
DATTPYWSPWV

SEQ ID NO: 143 - protein, 11 aa, Artificial Sequence
  111_E3
DATTPYWFPVE

SEQ ID NO: 144 - protein, 11 aa, Artificial Sequence
  111_E4
DATTPYWGIEV

SEQ ID NO: 145 - protein, 11 aa, Artificial Sequence
  111_E5
DATTPYWGMCE

SEQ ID NO: 146 - protein, 11 aa, Artificial Sequence
  111_E6
DATTPYWGMAV

SEQ ID NO: 147 - protein, 11 aa, Artificial Sequence
  111_E7
DATTPYWGMMM

SEQ ID NO: 148 - protein, 11 aa, Artificial Sequence
  111_E8
DATTPYWFPYR

SEQ ID NO: 149 - protein, 11 aa, Artificial Sequence
  112_F1
DATTPYWGMAR

SEQ ID NO: 150 - protein, 11 aa, Artificial Sequence
  112_F11
DATTPYWGMMQ

SEQ ID NO: 151 - protein, 11 aa, Artificial Sequence
  112_F12
DATTPYWGMQW

SEQ ID NO: 152 - protein, 11 aa, Artificial Sequence
  112_F4
DATTPYWGMAD

SEQ ID NO: 153 - protein, 11 aa, Artificial Sequence
  112_F8
DATTPYWTPFL

SEQ ID NO: 154 - protein, 10 aa, Artificial Sequence
  112_F9
DATTPYWTWV

SEQ ID NO: 155 - protein, 11 aa, Artificial Sequence
  112_G1
DATTPYWGMWH

SEQ ID NO: 156 - protein, 11 aa, Artificial Sequence
  112_G11
DATTPYWFPYT

SEQ ID NO: 157 - protein, 11 aa, Artificial Sequence
  112_G3
DATTPYWGMSA

SEQ ID NO: 158 - protein, 11 aa, Artificial Sequence
  112_G4
DATTPYWGAQS

SEQ ID NO: 159 - protein, 11 aa, Artificial Sequence
  112_G5
DATTPYWGMRS

SEQ ID NO: 160 - protein, 11 aa, Artificial Sequence
  112_G6
DATTPYWFPFV

SEQ ID NO: 161 - protein, 11 aa, Artificial Sequence
  112_G7
DATTPYWFPQT

SEQ ID NO: 162 - protein, 11 aa, Artificial Sequence
  112_G9
DATTPYWGMQR

SEQ ID NO: 163 - protein, 11 aa, Artificial Sequence
  112_H1
DATTPYWGAAV

SEQ ID NO: 164 - protein, 11 aa, Artificial Sequence
  112_H10
DATTPYWGIRK

SEQ ID NO: 165 - protein, 11 aa, Artificial Sequence
  112_H11
DATTPYWFPQV

SEQ ID NO: 166 - protein, 11 aa, Artificial Sequence
  112_H12
DATTPYWFPYK

SEQ ID NO: 167 - protein, 11 aa, Artificial Sequence
  112_H2
DATTPYWGMQV

SEQ ID NO: 168 - protein, 11 aa, Artificial Sequence
  112_H4
DATTPYWGMWK

SEQ ID NO: 169 - protein, 11 aa, Artificial Sequence
  112_H5
DATTPYWGMQA

SEQ ID NO: 170 - protein, 11 aa, Artificial Sequence
  112_H6
DATTPYWGMME

SEQ ID NO: 171 - protein, 11 aa, Artificial Sequence
  112_H7
DATTPYWGVQS

SEQ ID NO: 172 - protein, 11 aa, Artificial Sequence
  112_H8
DATTPYWLPLY

SEQ ID NO: 173 - protein, 11 aa, Artificial Sequence
  112_H9
DATTPYWFPYS

SEQ ID NO: 174 - protein, 20 aa, Artificial Sequence
  P2A sequence
GSGATNFSLLKQAGDVEENP

SEQ ID NO: 175 - protein, 259 aa, Artificial Sequence
  7A2 sequence tagged
EAEAQMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYYGMDVWGQGTLVTVSSLEGGGGSGGGGSGGGASDIQMTQSPSSLSASVGDRVTITCQASQDISNYLNWYQQKPGRAPKVLIYDASNLETGVPSRFSGSGSGTEFTLTISNLRPDDFATYYCQQGDSFPLTFGGGTKVEIKRPRHHHHHHHH

SEQ ID NO: 176 - protein, 219 aa, Artificial Sequence
  D1.3 Fab heavy chain amino acid sequence
QVQLQESGPGLVRPSQTLSLTCTVSGSTFSGYGVNWVRQPPGRGLEWIGMIWGDGNTDYNSALKSRVTMLVDTSKNQFSLRLSSVTAADTAVYYCARERDYRLDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 177 - protein, 214 aa, Artificial Sequence
  D1.3 Fab light chain amino acid sequence
DIQMTQSPSSLSASVGDRVTITCRASGNIHNYLAWYQQKPGKAPKLLIYYTTTLADGVPSRFSGSGSGTDFTFTISSLQPEDIATYYCQHFWSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 178 - DNA, 735 bp, Artificial Sequence
  DNA sequence of anti-Met antibody clone 7A2
caaatgcagctggtacagtctggggctgaggtgaagaagcctggggccccagtgaaggtctcctgcaaggtttctggatacaccttcaccgactactacatgcactgggtgcaacaggcccctggaaaagggcttgagtggatgggacttgttgatcctgaagatggtgaaacaatatacgcagagaagttccagggcagagtcaccataaccgcggacacgtctacagacacagcctacatggagctgagcagcctgagatctgaggacacggccgtgtattactgtgcaacagatgctacgacgccctactacggtatggacgtctggggccaaggaaccctggtcaccgtctcctcactcgagggtggaggcggttcaggcggaggtggctctggcggtggcgctagcgacatccagatgacccagtctccatcctccctgtctgcatctgtaggagacagagtcaccatcacttgccaggcgagtcaggacattagcaactatttaaattggtatcagcagaaaccagggagagcccctaaggtcctgatctacgatgcatccaatttggaaacaggggtcccatcaaggttcagcggcagtggatctgggacagagttcactctcaccatcagcaacctccggcctgatgattttgcaacttactattgtcaacaaggtgacagtttcccgctcactttcggcggagggaccaaggtggaaatcaaacgt

SEQ ID NO: 179 - protein, 245 aa, Artificial Sequence
  Amino acid sequence of anti-Met antibody clone 7A2
QMQLVQSGAEVKKPGAPVKVSCKVSGYTFTDYYMHWVQQAPGKGLEWMGLVDPEDGETIYAEKFQGRVTITADTSTDTAYMELSSLRSEDTAVYYCATDATTPYYGMDVWGQGTLVTVSSLEGGGGSGGGGSGGGASDIQMTQSPSSLSASVGDRVTITCQASQDISNYLNWYQQKPGRAPKVLIYDASNLETGVPSRFSGSGSGTEFTLTISNLRPDDFATYYCQQGDSFPLTFGGGTKVEIKR

SEQ ID NO: 180 - protein, 54 aa, Artificial Sequence
  7A2 CDR3 heavy region
RVTITADTSTDTAYMELSSLRSEDTAVYYCATDATTPYYGMDVWGQGTLVTVSS

SEQ ID NO: 181 - protein, 51 aa, Artificial Sequence
  7A2 CDR3 light region
GVPSRFSGSGSGTEFTLTISNLRPDDFATYYCQQGDSFPLTFGGGTKVEIK

SEQ ID NO: 182 - DNA, 63 bp, Artificial Sequence
  Oligos for VH CDR3 Mutagenesis (VH3.1)
acggccgtgtattactgtgcaacannsnnsnnsnnsnnstactacggtatggacgtctggggc

SEQ ID NO: 183 - DNA, 72 bp, Artificial Sequence
  Oligos for VH CDR3 Mutagenesis (VH3.2)
acggccgtgtattactgtgcaacagatgctacgnnsnnsnnsnnsnnsatggacgtctggggccaaggaacc

SEQ ID NO: 184 - DNA, 62 bp, Artificial Sequence
  Oligos for VH CDR3 Mutagenesis (VH3.3)
gcaacagatgctacgacgccctacnnsnnsnnsnnsnnstggggccaaggaaccctggtcac

SEQ ID NO: 185 - DNA, 24 bp, Artificial Sequence
  Reconstruction primer for VH 3.1 and VH 3.2
tgttgcacagtaatacacggccgt

SEQ ID NO: 186 - DNA, 24 bp, Artificial Sequence
  Reconstruction primer for VH 3.3
gtagggcgtcgtagcatctgttgc

SEQ ID NO: 187 - DNA, 71 bp, Artificial Sequence
  Oligos for VL CDR3 Mutagenesis (VL3.1)
cggcctgatgattttgcaacttactattgtnnsnnsnnsnnsnnsnnsccgctcactttcggcggagggac

SEQ ID NO: 188 - DNA, 72 bp, Artificial Sequence
  Oligos for VL CDR3 Mutagenesis (VL3.2)
gattttgcaacttactattgtcaacaaggtnnsnnsnnsnnsnnsnnsttcggcggagggaccaaggtggaa

SEQ ID NO: 189 - DNA, 30 bp, Artificial Sequence
  Reconstruction primer for VL 3.1
acaatagtaagttgcaaaatcatcaggccg

SEQ ID NO: 190 - DNA, 27 bp, Artificial Sequence
  Reconstruction primer for VL 3.2
accttgttgacaatagtaagttgcaaa

SEQ ID NO: 191 - protein, 120 aa, Artificial Sequence; identical to SEQ ID NO: 16
  Met 7A2 (heavy chain sequence)

SEQ ID NO: 192 - protein, 120 aa, Artificial Sequence; identical to SEQ ID NO: 22
  110_A06 (heavy chain sequence)

SEQ ID NO: 193 - protein, 120 aa, Artificial Sequence; identical to SEQ ID NO: 21
  110_A10 (heavy chain sequence)

SEQ ID NO: 194 - protein, 120 aa, Artificial Sequence; identical to SEQ ID NO: 20
  107_A01 (heavy chain sequence)

SEQ ID NO: 195 - protein, 120 aa, Artificial Sequence; identical to SEQ ID NO: 17
  107_A08 (heavy chain sequence)

SEQ ID NO: 196 - protein, 120 aa, Artificial Sequence; identical to SEQ ID NO: 18
  107_A07 (heavy chain sequence)

SEQ ID NO: 197 - protein, 120 aa, Artificial Sequence; identical to SEQ ID NO: 19
  111_D06 (heavy chain sequence)

SEQ ID NO: 198 - protein, 120 aa, Artificial Sequence; identical to SEQ ID NO: 24
  110_A01 (heavy chain sequence)

SEQ ID NO: 199 - protein, 120 aa, Artificial Sequence; identical to SEQ ID NO: 23
  110_A12 (heavy chain sequence)

SEQ ID NO: 200 - protein, 17 aa, Artificial Sequence
  LINKER
LEGGGGSGGGGSGGGAS

SEQ ID NO: 201 - protein, 108 aa, Artificial Sequence
  Met 7A2 (light chain sequence)
DIQMTQSPSSLSASVGDRVTITCQASQDISNYLNWYQQKPGRAPKVLIYDASNLETGVPSRFSGSGSGTEFTLTISNLRPDDFATYYCQQGDSFPLTFGGGTKVEIKR